(19) United States
(12) Patent Application Publication
(10) Pub. No.: US 2026/0335392 A1
(43) Pub. Date: Oct. 29, 2026

(54) High-Polyphenol Olive Oil Nutraceutical Delivery Platform, Compositions, and Methods of Manufacture

(71) Applicant: Bedis Zormati, New York, NY (US)

(72) Inventor: Bedis Zormati, New York, NY (US)

(21) Appl. No.: 19/736,035

(22) Filed: Jul. 9, 2026

Related U.S. Application Data
(63) Continuation-in-part of application No. 19/576,353, filed on Mar. 24, 2026.

(51) Int. Cl. A61K 36/63; A61K 9/48; A61K 45/06

(52) U.S. Cl. CPC A61K 36/63; A61K 9/4808; A61K 45/06

(57) ABSTRACT

A nutraceutical delivery platform and associated systems, methods, and compositions for preserving and delivering high-polyphenol olive oil are disclosed. The platform includes selection of olive oil based on predetermined biochemical characteristics, analytical characterization of naturally occurring phenolic compounds, encapsulation of the selected olive oil within one or more protective layers, and incorporation of the encapsulated olive oil into orally administrable nutraceutical dosage forms together with one or more nutraceutical ingredients. The encapsulated olive oil functions as a biologically active lipid delivery matrix configured to preserve naturally occurring phenolic constituents during manufacturing, storage, packaging, and administration while providing controlled release of the olive oil and associated nutraceutical ingredients. The platform further includes manufacturing methodologies, quality characterization systems, preservation architectures, configurable dosage-form implementations, computer-implemented manufacturing management and quality-control systems, packaging and storage systems, and controlled-release delivery mechanisms. The disclosed technology is adaptable to multiple oral dosage forms including tablets, lozenges, gummies, capsules, powders, oral films, sachets, beverages, and related nutraceutical products while maintaining preservation of the naturally occurring biochemical profile of the high-polyphenol olive oil.

### CROSS REFERENCE TO RELATED APPLICATIONS

This application claims the benefit of priority under 35 U.S.C. § 119(e) to U.S. Provisional Patent Application No. 63/923,596, titled “Nutraceutical Lozenge Containing Microencapsulated Extra Virgin Olive Oil,” filed on Nov. 23, 2025, the entire contents of which are hereby incorporated by reference in their entirety for all purposes.

The present application is a Continuation-in-Part (CIP) of U.S. patent application Ser. No. 19/576,353, filed Mar. 24, 2026, entitled “Lipid-Based Nutraceutical Delivery Matrix Comprising Microencapsulated Olive Oil for Oral Dosage Forms,” the disclosure of which is incorporated herein by reference in its entirety.

The present Continuation-in-Part application includes subject matter disclosed in the parent application together with additional embodiments, systems, methods, manufacturing architectures, encapsulation technologies, preservation systems, delivery mechanisms, dosage-form implementations, computer-implemented management systems, formulation platforms, and other improvements not previously disclosed in the parent application. Accordingly, the present application claims the benefit of the filing date of the parent application only with respect to subject matter that is adequately supported therein, while newly added subject matter is entitled to the filing date of the present Continuation-in-Part application.

### FIELD OF THE INVENTION

The present invention relates generally to nutraceutical, pharmaceutical, and functional food delivery systems and, more particularly, to compositions, formulations, delivery platforms, and methods for stabilizing, preserving, and delivering high-polyphenol olive oil and associated bioactive compounds in oral dosage forms. More specifically, the invention pertains to lipid-based delivery platforms utilizing microencapsulated high-polyphenol olive oil as a multifunctional bioactive carrier matrix for nutraceutical ingredients including vitamins, minerals, botanical extracts, adaptogens, amino acids, peptides, probiotics, prebiotics, functional lipids, antioxidants, and other health-promoting compounds.

In particular embodiments, the invention relates to delivery platforms in which olive oil possessing elevated concentrations of naturally occurring phenolic compounds, including hydroxytyrosol, tyrosol, oleuropein, oleocanthal, oleacein, ligstroside derivatives, phenolic acids, flavonoids, tocopherols, phytosterols, and triterpenes, is microencapsulated to form a stable particulate lipid matrix that preserves the biological activity of the olive oil while facilitating incorporation into solid and semi-solid oral dosage forms. The invention further relates to technologies for maintaining phenolic integrity during harvesting, extraction, processing, encapsulation, formulation, storage, transportation, and administration, thereby improving oxidative stability, shelf life, sensory characteristics, and delivery performance.

The invention additionally relates to advanced encapsulation technologies including microencapsulation, nanoencapsulation, liposomal encapsulation, cyclodextrin inclusion complexes, nanoemulsions, microemulsions, coacervates, lipid nanoparticles, solid lipid particles, protein-based encapsulation systems, polysaccharide matrices, hydrocolloid systems, and combinations thereof for protecting high-polyphenol olive oil and enhancing the stability, dispersibility, controlled release, and bioavailability of olive oil-derived bioactive compounds and co-formulated nutraceutical ingredients.

The invention further relates to nutraceutical delivery platforms that are substantially independent of dosage form and are adaptable for incorporation into lozenges, fast-dissolving tablets, chewable tablets, oral films, gummies, capsules, powders, granules, sachets, nutritional beverages, functional confectionery products, softgels, and other ingestible compositions while maintaining the structural and functional characteristics of the microencapsulated high-polyphenol olive oil delivery matrix.

The present invention also relates to methods of manufacturing, preserving, formulating, and utilizing high-polyphenol olive oil delivery systems, including methods for selecting olive oils based on predetermined phenolic profiles, preserving naturally occurring phenolic compounds throughout manufacturing and storage, and producing consumer-friendly nutraceutical compositions capable of delivering both the intrinsic biological benefits of high-polyphenol olive oil and the complementary physiological benefits of additional nutraceutical active ingredients.

### BACKGROUND OF THE INVENTION

The global nutraceutical, dietary supplement, functional food, and wellness industries have experienced substantial growth as consumers increasingly seek natural products capable of promoting long-term health, disease prevention, healthy aging, and overall physiological well-being. Modern consumers are progressively shifting away from synthetic pharmaceutical interventions for routine health maintenance and are instead adopting nutraceutical compositions containing naturally derived bioactive compounds, botanical extracts, vitamins, minerals, probiotics, functional lipids, and antioxidants. This growing demand has accelerated research into naturally occurring compounds capable of providing multiple physiological benefits while maintaining high safety profiles and consumer acceptance.

Among naturally occurring functional lipids, olive oil has attracted significant scientific and commercial interest due to its unique biochemical composition and its longstanding association with the Mediterranean diet. Numerous scientific investigations have identified olive oil, particularly extra virgin olive oil, as an abundant source of biologically active compounds including monounsaturated fatty acids, polyphenols, hydroxytyrosol, tyrosol, oleuropein, oleocanthal, oleacein, ligstroside derivatives, tocopherols, phytosterols, triterpenes, flavonoids, and phenolic acids. These naturally occurring compounds have been associated with antioxidant activity, modulation of inflammatory pathways, cardiovascular support, cellular protection, oxidative stress reduction, metabolic regulation, neuroprotective effects, gastrointestinal health, and overall wellness.

Although olive oil itself has been extensively consumed as a food ingredient for centuries, increasing scientific attention has focused on the naturally occurring phenolic fraction present within olive oil. Unlike the lipid fraction, which primarily contributes caloric and structural properties, the phenolic compounds are believed to contribute many of the biological activities associated with high-quality olive oils. Numerous studies have demonstrated that olive oils exhibiting elevated concentrations of naturally occurring polyphenols generally possess greater oxidative stability, increased antioxidant capacity, enhanced resistance to degradation, and improved preservation of biologically active constituents when compared to oils containing lower phenolic concentrations.

The concentration of naturally occurring phenolic compounds within olive oil is influenced by numerous factors including olive cultivar, fruit maturity, harvesting methods, environmental conditions, irrigation practices, soil composition, climate, geographical origin, agricultural practices, extraction methods, processing conditions, and storage history. Certain olive-growing environments, including volcanic, mountainous, mineral-rich, semi-arid, coastal, biodynamic, regenerative, and organically cultivated regions, have been reported to produce olives containing elevated concentrations of naturally occurring phenolic compounds as a result of environmental stress responses and favorable cultivation conditions. Consequently, substantial variability exists among commercially available olive oils with respect to phenolic composition, antioxidant capacity, sensory characteristics, and biological activity.

Despite these recognized advantages, olive oil remains significantly underutilized as a primary carrier system within nutraceutical delivery platforms. Traditional nutraceutical formulations have generally relied upon capsules, softgels, liquid oils, tablets, or conventional powder formulations that frequently utilize inert excipients such as cellulose derivatives, starches, sugars, maltodextrins, or synthetic carriers. While these conventional carrier materials facilitate manufacturing and dosage form production, they contribute little or no intrinsic biological activity and often fail to exploit the therapeutic potential of naturally occurring bioactive lipids.

One significant challenge associated with incorporating olive oil into nutraceutical products arises from its physical characteristics. Olive oil exists as a liquid at ambient temperatures, making it inherently incompatible with numerous conventional solid dosage manufacturing processes. Direct incorporation of liquid oils into tablets, lozenges, powders, granules, chewable products, oral films, or similar dosage forms frequently results in poor flow characteristics, oil migration, phase separation, inconsistent ingredient distribution, reduced compressibility, structural instability, leakage, and diminished shelf life. These manufacturing limitations have historically restricted olive oil primarily to softgel capsules, bottled oils, or liquid emulsions.

Another significant limitation involves the chemical instability of many naturally occurring olive oil constituents. Polyphenols, hydroxytyrosol, oleuropein, oleocanthal, oleacein, tocopherols, and other antioxidant compounds may gradually degrade when exposed to oxygen, moisture, elevated temperatures, ultraviolet radiation, prolonged storage, mechanical processing, or repeated environmental stress. Oxidative degradation may reduce antioxidant capacity, alter flavor characteristics, diminish biological activity, and shorten product shelf life. Furthermore, conventional manufacturing operations including blending, compression, drying, granulation, milling, transportation, and packaging may contribute to progressive degradation of sensitive phenolic compounds if appropriate protective measures are not employed.

Preservation of naturally occurring phenolic compounds presents an even greater challenge for olive oils specifically selected because of their elevated polyphenol concentrations. While high-polyphenol olive oils may initially contain substantially greater concentrations of biologically active compounds than conventional olive oils, improper processing, encapsulation, formulation, or storage may significantly diminish these elevated concentrations before administration to a consumer. Accordingly, simply selecting an olive oil with a high initial phenolic concentration does not necessarily ensure delivery of a corresponding amount of bioactive compounds at the time of consumption.

Additional formulation challenges arise from the sensory properties of high-polyphenol olive oils. Elevated concentrations of oleocanthal, oleuropein, and related phenolic compounds may contribute pronounced bitterness, pungency, peppery sensations, throat irritation, and strong flavor characteristics that reduce consumer acceptance. Such sensory characteristics become particularly problematic in orally dissolving dosage forms including lozenges, chewable tablets, oral films, gummies, and similar products where prolonged residence within the oral cavity increases exposure of taste receptors to the olive oil. Consequently, maintaining high concentrations of naturally occurring phenolic compounds while simultaneously providing acceptable organoleptic characteristics represents a significant formulation challenge.

Microencapsulation technologies have been investigated for protecting various food ingredients, flavors, oils, vitamins, fragrances, pharmaceuticals, and nutraceutical compounds from environmental degradation. Conventional microencapsulation techniques, including spray drying, coacervation, fluidized-bed coating, freeze drying, extrusion, liposomal encapsulation, and related particle-forming processes, may improve handling characteristics by converting liquid materials into particulate forms. However, many existing encapsulation systems are designed primarily to improve manufacturability or protect general lipid ingredients rather than to preserve the naturally occurring phenolic profile of high-polyphenol olive oils throughout processing, formulation, storage, transportation, and administration. Furthermore, many encapsulation technologies have been developed without considering the unique chemical interactions, oxidation pathways, and sensory characteristics associated with phenolic-rich olive oils.

In addition to protecting the olive oil itself, there exists an increasing need for delivery platforms capable of simultaneously incorporating high-polyphenol olive oil with multiple nutraceutical active ingredients within a unified formulation. Modern consumers increasingly prefer comprehensive nutritional products containing combinations of vitamins, minerals, botanical extracts, probiotics, prebiotics, adaptogens, amino acids, peptides, functional fibers, phospholipids, omega fatty acids, and other health-promoting compounds rather than consuming numerous individual supplements. However, incorporation of such chemically diverse ingredients into a single formulation introduces additional formulation complexity because hydrophilic and lipophilic compounds frequently exhibit differing stability profiles, processing requirements, and release characteristics.

Existing nutraceutical formulations generally treat olive oil as a passive dietary ingredient or simple lipid carrier rather than as a multifunctional bioactive delivery matrix. Consequently, conventional formulations frequently fail to exploit the naturally occurring antioxidant systems present within high-polyphenol olive oil that may assist in stabilizing co-formulated nutraceutical ingredients. Likewise, many existing products fail to preserve the elevated phenolic composition that distinguishes premium high-polyphenol olive oils from conventional edible oils.

There further remains a need for delivery platforms capable of utilizing olive oils obtained from diverse cultivars, geographical regions, and agricultural systems while accommodating naturally occurring variations in phenolic composition. Such delivery platforms should be capable of utilizing olive oils derived from conventional, organic, biodynamic, regenerative, irrigated, rain-fed, coastal, mountainous, volcanic, mineral-rich, Mediterranean, semi-arid, and other cultivation environments while preserving the naturally occurring biochemical characteristics associated with each source.

Accordingly, there exists a continuing need for improved nutraceutical delivery technologies capable of selecting, stabilizing, preserving, protecting, formulating, and delivering high-polyphenol olive oils containing elevated concentrations of naturally occurring phenolic compounds throughout manufacturing, storage, transportation, and administration. There further exists a need for multifunctional delivery platforms that not only preserve the intrinsic biological properties of high-polyphenol olive oil but also facilitate the incorporation, stabilization, controlled release, and enhanced delivery of vitamins, minerals, botanical extracts, probiotics, prebiotics, peptides, amino acids, functional lipids, and other nutraceutical ingredients in convenient consumer-friendly dosage forms.

The present invention addresses these and other technical challenges by providing a comprehensive high-polyphenol olive oil nutraceutical delivery platform incorporating advanced encapsulation technologies, bioactive lipid delivery matrices, preservation methodologies, and manufacturing systems configured to maintain elevated phenolic concentrations while enabling formulation into a wide variety of solid, semi-solid, and other orally administrable nutraceutical dosage forms.

### SUMMARY OF THE INVENTION

The present invention relates to a comprehensive nutraceutical delivery platform, compositions, formulations, systems, and methods utilizing high-polyphenol olive oil as a multifunctional bioactive lipid matrix for the stabilization, preservation, formulation, and delivery of nutraceutical ingredients in a wide variety of oral dosage forms. The invention establishes an integrated delivery platform in which olive oil containing elevated concentrations of naturally occurring phenolic compounds functions not merely as a dietary ingredient or passive lipid carrier, but as a biologically active structural matrix that contributes antioxidant activity, stability enhancement, controlled release characteristics, improved compatibility among diverse nutraceutical ingredients, and enhanced delivery performance.

In one aspect, the invention provides a nutraceutical composition comprising microencapsulated high-polyphenol olive oil, one or more nutraceutical active ingredients, and one or more dosage-forming components, wherein the high-polyphenol olive oil forms a multifunctional lipid-based delivery matrix configured to preserve naturally occurring phenolic compounds while supporting incorporation of vitamins, minerals, botanical extracts, adaptogens, amino acids, peptides, probiotics, prebiotics, phospholipids, omega fatty acids, functional fibers, antioxidants, and other biologically active ingredients. The resulting delivery platform enables multiple nutraceutical ingredients to be incorporated into a unified formulation while maintaining structural stability, product integrity, and desirable organoleptic characteristics.

In another aspect, the invention provides a high-polyphenol olive oil delivery platform in which the olive oil possesses elevated concentrations of naturally occurring bioactive constituents including, without limitation, total polyphenols, hydroxytyrosol, tyrosol, oleuropein, oleocanthal, oleacein, ligstroside derivatives, tocopherols, phytosterols, flavonoids, phenolic acids, triterpenes, and combinations thereof. In various embodiments, the olive oil may possess total phenolic concentrations ranging from approximately 300 milligrams per kilogram to greater than approximately 3,000 milligrams per kilogram, although other phenolic concentrations may likewise be utilized depending upon the desired formulation characteristics and intended nutritional application. The invention recognizes that elevated phenolic concentrations contribute not only to the biological activity of the olive oil itself but also to the stability, antioxidant capacity, and functional performance of the overall nutraceutical composition.

In another aspect, the invention provides methods for selecting olive oils based upon predetermined biochemical characteristics, including phenolic concentration, antioxidant capacity, cultivar, geographical origin, environmental growing conditions, extraction method, harvest maturity, storage history, or combinations thereof. The selected olive oils may be obtained from any suitable agricultural system, including conventional, organic, biodynamic, regenerative, irrigated, rain-fed, Mediterranean, coastal, mountainous, semi-arid, volcanic, mineral-rich, or other cultivation environments. In certain embodiments, the olive oil may be derived from olives cultivated within volcanic terroirs or other environmentally stressed growing regions that naturally promote elevated concentrations of phenolic compounds and associated antioxidants.

In another aspect, the invention provides preservation methodologies configured to maintain naturally occurring phenolic compounds throughout harvesting, extraction, transportation, processing, encapsulation, blending, formulation, packaging, storage, distribution, and consumer administration. The preservation methodologies may reduce oxidative degradation, hydrolytic degradation, thermal degradation, photodegradation, or other environmental mechanisms capable of reducing the concentration or biological activity of naturally occurring olive oil constituents. The invention thereby seeks to preserve the biochemical profile of the selected high-polyphenol olive oil from production through final consumer use.

The invention further provides advanced encapsulation technologies configured to stabilize high-polyphenol olive oil while improving compatibility with solid and semi-solid nutraceutical dosage forms. In various embodiments, encapsulation may be achieved using one or more wall-forming or carrier systems selected from maltodextrins, gum arabic, modified starches, proteins, polysaccharides, hydrocolloids, cyclodextrins, liposomes, nanoemulsions, microemulsions, coacervates, biopolymer matrices, lipid nanoparticles, solid lipid particles, nanostructured lipid carriers, protein-lipid complexes, encapsulating fibers, or combinations thereof. Such encapsulation systems may be configured to protect sensitive phenolic compounds from environmental degradation while converting the liquid olive oil into stable particulate materials suitable for blending, compression, molding, extrusion, casting, granulation, freeze-drying, spray-drying, or other manufacturing processes.

In another aspect, the invention provides microencapsulated high-polyphenol olive oil particles comprising a lipid core and one or more surrounding protective layers configured to isolate the olive oil from oxygen, moisture, ultraviolet radiation, elevated temperatures, reactive compounds, and other environmental conditions capable of degrading phenolic constituents. The protective layer may comprise a single encapsulating wall or multiple concentric encapsulating layers having differing compositions, permeabilities, dissolution characteristics, or release profiles. In certain embodiments, the encapsulating structure provides immediate release, delayed release, sustained release, sequential release, site-specific release, moisture-triggered release, pH-responsive release, enzyme-responsive release, or combinations thereof.

In another aspect, the invention establishes a dosage-form-independent delivery platform wherein substantially the same high-polyphenol olive oil matrix may be incorporated into numerous ingestible formulations without fundamentally altering the underlying delivery architecture. Representative dosage forms include lozenges, fast-dissolving tablets, orally disintegrating tablets, chewable tablets, gummies, capsules, softgels, powders, granules, sachets, oral films, nutritional beverages, nutritional shots, suspensions, emulsions, functional confectionery products, meal supplements, nutritional bars, chewable gels, and other edible or orally administrable formulations. This dosage-form-independent architecture enables broad commercial applicability while preserving the structural and functional characteristics of the underlying lipid delivery platform.

In another aspect, the invention provides multifunctional nutraceutical compositions in which the microencapsulated high-polyphenol olive oil serves simultaneously as (i) a biologically active nutraceutical ingredient, (ii) a lipid carrier phase for lipophilic and amphiphilic compounds, (iii) a structural delivery matrix for hydrophilic and lipophilic nutraceutical ingredients, (iv) a stability-enhancing system that protects co-formulated ingredients against degradation, (v) a controlled-release medium, (vi) an antioxidant-rich biochemical environment, and (vii) a bioavailability-enhancing lipid phase configured to facilitate solubilization and absorption of nutraceutical compounds following administration.

The invention further provides formulations incorporating one or more nutraceutical ingredients selected from vitamins, minerals, botanical extracts, medicinal plant derivatives, adaptogens, probiotics, prebiotics, amino acids, peptides, enzymes, phospholipids, omega fatty acids, carotenoids, flavonoids, coenzymes, proteins, functional fibers, phytochemicals, antioxidants, nutraceutical complexes, medicinal mushrooms, fermentation products, marine-derived compounds, and combinations thereof. Such ingredients may be incorporated individually or in any desired combination while maintaining compatibility with the high-polyphenol olive oil delivery matrix.

In another aspect, the invention provides manufacturing methods configured to preserve elevated phenolic concentrations throughout formulation. Representative methods include selecting a high-polyphenol olive oil having predetermined biochemical characteristics, preparing an encapsulatable lipid phase, producing encapsulated olive oil particles utilizing one or more encapsulation technologies, blending the encapsulated particles with nutraceutical ingredients and formulation excipients, producing one or more oral dosage forms, and packaging the resulting products within protective storage systems configured to minimize oxygen exposure, moisture ingress, ultraviolet exposure, and other degradation pathways. The manufacturing methods may additionally include quality-control procedures for monitoring phenolic concentration, antioxidant activity, encapsulation efficiency, particle size distribution, moisture content, oxidative stability, dissolution behavior, and product shelf life.

In certain embodiments, the invention further provides quality-control systems for classifying olive oils according to predetermined biochemical profiles prior to formulation. Such classification may include measurement of total polyphenol concentration, hydroxytyrosol concentration, oleuropein concentration, oleocanthal concentration, antioxidant capacity, free fatty acid content, peroxide value, ultraviolet absorption characteristics, moisture content, or other measurable quality parameters. Olive oils meeting predetermined quality thresholds may be selected for formulation, thereby improving consistency among production batches while ensuring delivery of desired concentrations of naturally occurring bioactive compounds.

Another aspect of the invention provides formulations specifically configured to preserve desirable sensory characteristics while maintaining elevated phenolic concentrations. The delivery platform may reduce undesirable bitterness, pungency, throat irritation, excessive peppery characteristics, lipid mouthfeel, and other organoleptic limitations commonly associated with phenolic-rich olive oils through encapsulation, controlled release, flavor modulation, or combinations thereof. Consequently, the invention enables consumer-friendly dosage forms capable of delivering high concentrations of naturally occurring phenolic compounds without substantially compromising palatability.

The invention further establishes a scalable platform architecture adaptable to numerous nutraceutical, functional food, medical nutrition, and wellness applications. Because the delivery platform is based upon preservation and controlled delivery of high-polyphenol olive oil rather than a single formulation or ingredient combination, the underlying technology may be adapted to numerous health-support formulations addressing cardiovascular health, metabolic wellness, immune support, healthy aging, cognitive function, gastrointestinal health, inflammatory balance, cellular protection, sports nutrition, and general nutritional supplementation without departing from the principles of the invention.

Accordingly, the present invention provides a comprehensive high-polyphenol olive oil nutraceutical delivery platform that integrates olive oil selection, phenolic preservation, advanced encapsulation technologies, multifunctional lipid delivery matrices, controlled release systems, manufacturing methodologies, quality-control procedures, and versatile dosage-form architectures into a unified technology capable of preserving naturally occurring olive oil bioactives while facilitating incorporation of numerous nutraceutical ingredients into stable, consumer-friendly, and commercially scalable formulations. The invention thereby represents a significant advancement over conventional olive oil nutraceutical formulations by transforming high-polyphenol olive oil into a standardized, multifunctional delivery platform capable of preserving and delivering both the intrinsic biological benefits of premium olive oil and the complementary benefits of co-formulated nutraceutical ingredients.

### BRIEF DESCRIPTION OF THE ACCOMPANYING DRAWINGS

The accompanying drawings illustrate representative embodiments of the high-polyphenol olive oil nutraceutical delivery platform, compositions, formulations, encapsulation technologies, manufacturing systems, and methods disclosed herein. The drawings are provided for purposes of illustration and to facilitate understanding of various aspects of the invention and should not be construed as limiting the invention to the specific embodiments depicted. Like reference numerals generally refer to corresponding components throughout the several views.

FIG. 1 illustrates a schematic overview of a high-polyphenol olive oil nutraceutical delivery platform in which high-polyphenol olive oil is incorporated into a multifunctional lipid-based delivery matrix together with one or more nutraceutical active ingredients to produce a consumer-ready oral dosage form.

FIG. 2 illustrates a system architecture showing the integration of high-polyphenol olive oil, encapsulation technologies, nutraceutical ingredients, dosage-forming materials, and manufacturing processes into a unified nutraceutical delivery platform.

FIG. 3 illustrates an exemplary composition architecture in which a microencapsulated high-polyphenol olive oil matrix surrounds and supports multiple nutraceutical ingredients including vitamins, minerals, botanical extracts, probiotics, peptides, amino acids, and additional functional compounds.

FIG. 4 illustrates representative sources of high-polyphenol olive oil, including different olive cultivars, geographical regions, agricultural systems, harvesting conditions, and terroirs capable of producing olive oils having elevated naturally occurring phenolic concentrations.

FIG. 5 illustrates a representative biochemical composition of high-polyphenol olive oil including naturally occurring polyphenols, hydroxytyrosol, tyrosol, oleuropein, oleocanthal, oleacein, ligstroside derivatives, tocopherols, phytosterols, flavonoids, phenolic acids, triterpenes, and additional bioactive constituents.

FIG. 6 illustrates an exemplary olive oil selection and quality-control process in which olive oils are evaluated according to predetermined biochemical characteristics including total polyphenol concentration, antioxidant capacity, free fatty acid content, peroxide value, cultivar, geographical origin, and other quality parameters prior to formulation.

FIG. 7 illustrates an exemplary high-polyphenol olive oil microencapsulation architecture including a lipid core, one or more encapsulating layers, protective barrier materials, and optional controlled-release structures configured to preserve phenolic integrity.

FIG. 8 illustrates representative encapsulation technologies including spray drying, freeze drying, liposomal encapsulation, cyclodextrin inclusion complexes, nanoemulsions, microemulsions, coacervation, lipid nanoparticles, solid lipid particles, protein matrices, polysaccharide matrices, hydrocolloid systems, and combinations thereof.

FIG. 9 illustrates a representative phenolic preservation system configured to reduce oxidation, moisture exposure, thermal degradation, photodegradation, oxygen exposure, and other degradation pathways during harvesting, processing, encapsulation, formulation, storage, transportation, and administration.

FIG. 10 illustrates an exemplary manufacturing process for producing a nutraceutical composition comprising high-polyphenol olive oil, including olive oil selection, encapsulation, blending with nutraceutical ingredients, dosage-form production, quality assurance, and protective packaging.

FIG. 11 illustrates an exemplary cross-sectional view of a finished nutraceutical dosage form showing the distribution of microencapsulated high-polyphenol olive oil particles, nutraceutical active ingredients, binders, sweeteners, excipients, and structural matrix components.

FIG. 12 illustrates representative controlled-release mechanisms in which encapsulated high-polyphenol olive oil undergoes immediate release, delayed release, sustained release, sequential release, pH-responsive release, enzyme-responsive release, or moisture-triggered release following oral administration.

FIG. 13 illustrates representative oral dosage forms including lozenges, fast-dissolving tablets, orally disintegrating tablets, chewable tablets, gummies, capsules, oral films, powders, granules, sachets, nutritional beverages, nutritional shots, functional confectionery products, and related formulations incorporating the high-polyphenol olive oil delivery platform.

FIG. 14 illustrates an exemplary interaction between the released high-polyphenol olive oil lipid phase and co-formulated nutraceutical ingredients, demonstrating lipid-assisted dispersion, stabilization, and delivery of hydrophilic and lipophilic compounds.

FIG. 15 illustrates an exemplary storage and packaging system configured to preserve elevated phenolic concentrations through oxygen-reducing packaging, moisture barriers, ultraviolet protection, inert atmospheres, and temperature-controlled storage conditions.

FIG. 16 illustrates an exemplary quality monitoring system for evaluating encapsulation efficiency, particle size distribution, moisture content, oxidative stability, dissolution characteristics, phenolic concentration, and overall product quality throughout manufacturing and storage.

FIG. 17 illustrates an exemplary multifunctional lipid delivery matrix in which high-polyphenol olive oil functions simultaneously as a bioactive ingredient, lipid carrier, structural delivery matrix, antioxidant system, controlled-release medium, and stability-enhancing environment for co-formulated nutraceutical ingredients.

FIG. 18 illustrates an exemplary consumer administration sequence demonstrating oral administration, dosage-form dissolution, controlled release of encapsulated high-polyphenol olive oil, preservation of naturally occurring phenolic compounds, lipid-assisted transport of nutraceutical ingredients, and subsequent gastrointestinal delivery.

FIG. 19 illustrates an alternative embodiment utilizing multilayer encapsulation architectures, nested microcapsules, or hybrid encapsulation systems configured to provide enhanced protection and staged release of high-polyphenol olive oil and associated nutraceutical ingredients.

FIG. 20 illustrates an overall system diagram summarizing the integrated high-polyphenol olive oil nutraceutical delivery platform, including olive oil sourcing, quality selection, encapsulation, preservation technologies, formulation, manufacturing, dosage-form production, packaging, storage, and consumer administration.

### DETAILED DESCRIPTION OF THE INVENTION

Additional features and advantages of the invention will be set forth in the description which follows, and in part will be obvious from the description, or may be learned by the practice of the invention. These and other features of the present invention will become more fully apparent from the following description, or may be learned by the practice of the invention as set forth hereinafter.

The present invention relates to a comprehensive high-polyphenol olive oil-based nutraceutical delivery platform that transforms naturally occurring high-polyphenol olive oil from a conventional edible oil or passive lipid excipient into a multifunctional biologically active delivery matrix capable of preserving, stabilizing, protecting, formulating, and delivering one or more co-formulated nutraceutical ingredients. The invention integrates agricultural selection, biochemical characterization, quality evaluation, advanced encapsulation technologies, formulation engineering, controlled-release mechanisms, manufacturing process control, preservation methodologies, protective packaging systems, computerized quality management, and dosage-form adaptability into a unified technological ecosystem. Rather than relying solely on the nutritional value of olive oil, the invention utilizes the naturally occurring biochemical profile of carefully selected high-polyphenol olive oil—including elevated concentrations of hydroxytyrosol, tyrosol, oleuropein derivatives, oleocanthal, oleacein, ligstroside derivatives, tocopherols, phytosterols, flavonoids, phenolic acids, triterpenes, monounsaturated fatty acids, and additional naturally occurring antioxidant constituents—as an integral structural and functional component of the overall delivery platform.

In one aspect, the invention employs a comprehensive biochemical quality characterization architecture configured to objectively evaluate candidate olive oils prior to formulation. Olive oil batches are subjected to analytical characterization using chromatographic, spectroscopic, chemical, biochemical, or related analytical techniques to generate detailed biochemical profiles that include phenolic concentrations, antioxidant capacity, oxidative stability, fatty acid composition, peroxide value, moisture content, and additional quality parameters. The resulting analytical data are compared with predetermined manufacturing specifications to identify olive oils possessing desirable biochemical characteristics suitable for incorporation into the delivery platform. This objective analytical selection process enables manufacturers to standardize naturally variable agricultural raw materials while improving manufacturing consistency and batch-to-batch reproducibility.

Following quality characterization, the selected high-polyphenol olive oil undergoes encapsulation using one or more advanced encapsulation technologies designed to substantially preserve the naturally occurring biochemical profile throughout manufacturing, storage, transportation, and administration. Representative encapsulation methodologies include spray drying, freeze drying, liposomal encapsulation, cyclodextrin inclusion complexes, nanoemulsions, microemulsions, coacervation, lipid nanoparticles, solid lipid particles, hydrocolloid systems, protein matrices, polysaccharide encapsulation systems, multilayer encapsulation architectures, hybrid encapsulation technologies, and combinations thereof. In preferred embodiments, the encapsulated particles comprise a high-polyphenol olive oil core surrounded by one or more stabilization layers, barrier layers, protective coatings, and optional release-controlling layers configured to reduce exposure to oxygen, moisture, ultraviolet radiation, reactive oxygen species, volatile compounds, and other degradation-promoting environmental conditions.

The encapsulated high-polyphenol olive oil particles are subsequently incorporated into one or more nutraceutical formulations together with selected nutraceutical active ingredients. Representative co-formulated ingredients include vitamins, minerals, botanical extracts, medicinal plant derivatives, probiotics, prebiotics, amino acids, peptides, enzymes, phospholipids, omega fatty acids, functional fibers, medicinal mushroom extracts, proteins, carotenoids, flavonoids, marine-derived compounds, fermentation products, adaptogens, antioxidants, trace minerals, and additional nutraceutical materials. Unlike conventional formulations in which olive oil functions merely as a carrier or excipient, the encapsulated high-polyphenol olive oil particles establish localized antioxidant-rich lipid microenvironments distributed throughout the finished dosage form. These microenvironments contribute to preservation of oxidation-sensitive ingredients, improve formulation compatibility among chemically diverse nutraceutical compounds, and facilitate coordinated delivery following administration.

The invention is further characterized by exceptional dosage-form flexibility. The same encapsulated high-polyphenol olive oil delivery matrix may be incorporated into numerous orally administrable products including lozenges, chewable tablets, orally disintegrating tablets, fast-dissolving tablets, gummies, capsules, oral films, powders, granules, sachets, nutritional beverages, wellness shots, functional confectionery products, nutritional bars, multiparticulate formulations, and additional edible dosage forms. This dosage-form-independent platform enables manufacturers to develop multiple commercial products utilizing substantially the same underlying encapsulated delivery technology while modifying only the surrounding formulation architecture, excipient system, flavor profile, release characteristics, or consumer presentation.

A further aspect of the invention relates to configurable controlled-release delivery. The encapsulation architecture enables independent regulation of dosage-form disintegration, encapsulated particle exposure, encapsulation layer degradation, olive oil release, and subsequent physiological dispersion. Depending upon the selected encapsulation materials and formulation design, the system may provide immediate release, delayed release, sustained release, sequential release, staged release, pulsatile release, pH-responsive release, enzyme-responsive release, moisture-responsive release, site-specific release, or combinations thereof. Following release, the preserved high-polyphenol olive oil establishes a lipid-assisted delivery environment that facilitates coordinated dispersion and transport of lipophilic, amphiphilic, and hydrophilic nutraceutical ingredients while maintaining the naturally occurring antioxidant composition of the released olive oil.

The invention additionally incorporates preservation strategies extending throughout the complete commercial lifecycle of the product. Preservation begins with olive cultivation, harvesting, extraction, and analytical selection, continues through encapsulation, formulation, manufacturing, and packaging, and extends through warehousing, transportation, retail distribution, consumer storage, and administration. Protective packaging technologies may include oxygen-barrier systems, moisture-barrier materials, controlled-atmosphere packaging, inert-gas flushing, vacuum packaging, ultraviolet-resistant materials, multilayer barrier laminates, desiccants, oxygen scavengers, environmental monitoring technologies, smart packaging systems, and digital quality indicators configured to reduce cumulative degradation of naturally occurring phenolic compounds during commercial storage and distribution.

In preferred embodiments, manufacturing operations are coordinated through computer-implemented management systems that integrate biochemical characterization, manufacturing process control, environmental monitoring, batch traceability, inventory management, quality assurance, documentation, and production optimization. Manufacturing information may be collected from laboratory instruments, environmental sensors, analytical systems, encapsulation equipment, formulation systems, packaging lines, and quality-control devices. The resulting digital records enable comprehensive batch traceability while supporting automated manufacturing control, predictive process optimization, statistical quality management, laboratory information management systems, enterprise manufacturing execution systems, artificial intelligence-assisted process optimization, machine-learning quality analysis, cloud-based manufacturing databases, and digital production documentation.

Collectively, the invention establishes an integrated technological platform that unifies agricultural raw material selection, biochemical quality characterization, encapsulation engineering, formulation science, preservation technology, controlled-release delivery, digital manufacturing management, protective packaging, and consumer administration into a coordinated system centered around high-polyphenol olive oil as a multifunctional biologically active delivery matrix. By preserving the naturally occurring biochemical profile of premium high-polyphenol olive oil while enabling broad formulation compatibility, scalable manufacturing, configurable release characteristics, and long-term commercial stability, the invention provides a versatile platform suitable for development and manufacture of a wide variety of nutraceutical products while maintaining the structural, biochemical, and functional advantages associated with naturally occurring high-polyphenol olive oil.

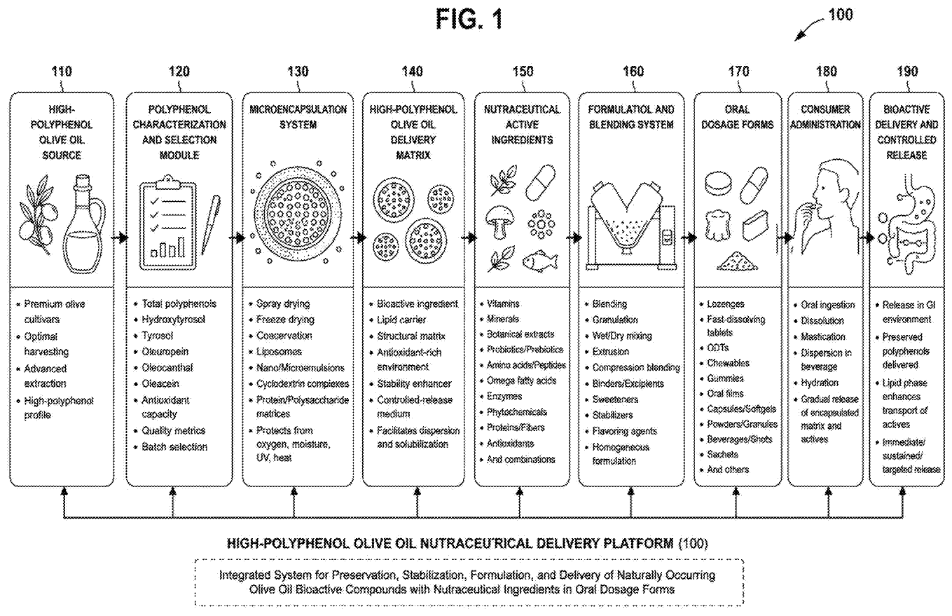

Referring initially to FIG. 1, there is illustrated a representative embodiment of a high-polyphenol olive oil nutraceutical delivery platform (100) configured to preserve, stabilize, formulate, and deliver naturally occurring olive oil bioactive compounds together with one or more nutraceutical ingredients in consumer-friendly oral dosage forms. The illustrated embodiment provides a conceptual overview of the integrated platform architecture and demonstrates the functional relationship among olive oil selection, biochemical characterization, encapsulation, formulation, dosage-form production, consumer administration, and biological delivery. Although illustrated as a sequential process for purposes of explanation, it should be understood that individual operations may be performed in various sequences, simultaneously, iteratively, or independently without departing from the scope of the invention.

As illustrated, the delivery platform (100) begins with a high-polyphenol olive oil source (110). The olive oil may be obtained from olives produced by any suitable cultivar, agricultural practice, geographical region, or extraction methodology. In various embodiments, the olive oil may originate from Mediterranean, coastal, mountainous, semi-arid, volcanic, mineral-rich, regenerative, biodynamic, organic, conventional, irrigated, rain-fed, or other agricultural systems. The invention is not limited to any particular olive variety or geographical origin and encompasses olive oils exhibiting naturally elevated concentrations of biologically active compounds regardless of production methodology.

Unlike conventional nutraceutical formulations that utilize generic edible olive oil, the present invention preferably utilizes olive oil selected according to predetermined biochemical characteristics rather than solely on commercial grade, geographical origin, or sensory attributes. Accordingly, the delivery platform includes a polyphenol characterization and selection module (120) configured to identify olive oils exhibiting elevated concentrations of naturally occurring bioactive compounds. Representative characteristics evaluated by the selection module include total polyphenol concentration, hydroxytyrosol concentration, tyrosol concentration, oleuropein content, oleocanthal concentration, oleacein concentration, ligstroside derivatives, tocopherol content, phytosterol concentration, antioxidant capacity, free fatty acid content, peroxide value, ultraviolet absorption characteristics, moisture content, oxidative stability, and combinations thereof.

In preferred embodiments, the olive oil selected by the characterization module (120) possesses total phenolic concentrations ranging from approximately 300 milligrams per kilogram to greater than approximately 3,000 milligrams per kilogram, although olive oils exhibiting lower or higher naturally occurring phenolic concentrations may likewise be utilized. The selection process may further include chemical analysis, chromatographic analysis, spectroscopic analysis, biochemical assays, antioxidant capacity measurements, laboratory testing, quality certification, or combinations thereof to ensure consistency among production batches.

Following selection of the desired olive oil, the platform proceeds to a microencapsulation system (130). The microencapsulation system converts the liquid high-polyphenol olive oil into stable particulate structures capable of incorporation into solid or semi-solid nutraceutical formulations. In various embodiments, encapsulation may be accomplished using spray drying, freeze drying, fluidized-bed coating, coacervation, liposomal encapsulation, cyclodextrin inclusion complexes, nanoemulsions, microemulsions, protein matrices, polysaccharide matrices, hydrocolloid systems, lipid nanoparticles, solid lipid particles, nanostructured lipid carriers, biopolymer encapsulation systems, or combinations thereof.

The encapsulation system (130) is configured to substantially isolate the olive oil from environmental conditions that promote degradation of naturally occurring phenolic compounds. In particular embodiments, the encapsulating materials reduce exposure to oxygen, moisture, ultraviolet radiation, elevated temperatures, reactive oxygen species, and other environmental factors capable of reducing antioxidant activity or degrading sensitive phenolic constituents. Consequently, the encapsulated particles provide improved storage stability while preserving the biochemical profile of the selected olive oil.

Following encapsulation, the resulting particles collectively form a high-polyphenol olive oil delivery matrix (140). Unlike conventional inert excipients commonly utilized in nutraceutical formulations, the delivery matrix (140) functions as a multifunctional bioactive system. More specifically, the delivery matrix simultaneously serves as: (i) a biologically active nutraceutical ingredient; (ii) a lipid carrier for lipophilic compounds; (iii) a structural carrier matrix for hydrophilic and lipophilic ingredients; (iv) an antioxidant-rich biochemical environment; (v) a stability-enhancing system; (vi) a controlled-release medium; and (vii) a lipid phase configured to facilitate dispersion, solubilization, and gastrointestinal delivery of co-formulated nutraceutical ingredients.

The delivery matrix (140) is subsequently combined with one or more nutraceutical active ingredients (150). Representative nutraceutical ingredients include vitamins, minerals, botanical extracts, medicinal plant derivatives, probiotics, prebiotics, adaptogens, amino acids, peptides, enzymes, omega fatty acids, phospholipids, carotenoids, flavonoids, proteins, medicinal mushroom extracts, functional fibers, antioxidants, phytochemicals, marine-derived compounds, fermentation products, or combinations thereof. The invention is not limited to any particular ingredient combination, and the delivery matrix may accommodate numerous formulation configurations depending upon the intended nutritional application.

The encapsulated olive oil particles and nutraceutical ingredients are introduced into a formulation and blending system (160) configured to produce substantially homogeneous nutraceutical compositions. The formulation system may include dry blending, wet blending, granulation, extrusion, compression blending, casting, molding, spray processing, continuous manufacturing, batch manufacturing, or other suitable formulation techniques. Sweeteners, binders, bulking agents, flavoring agents, lubricants, stabilizers, coloring agents, and additional excipients may also be incorporated to improve manufacturability, structural integrity, sensory characteristics, dissolution behavior, and shelf stability.

Following formulation, the resulting composition is processed into one or more oral dosage forms (170). Representative dosage forms include lozenges, fast-dissolving tablets, orally disintegrating tablets, chewable tablets, gummies, oral films, capsules, softgels, powders, granules, sachets, nutritional beverages, nutritional shots, suspensions, emulsions, functional confectionery products, nutritional bars, chewable gels, and other edible formulations. One significant advantage of the present invention is that substantially the same delivery matrix architecture may be incorporated into numerous dosage forms without fundamentally altering the underlying lipid-based delivery system.

During consumer use, the dosage form is administered through consumer administration (180). Depending upon the particular formulation, administration may involve oral dissolution, mastication, swallowing, dispersion within a beverage, suspension in liquid, or other ingestion methodologies. The dosage form gradually releases the encapsulated high-polyphenol olive oil together with the associated nutraceutical ingredients while maintaining protection of sensitive phenolic compounds until the desired stage of administration or digestion.

As further illustrated, administration results in bioactive delivery and controlled release (190). Upon hydration or exposure to saliva, gastric fluids, intestinal fluids, digestive enzymes, physiological pH conditions, or combinations thereof, the encapsulating structures gradually release the high-polyphenol olive oil. The liberated lipid phase facilitates dispersion and transport of lipophilic nutraceutical compounds while simultaneously delivering naturally occurring olive oil bioactive constituents including hydroxytyrosol, oleuropein, oleocanthal, oleacein, tocopherols, phytosterols, and other phenolic compounds. In various embodiments, the release profile may be immediate, sustained, delayed, staged, sequential, pH-responsive, enzyme-responsive, moisture-responsive, or combinations thereof.

An important feature illustrated in FIG. 1 is that the invention is directed not merely to a nutraceutical composition but to an integrated delivery platform in which olive oil selection, biochemical characterization, encapsulation, formulation, manufacturing, dosage-form production, and biological delivery operate as coordinated components of a unified system. The platform preserves elevated concentrations of naturally occurring phenolic compounds while enabling broad compatibility with diverse nutraceutical ingredients and numerous consumer dosage forms.

Accordingly, FIG. 1 provides a high-level conceptual representation of the present invention, illustrating how high-polyphenol olive oil (110) is selected through the characterization module (120), protected by the microencapsulation system (130), transformed into a multifunctional delivery matrix (140), combined with nutraceutical active ingredients (150), formulated by the blending system (160), processed into consumer dosage forms (170), administered by a consumer (180), and ultimately provides controlled delivery of preserved bioactive compounds through the delivery mechanism (190). Collectively, these components establish a comprehensive high-polyphenol olive oil nutraceutical delivery platform capable of preserving, protecting, and delivering naturally occurring olive oil bioactives together with a wide variety of nutraceutical ingredients.

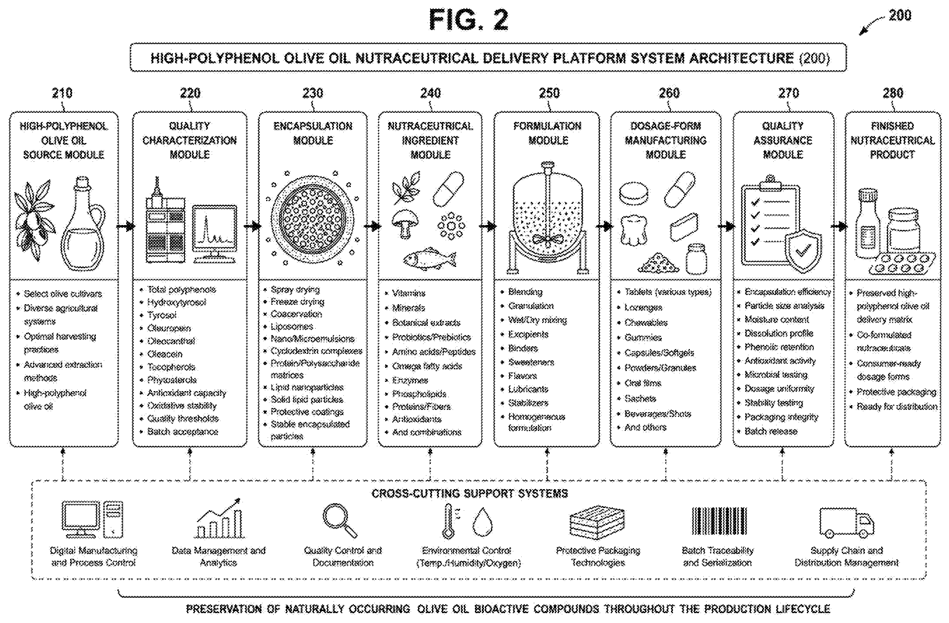

Referring now to FIG. 2, there is illustrated a representative system architecture (200) of the high-polyphenol olive oil nutraceutical delivery platform according to one embodiment of the present invention. The figure illustrates the coordinated interaction among ingredient sourcing, biochemical characterization, encapsulation technologies, formulation processes, manufacturing operations, quality assurance procedures, and dosage-form production that collectively produce a nutraceutical composition containing a preserved high-polyphenol olive oil delivery matrix. The illustrated architecture demonstrates that the invention extends beyond an individual formulation and instead encompasses an integrated manufacturing and delivery platform capable of preserving naturally occurring olive oil bioactives throughout the production lifecycle.

In the illustrated embodiment, the system architecture (200) includes a plurality of interconnected functional modules comprising a high-polyphenol olive oil source module (210), a quality characterization module (220), an encapsulation module (230), a nutraceutical ingredient module (240), a formulation module (250), a dosage-form manufacturing module (260), a quality assurance module (270), and a finished nutraceutical product (280). Although shown as sequentially connected modules for purposes of explanation, the invention is not limited to any specific process order, and individual operations may be performed simultaneously, iteratively, continuously, or in alternative manufacturing sequences.

The architecture begins with the high-polyphenol olive oil source module (210), which provides the primary lipid component utilized throughout the invention. The olive oil may be obtained from olives produced by any suitable cultivar or agricultural system, including conventional, organic, regenerative, biodynamic, irrigated, rain-fed, Mediterranean, volcanic, coastal, mountainous, mineral-rich, semi-arid, or other cultivation environments. In various embodiments, the selected olive oil possesses naturally elevated concentrations of bioactive phenolic compounds resulting from cultivar selection, environmental conditions, harvesting practices, or processing techniques.

The olive oil produced by the source module (210) is supplied to the quality characterization module (220). The quality characterization module evaluates one or more biochemical, chemical, physical, or nutritional characteristics of the olive oil before formulation. Representative quality parameters include total polyphenol concentration, hydroxytyrosol concentration, tyrosol concentration, oleuropein concentration, oleocanthal concentration, oleacein concentration, ligstroside derivatives, tocopherol content, phytosterol content, flavonoid concentration, triterpene concentration, antioxidant capacity, peroxide value, free fatty acid content, oxidative stability, ultraviolet absorption characteristics, moisture content, and combinations thereof.

In certain embodiments, the quality characterization module (220) compares measured biochemical characteristics against predetermined quality thresholds. Olive oils satisfying one or more predefined selection criteria may be accepted for formulation, whereas oils failing to satisfy such criteria may be rejected, reclassified, blended, or redirected for alternative applications. This quality-based selection process enables production consistency while ensuring preservation of elevated naturally occurring phenolic concentrations throughout commercial manufacturing.

Following characterization, the selected olive oil is transferred to the encapsulation module (230). The encapsulation module converts the liquid high-polyphenol olive oil into stable particulate materials suitable for incorporation into solid or semi-solid dosage forms. In various embodiments, the encapsulation module may employ spray drying, freeze drying, coacervation, liposomal encapsulation, cyclodextrin inclusion complex formation, nanoemulsion technology, microemulsion technology, fluidized-bed coating, biopolymer encapsulation, protein-based encapsulation, polysaccharide matrices, hydrocolloid systems, lipid nanoparticle formation, solid lipid particle technology, nanostructured lipid carriers, or combinations thereof.

The encapsulation module (230) is configured to produce encapsulated particles comprising a lipid core surrounded by one or more protective encapsulating layers. The encapsulating materials may reduce exposure of the olive oil to oxygen, moisture, heat, ultraviolet radiation, and reactive environmental species capable of degrading phenolic compounds. The resulting encapsulated particles therefore maintain improved biochemical stability while exhibiting favorable flowability, compressibility, dispersibility, and compatibility with conventional nutraceutical manufacturing processes.

The encapsulated olive oil particles produced by the encapsulation module (230) are combined with ingredients supplied by the nutraceutical ingredient module (240). The nutraceutical ingredient module may provide one or more biologically active compounds selected from vitamins, minerals, botanical extracts, medicinal plant derivatives, probiotics, prebiotics, amino acids, peptides, enzymes, phospholipids, omega fatty acids, functional fibers, proteins, antioxidants, flavonoids, carotenoids, medicinal mushroom extracts, fermentation products, marine-derived compounds, trace elements, nutraceutical complexes, and combinations thereof. The invention is not limited to any specific ingredient combination, and numerous formulation configurations may be implemented according to the desired nutritional application.

The encapsulated olive oil particles and nutraceutical ingredients are subsequently transferred to the formulation module (250). The formulation module combines the encapsulated particles with one or more formulation excipients including binders, sweeteners, bulking agents, lubricants, flavoring agents, stabilizers, coloring agents, anti-caking agents, compression aids, coating materials, disintegrants, and additional formulation components. Representative processing operations include dry blending, wet granulation, direct compression, extrusion, molding, casting, spray processing, continuous manufacturing, batch manufacturing, fluidized-bed processing, and related pharmaceutical or nutraceutical manufacturing techniques.

During formulation, the encapsulated high-polyphenol olive oil particles become substantially uniformly distributed throughout the formulation mixture, thereby establishing a multifunctional lipid-based delivery matrix. The lipid matrix serves not only as a biologically active ingredient but also as a structural carrier capable of supporting uniform dispersion of both hydrophilic and lipophilic nutraceutical compounds. In addition, naturally occurring antioxidant compounds within the olive oil contribute to preservation of sensitive co-formulated ingredients by reducing oxidative degradation during manufacturing and storage.

Following blending, the formulation is introduced into the dosage-form manufacturing module (260). The manufacturing module converts the blended composition into one or more finished dosage forms including lozenges, fast-dissolving tablets, orally disintegrating tablets, chewable tablets, gummies, capsules, powders, granules, sachets, oral films, nutritional beverages, nutritional shots, suspensions, emulsions, functional confectionery products, nutritional bars, chewable gels, or other orally administrable formulations. The invention is not limited to any particular dosage form and may be adapted to numerous consumer delivery formats without modifying the underlying lipid delivery architecture.

After dosage-form production, the resulting products are evaluated by the quality assurance module (270). The quality assurance module may perform one or more analytical procedures including determination of encapsulation efficiency, particle size distribution, dissolution characteristics, moisture content, microbial quality, compression strength, friability, oxidative stability, phenolic concentration, antioxidant activity, ingredient uniformity, shelf-life estimation, sensory evaluation, packaging integrity, and other quality-control measurements. In certain embodiments, products failing to satisfy predetermined quality specifications may be rejected or reprocessed before commercial distribution.

The completed dosage forms are subsequently produced as the finished nutraceutical product (280). The finished product incorporates the preserved high-polyphenol olive oil delivery matrix together with one or more nutraceutical active ingredients in a stable, consumer-ready formulation. In various embodiments, the finished product may be packaged within oxygen-reducing, moisture-resistant, ultraviolet-protective, vacuum-sealed, inert-gas-filled, temperature-controlled, or other protective packaging systems configured to preserve the naturally occurring biochemical profile of the encapsulated olive oil throughout storage, transportation, and commercial distribution.

One significant feature illustrated by FIG. 2 is the integration of multiple manufacturing operations into a unified delivery platform. Rather than treating olive oil as a simple nutritional additive, the illustrated architecture establishes an interconnected production system in which olive oil selection, biochemical characterization, encapsulation, formulation, manufacturing, quality assurance, and dosage-form production collectively function to preserve elevated concentrations of naturally occurring phenolic compounds while simultaneously supporting incorporation of numerous nutraceutical ingredients.

Another important aspect illustrated by the system architecture is the modular nature of the invention. Each functional module may be independently modified, expanded, substituted, automated, or integrated with additional manufacturing systems without departing from the principles of the invention. For example, alternative encapsulation technologies, additional quality-control procedures, automated formulation equipment, artificial intelligence-assisted manufacturing controls, continuous production systems, or advanced packaging technologies may be incorporated while preserving the overall functionality of the high-polyphenol olive oil delivery platform.

Accordingly, FIG. 2 provides a comprehensive representation of the manufacturing and formulation architecture underlying the present invention. Beginning with the high-polyphenol olive oil source module (210) and proceeding through the quality characterization module (220), encapsulation module (230), nutraceutical ingredient module (240), formulation module (250), dosage-form manufacturing module (260), quality assurance module (270), and finished nutraceutical product (280), the illustrated architecture demonstrates a coordinated platform capable of selecting, preserving, encapsulating, formulating, manufacturing, and delivering high-polyphenol olive oil while maintaining elevated concentrations of naturally occurring bioactive compounds and providing broad compatibility with diverse nutraceutical formulations.

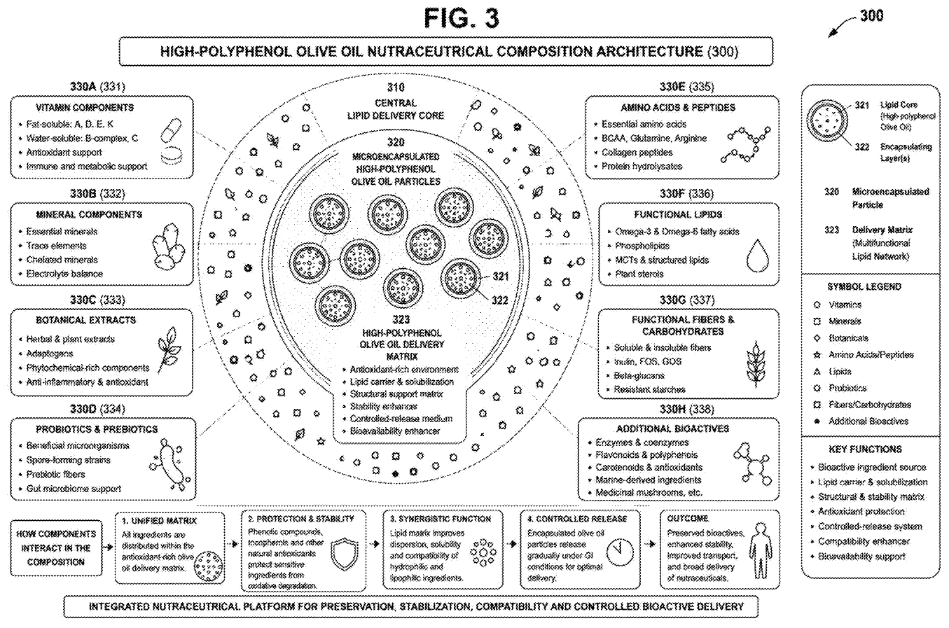

Referring now to FIG. 3, there is illustrated an exemplary composition architecture (300) of a nutraceutical formulation according to one embodiment of the present invention. The illustrated embodiment demonstrates the structural organization of a high-polyphenol olive oil delivery matrix and its functional relationship with multiple classes of nutraceutical active ingredients incorporated into a unified oral dosage composition. The figure is intended to conceptually illustrate the interaction among the various components of the formulation and should not be interpreted as limiting the invention to the specific geometric arrangement shown.

As illustrated, the composition architecture (300) comprises a central lipid delivery core (310) containing a plurality of microencapsulated high-polyphenol olive oil particles (320) distributed throughout a formulation matrix. The central lipid delivery core serves as the principal structural and functional component of the nutraceutical composition and establishes a biologically active matrix configured to support stabilization, preservation, controlled release, and delivery of multiple nutraceutical ingredients.

The microencapsulated high-polyphenol olive oil particles (320) each comprise a lipid core (321) surrounded by one or more encapsulating layers (322). The lipid core contains olive oil selected according to predetermined biochemical characteristics including elevated concentrations of naturally occurring polyphenols, hydroxytyrosol, tyrosol, oleuropein, oleocanthal, oleacein, ligstroside derivatives, tocopherols, phytosterols, flavonoids, triterpenes, phenolic acids, and additional antioxidant constituents. The encapsulating layer may comprise one or more wall-forming materials including maltodextrins, modified starches, gum arabic, proteins, polysaccharides, cyclodextrins, hydrocolloids, biopolymers, liposomes, lipid nanoparticles, nanoemulsions, microemulsions, coacervates, solid lipid particles, or combinations thereof.

In certain embodiments, the encapsulating layers (322) substantially isolate the lipid core from oxygen, moisture, ultraviolet radiation, elevated temperatures, reactive oxygen species, and other environmental conditions capable of degrading naturally occurring phenolic compounds. The encapsulated particles thereby preserve the biochemical profile of the selected high-polyphenol olive oil while simultaneously improving handling characteristics, flowability, compressibility, dispersibility, and compatibility with conventional nutraceutical manufacturing processes.

Collectively, the plurality of encapsulated particles (320) forms a high-polyphenol olive oil delivery matrix (323) extending throughout the central lipid delivery core (310). Unlike conventional inert excipients utilized in existing nutraceutical formulations, the delivery matrix (323) performs multiple biological and structural functions. More specifically, the delivery matrix simultaneously functions as a biologically active nutraceutical ingredient, a lipid carrier phase, a structural support matrix, an antioxidant-rich biochemical environment, a controlled-release medium, a stability-enhancing system, and a bioavailability-enhancing lipid network configured to facilitate transport of co-formulated nutraceutical compounds.

Surrounding the central lipid delivery core (310) are a plurality of nutraceutical ingredient regions (330A-330H). Although illustrated as discrete regions for purposes of explanation, the invention is not limited to any particular spatial arrangement, and the individual ingredients may instead be uniformly dispersed, layered, gradient-distributed, compartmentalized, concentrically arranged, or otherwise incorporated throughout the formulation.

A first nutraceutical region (330A) comprises one or more vitamin components (331). Representative vitamins include vitamins A, C, D, E, K, B-complex vitamins, and combinations thereof. The vitamin components may provide antioxidant activity, immune support, metabolic regulation, neurological support, bone health, cardiovascular support, or other nutritional functions. In certain embodiments, fat-soluble vitamins associate with the lipid phase provided by the high-polyphenol olive oil matrix, thereby improving dispersion and gastrointestinal transport.

A second nutraceutical region (330B) comprises one or more mineral components (332). Representative minerals include zinc, magnesium, selenium, calcium, chromium, potassium, iron, copper, manganese, iodine, molybdenum, and additional nutritionally beneficial trace elements. These mineral ingredients may be incorporated as inorganic salts, organic mineral complexes, chelated minerals, encapsulated minerals, or combinations thereof.

A third nutraceutical region (330C) comprises botanical extracts (333) selected from turmeric, ginger, garlic, ashwagandha, maca, ginseng, Rhodiola, Tribulus, green tea extract, elderberry, echinacea, medicinal mushroom extracts, or other plant-derived bioactive compounds. Such botanical ingredients may provide antioxidant, adaptogenic, immune-supporting, anti-inflammatory, metabolic, cardiovascular, cognitive, gastrointestinal, or other health-related benefits.

A fourth nutraceutical region (330D) comprises one or more probiotic and prebiotic components (334). Representative probiotic microorganisms include species belonging to the genera Lactobacillus, Bifidobacterium, Bacillus, Saccharomyces, or related beneficial microorganisms. Representative prebiotics include inulin, fructooligosaccharides, galactooligosaccharides, resistant starches, soluble fibers, and additional substrates capable of supporting beneficial gastrointestinal microbiota. In various embodiments, the antioxidant-rich lipid matrix assists in protecting sensitive probiotic organisms during manufacturing and storage.

A fifth nutraceutical region (330E) comprises amino acids and peptide components (335). Representative amino acids include essential amino acids, branched-chain amino acids, glutamine, arginine, glycine, taurine, citrulline, lysine, methionine, and combinations thereof. Peptide components may include collagen peptides, bioactive peptides, fermented peptides, protein hydrolysates, or other nutritionally beneficial peptide materials.

A sixth nutraceutical region (330F) comprises functional lipid components (336) including omega-3 fatty acids, omega-6 fatty acids, phospholipids, medium-chain triglycerides, conjugated linoleic acid, structured lipids, plant sterols, or combinations thereof. Such lipid ingredients may cooperate with the olive oil matrix to improve dispersion of lipophilic compounds while enhancing formulation stability.

A seventh nutraceutical region (330G) comprises functional fiber and carbohydrate components (337) including soluble fibers, insoluble fibers, resistant starches, beta-glucans, arabinoxylans, pectins, cellulose derivatives, gums, and additional dietary fibers capable of supporting gastrointestinal health, formulation integrity, or controlled-release performance.

An eighth nutraceutical region (330H) comprises additional functional bioactive compounds (338) including enzymes, flavonoids, carotenoids, coenzymes, antioxidants, phytochemicals, fermentation products, marine-derived ingredients, adaptogens, medicinal mushroom extracts, trace nutrients, or additional nutraceutical ingredients not specifically illustrated in the preceding regions. The invention therefore accommodates a virtually unlimited variety of nutraceutical combinations while preserving the underlying delivery platform architecture.

During manufacture, each of the nutraceutical ingredient regions (330A-330H) becomes incorporated into the central lipid delivery matrix (323). Rather than existing as isolated ingredients, the individual nutraceutical components interact with the antioxidant-rich lipid environment established by the encapsulated high-polyphenol olive oil particles. The naturally occurring phenolic compounds present within the olive oil may reduce oxidative degradation of oxidation-sensitive ingredients while improving overall formulation stability throughout manufacturing, storage, transportation, and administration.

In various embodiments, the lipid delivery matrix facilitates uniform distribution of hydrophilic, amphiphilic, and lipophilic compounds throughout the formulation. Lipophilic ingredients may partition into or associate with the lipid phase surrounding the encapsulated olive oil particles, whereas hydrophilic ingredients remain dispersed within the surrounding formulation matrix. Consequently, the invention provides compatibility among nutraceutical ingredients exhibiting substantially different chemical properties while maintaining formulation homogeneity.

Following oral administration, hydration of the formulation initiates gradual disintegration of the surrounding dosage matrix while preserving the structural integrity of the encapsulated olive oil particles until the desired release conditions are encountered. Depending upon the encapsulation technology employed, the olive oil may undergo immediate release, delayed release, sustained release, sequential release, enzyme-responsive release, moisture-responsive release, pH-responsive release, or combinations thereof. As the encapsulating structures gradually release the lipid core, the liberated olive oil establishes a localized lipid phase that facilitates dispersion, solubilization, and gastrointestinal transport of associated nutraceutical compounds.

An important aspect illustrated by FIG. 3 is that the high-polyphenol olive oil delivery matrix performs substantially more than a carrier function. The matrix actively contributes naturally occurring antioxidants, phenolic compounds, tocopherols, phytosterols, and other bioactive constituents while simultaneously providing structural support, stabilization, controlled release, lipid-assisted transport, and compatibility among multiple nutraceutical ingredients. This multifunctional architecture distinguishes the present invention from conventional formulations utilizing biologically inert excipients as carrier materials.

Another significant feature illustrated by the composition architecture is its scalability and adaptability. The number, type, concentration, spatial arrangement, and relative proportions of the nutraceutical ingredient regions (330A-330H) may be modified according to the desired nutritional application without fundamentally altering the underlying high-polyphenol olive oil delivery matrix. Accordingly, the illustrated architecture supports formulations intended for cardiovascular health, immune support, healthy aging, cognitive performance, metabolic wellness, gastrointestinal health, sports nutrition, cellular protection, inflammatory balance, or general nutritional supplementation.

Accordingly, FIG. 3 illustrates a comprehensive composition architecture in which the central lipid delivery core (310), the microencapsulated high-polyphenol olive oil particles (320), the multifunctional delivery matrix (323), and the surrounding nutraceutical ingredient regions (330A-330H) collectively establish an integrated nutraceutical platform capable of preserving naturally occurring olive oil bioactive compounds while supporting incorporation, stabilization, controlled release, and biological delivery of numerous nutraceutical ingredients within a single consumer-ready formulation.

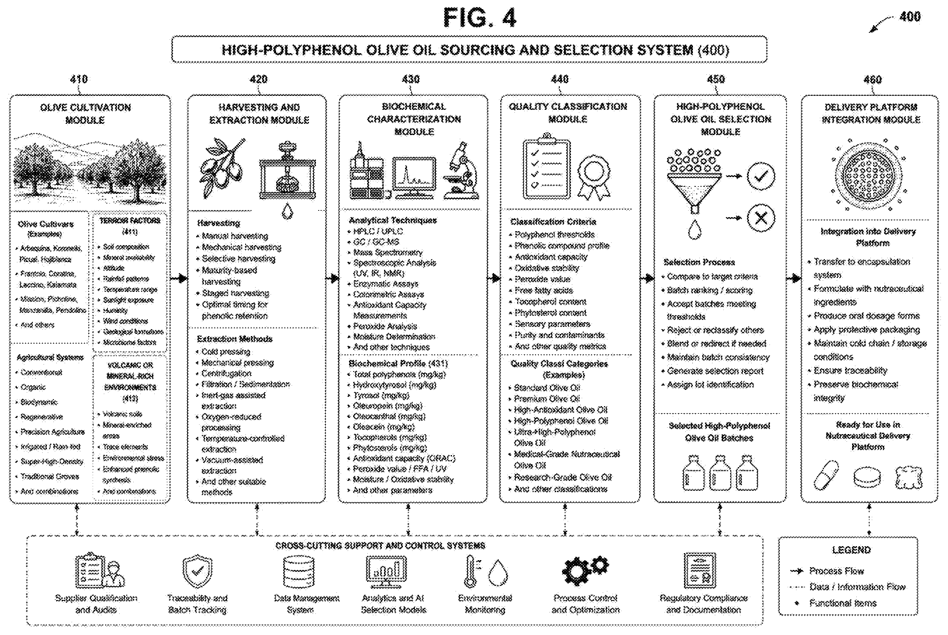

Referring now to FIG. 4, there is illustrated an exemplary high-polyphenol olive oil sourcing and selection system (400) configured to identify, evaluate, classify, and select olive oils exhibiting elevated concentrations of naturally occurring phenolic compounds for incorporation into the nutraceutical delivery platform of the present invention. Unlike conventional nutraceutical formulations that utilize commercially available olive oil without biochemical characterization, the present invention employs a structured selection methodology in which olive oils are evaluated according to predetermined biochemical, agricultural, geographical, and quality-related characteristics before formulation.

As illustrated, the sourcing and selection system (400) comprises an olive cultivation module (410), a harvesting and extraction module (420), a biochemical characterization module (430), a quality classification module (440), a high-polyphenol olive oil selection module (450), and a delivery platform integration module (460). Collectively, these modules establish a comprehensive quality-driven process for selecting olive oils possessing desirable biochemical characteristics while preserving the naturally occurring bioactive compounds responsible for the functional advantages of the present invention.

The process begins with the olive cultivation module (410), which represents the agricultural origin of the olives utilized for producing the selected olive oil. The invention is not limited to any particular olive cultivar and may utilize olives obtained from numerous commercially cultivated or regionally indigenous varieties. Representative cultivars include Arbequina, Koroneiki, Picual, Hojiblanca, Frantoio, Coratina, Leccino, Kalamata, Mission, Picholine, Manzanilla, Pendolino, Moraiolo, Ascolana, Barnea, Empeltre, and additional olive varieties capable of producing oils containing naturally occurring phenolic compounds.

In various embodiments, the olive cultivation module (410) encompasses agricultural systems including conventional farming, organic cultivation, biodynamic agriculture, regenerative agriculture, precision agriculture, irrigated orchards, rain-fed orchards, intensive cultivation systems, super-high-density orchards, traditional groves, and combinations thereof. The invention recognizes that cultivation methodology may significantly influence the biochemical composition of the resulting olive oil and therefore encompasses olive oils obtained from substantially any agricultural production system.

In certain embodiments, the olive cultivation module (410) further includes geographical and environmental characteristics collectively referred to as terroir factors (411). Representative terroir factors include soil composition, mineral availability, altitude, rainfall patterns, irrigation practices, annual temperature variations, sunlight exposure, humidity, seasonal environmental stress, wind conditions, geological formations, and microbiological soil characteristics. These environmental variables may individually or collectively influence synthesis and accumulation of naturally occurring phenolic compounds within the olive fruit.

In preferred embodiments, the invention contemplates olive oils derived from olives cultivated within volcanic or mineral-rich agricultural environments (412). Such cultivation regions may exhibit naturally elevated concentrations of minerals and trace elements resulting from volcanic geological activity or other mineral-enriched soil conditions. Environmental stress associated with these cultivation environments may stimulate production of naturally occurring antioxidant compounds, resulting in olive oils possessing elevated concentrations of hydroxytyrosol, oleuropein, oleocanthal, oleacein, and related phenolic constituents. Nevertheless, the invention is not limited to volcanic environments and encompasses any cultivation region capable of producing olive oils exhibiting desirable biochemical characteristics.

Following cultivation, the olives are processed by the harvesting and extraction module (420). The harvesting module may include manual harvesting, mechanical harvesting, selective harvesting, maturity-based harvesting, staged harvesting, or combinations thereof. In various embodiments, harvest timing may be selected according to olive maturity, color index, sugar content, oil yield, phenolic concentration, moisture content, antioxidant capacity, or combinations thereof. Selection of an appropriate harvest stage may contribute to preservation of naturally occurring phenolic compounds within the resulting olive oil.

The extraction operations performed by the harvesting and extraction module (420) may include cold pressing, mechanical pressing, centrifugation, filtration, sedimentation, inert-gas-assisted extraction, oxygen-reduced processing, temperature-controlled extraction, vacuum-assisted processing, or other oil extraction techniques suitable for preserving naturally occurring bioactive constituents. In preferred embodiments, extraction temperatures are maintained at levels that substantially minimize thermal degradation of phenolic compounds while maximizing retention of naturally occurring antioxidants.

The extracted olive oil is subsequently transferred to the biochemical characterization module (430). The biochemical characterization module performs one or more analytical procedures for determining the biochemical profile of the produced olive oil. Representative analytical techniques include chromatographic analysis, spectroscopic analysis, mass spectrometry, infrared analysis, ultraviolet spectroscopy, high-performance liquid chromatography (HPLC), gas chromatography, nuclear magnetic resonance spectroscopy, enzymatic assays, colorimetric assays, antioxidant capacity measurements, peroxide analysis, moisture determination, and additional analytical methodologies suitable for characterizing olive oil composition.

The biochemical characterization module (430) may determine one or more measurable characteristics including total polyphenol concentration, hydroxytyrosol concentration, tyrosol concentration, oleuropein concentration, oleocanthal concentration, oleacein concentration, ligstroside derivatives, flavonoid content, phenolic acid concentration, tocopherol concentration, phytosterol concentration, triterpene concentration, chlorophyll content, carotenoid concentration, antioxidant capacity, oxidative stability, peroxide value, free fatty acid content, iodine value, moisture content, ultraviolet absorption characteristics, or combinations thereof.

In certain embodiments, the biochemical characterization module generates a biochemical profile (431) uniquely identifying each production batch of olive oil. The biochemical profile may be digitally stored, printed, associated with batch records, encoded within manufacturing documentation, or incorporated into quality-control systems to enable traceability and production consistency.

Following biochemical characterization, the olive oil is evaluated by the quality classification module (440). The quality classification module compares the measured biochemical characteristics against one or more predetermined quality criteria. Representative classification categories may include standard olive oil, premium olive oil, high-antioxidant olive oil, high-polyphenol olive oil, ultra-high-polyphenol olive oil, medical-grade nutraceutical olive oil, research-grade olive oil, or additional quality classifications established according to biochemical measurements.

In one representative embodiment, the quality classification module identifies olive oils exhibiting total polyphenol concentrations within predetermined ranges, including concentrations greater than approximately 300 milligrams per kilogram, greater than approximately 500 milligrams per kilogram, greater than approximately 1,000 milligrams per kilogram, greater than approximately 2,000 milligrams per kilogram, greater than approximately 3,000 milligrams per kilogram, or other concentration ranges appropriate for the intended formulation. These values are exemplary only and should not be interpreted as limiting the scope of the invention.

Olive oils satisfying the predetermined selection criteria are transferred to the high-polyphenol olive oil selection module (450). This module identifies production batches suitable for incorporation into the nutraceutical delivery platform. Oils failing to satisfy the desired biochemical specifications may be rejected, blended with additional oils, redirected to alternative applications, subjected to additional processing, or otherwise managed according to predetermined manufacturing protocols.

The selected olive oil is subsequently supplied to the delivery platform integration module (460), where it becomes incorporated into the high-polyphenol olive oil delivery platform described throughout the present specification. The selected olive oil may undergo encapsulation, blending with nutraceutical ingredients, dosage-form production, packaging, storage, and consumer administration while preserving the elevated concentrations of naturally occurring phenolic compounds identified during the characterization process.

One significant advantage illustrated by FIG. 4 is that the present invention treats olive oil selection as an integral component of the nutraceutical delivery platform rather than merely as a raw material procurement step. By incorporating biochemical characterization and quality classification into the production architecture, the invention provides a reproducible and standardized methodology for manufacturing nutraceutical products containing consistently elevated concentrations of naturally occurring olive oil bioactive compounds.

Another important aspect illustrated by FIG. 4 is the recognition that olive oil quality extends beyond conventional commercial grading parameters. Rather than relying solely upon extra virgin classification or sensory evaluation, the present invention selects olive oil according to measurable biochemical characteristics directly related to antioxidant capacity and phenolic composition. This biochemical selection methodology enables formulation of nutraceutical products exhibiting improved consistency, biological activity, oxidative stability, and preservation of naturally occurring bioactive compounds.

Furthermore, the modular architecture illustrated in FIG. 4 permits incorporation of additional quality-control procedures, artificial intelligence-assisted selection algorithms, automated analytical instrumentation, digital traceability systems, blockchain-based batch verification, environmental monitoring systems, predictive quality models, or additional analytical technologies without departing from the principles of the invention.

Accordingly, FIG. 4 illustrates a comprehensive sourcing and selection architecture in which the olive cultivation module (410), harvesting and extraction module (420), biochemical characterization module (430), quality classification module (440), high-polyphenol olive oil selection module (450), and delivery platform integration module (460) collectively establish a standardized methodology for identifying, preserving, and utilizing high-polyphenol olive oils possessing elevated concentrations of naturally occurring bioactive compounds for incorporation into the nutraceutical delivery platform of the present invention.

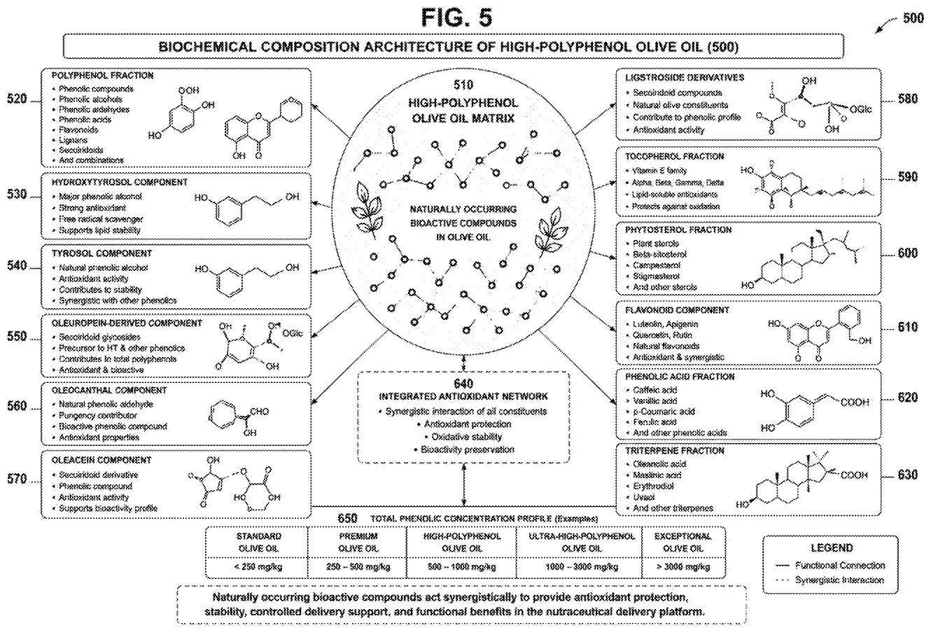

Referring now to FIG. 5, there is illustrated an exemplary biochemical composition architecture (500) of a representative high-polyphenol olive oil utilized in the nutraceutical delivery platform according to one embodiment of the present invention. The illustrated embodiment conceptually depicts the naturally occurring biochemical constituents present within the selected olive oil and demonstrates how these constituents collectively contribute to the multifunctional biological and structural characteristics of the delivery platform. Although individual biochemical components are illustrated as separate functional groups for purposes of explanation, it should be understood that the various constituents coexist within the olive oil and may interact synergistically to provide the overall properties of the invention.

As illustrated, the biochemical composition architecture (500) comprises a high-polyphenol olive oil matrix (510) that contains a plurality of naturally occurring bioactive compounds. Unlike conventional edible olive oils selected solely according to commercial grading, sensory evaluation, or geographic origin, the olive oil utilized in the present invention is preferably selected according to its naturally occurring biochemical profile, particularly its elevated concentration of phenolic compounds and antioxidant constituents.

The high-polyphenol olive oil matrix (510) contains a polyphenol fraction (520) representing one of the principal functional components of the invention. The polyphenol fraction may comprise one or more phenolic compounds naturally occurring within olive oil, including hydroxytyrosol, tyrosol, oleuropein derivatives, oleocanthal, oleacein, ligstroside derivatives, phenolic alcohols, phenolic aldehydes, phenolic acids, flavonoids, lignans, secoiridoids, and combinations thereof. The total concentration and relative proportions of these compounds may vary depending upon olive cultivar, agricultural practices, geographical origin, fruit maturity, extraction methodology, storage conditions, and environmental growing conditions.

In preferred embodiments, the polyphenol fraction (520) contributes significantly to the antioxidant capacity and oxidative stability of the selected olive oil. The naturally occurring phenolic compounds may assist in scavenging reactive oxygen species, reducing lipid oxidation, protecting co-formulated nutraceutical ingredients from oxidative degradation, and maintaining the biochemical integrity of the delivery platform throughout manufacturing, storage, transportation, and consumer administration.

The biochemical composition architecture further comprises a hydroxytyrosol component (530). Hydroxytyrosol represents one of the naturally occurring phenolic alcohols present within olive oil and is recognized for its antioxidant properties. In various embodiments, hydroxytyrosol contributes to preservation of lipid stability, reduction of oxidative degradation, enhancement of antioxidant capacity, and maintenance of product quality throughout the useful shelf life of the formulation.

The olive oil further includes a tyrosol component (540). Tyrosol is another naturally occurring phenolic alcohol that may contribute antioxidant activity and biochemical stability. The concentration of tyrosol may vary according to olive cultivar, extraction process, fruit maturity, environmental conditions, and storage history. The present invention encompasses olive oils containing naturally occurring tyrosol concentrations regardless of the specific source or production methodology.

The biochemical composition architecture additionally includes an oleuropein-derived component (550). Oleuropein and related secoiridoid derivatives naturally occur within olives and olive oil and contribute to the overall phenolic composition. During harvesting, extraction, storage, and processing, oleuropein may undergo enzymatic or chemical transformation into additional biologically active compounds while remaining part of the naturally occurring biochemical profile utilized by the present invention.

A further constituent comprises an oleocanthal component (560). Oleocanthal is a naturally occurring phenolic compound that contributes to the characteristic pungency associated with many premium olive oils. In preferred embodiments, preservation of naturally occurring oleocanthal during processing forms part of the overall objective of maintaining the biochemical integrity of the selected high-polyphenol olive oil throughout manufacture of the nutraceutical delivery platform.

The olive oil matrix (510) further contains an oleacein component (570). Oleacein represents another naturally occurring secoiridoid derivative that contributes to the antioxidant profile of olive oil. Together with hydroxytyrosol, tyrosol, oleuropein derivatives, and oleocanthal, oleacein forms part of the naturally occurring phenolic system that distinguishes high-polyphenol olive oils from conventional edible olive oils.

In addition to the principal phenolic constituents, the biochemical composition architecture includes ligstroside derivatives (580). Ligstroside and related derivatives may occur naturally within olive oil depending upon cultivar, maturation stage, extraction methodology, and storage conditions. These compounds further contribute to the biochemical diversity of the selected olive oil and may participate in the overall antioxidant system of the delivery platform.

The high-polyphenol olive oil matrix further comprises a tocopherol fraction (590). Tocopherols represent naturally occurring lipid-soluble antioxidant compounds that contribute to oxidative stability of the olive oil. Representative tocopherols include alpha-tocopherol, beta-tocopherol, gamma-tocopherol, delta-tocopherol, and naturally occurring mixtures thereof. The tocopherol fraction may cooperate with the phenolic constituents to reduce oxidation of both the olive oil and co-formulated nutraceutical ingredients.

The biochemical composition architecture additionally includes a phytosterol fraction (600) comprising naturally occurring plant sterols including beta-sitosterol, campesterol, stigmasterol, and additional sterol compounds. These naturally occurring constituents contribute to the biochemical profile of the selected olive oil while further distinguishing the oil from synthetic lipid carriers or biologically inert formulation excipients.

A flavonoid component (610) is also illustrated. Representative flavonoids may include luteolin, apigenin, quercetin, rutin, and other naturally occurring flavonoid compounds present within olive oil. Although generally present in lower concentrations than the principal secoiridoid-derived phenolics, these flavonoids contribute to the overall antioxidant composition and biochemical diversity of the selected olive oil.

The biochemical composition architecture further includes a phenolic acid fraction (620). Representative phenolic acids include caffeic acid, vanillic acid, syringic acid, p-coumaric acid, ferulic acid, gallic acid, and naturally occurring derivatives thereof. These compounds may contribute additional antioxidant properties and participate in the overall phenolic network present within the olive oil.

The olive oil additionally comprises a triterpene fraction (630) containing naturally occurring triterpenoid compounds including oleanolic acid, maslinic acid, erythrodiol, uvaol, and additional triterpenes. These constituents further contribute to the biochemical complexity of the selected olive oil and represent additional naturally occurring bioactive components preserved by the present invention.

Surrounding the various biochemical constituents is an integrated antioxidant network (640). The antioxidant network represents the cooperative interaction among the naturally occurring phenolic compounds, tocopherols, flavonoids, phytosterols, triterpenes, phenolic acids, and additional antioxidant molecules present within the olive oil. Rather than functioning independently, these naturally occurring constituents collectively contribute to preservation of the olive oil and may also assist in protecting co-formulated nutraceutical ingredients against oxidative degradation throughout manufacturing and storage.

In preferred embodiments, the high-polyphenol olive oil utilized in the invention exhibits total phenolic concentrations (650) ranging from approximately 300 milligrams per kilogram to greater than approximately 3,000 milligrams per kilogram. However, the invention is not limited to these exemplary concentration ranges, and olive oils exhibiting lower or higher naturally occurring phenolic concentrations may likewise be utilized provided that the selected olive oil possesses biochemical characteristics appropriate for the intended formulation.

One significant feature illustrated by FIG. 5 is that the present invention utilizes the naturally occurring biochemical profile of the selected olive oil as a defining structural characteristic of the delivery platform rather than relying solely upon the presence of olive oil as a lipid ingredient. The biochemical composition itself becomes an integral design parameter influencing selection, encapsulation, formulation, preservation, and ultimately biological delivery.

Another important aspect illustrated by FIG. 5 is the synergistic relationship among the naturally occurring constituents of the olive oil. Preservation of the complete biochemical profile—including the phenolic compounds, tocopherols, phytosterols, flavonoids, phenolic acids, triterpenes, and additional naturally occurring antioxidants—provides a multifunctional bioactive matrix capable of supporting formulation stability while delivering the intrinsic biological characteristics associated with premium high-polyphenol olive oils.

Accordingly, FIG. 5 illustrates a representative biochemical composition architecture in which the high-polyphenol olive oil matrix (510) contains the polyphenol fraction (520), hydroxytyrosol component (530), tyrosol component (540), oleuropein-derived component (550), oleocanthal component (560), oleacein component (570), ligstroside derivatives (580), tocopherol fraction (590), phytosterol fraction (600), flavonoid component (610), phenolic acid fraction (620), triterpene fraction (630), the integrated antioxidant network (640), and the total phenolic concentration profile (650). Collectively, these naturally occurring constituents establish the biochemical foundation of the high-polyphenol olive oil nutraceutical delivery platform and distinguish the present invention from formulations utilizing conventional olive oils or biologically inert lipid carriers.

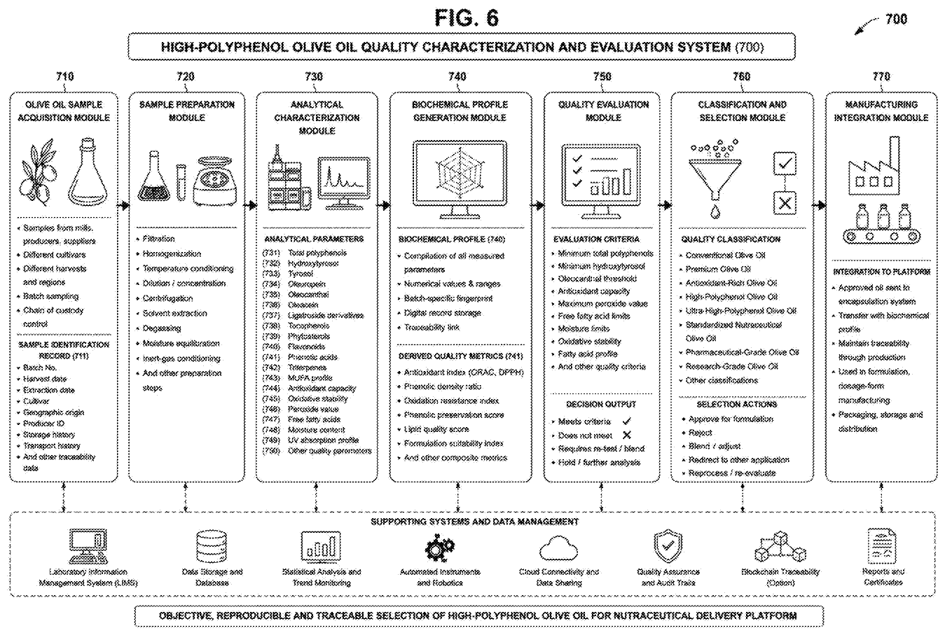

Referring now to FIG. 6, there is illustrated an exemplary high-polyphenol olive oil quality characterization and evaluation system (700) according to one embodiment of the present invention. The illustrated embodiment provides a comprehensive analytical and quality-control architecture configured to evaluate olive oil prior to incorporation into the nutraceutical delivery platform. Unlike conventional manufacturing processes that utilize olive oil primarily according to commercial grade or sensory evaluation, the present invention employs a structured biochemical characterization process to identify olive oils exhibiting desirable concentrations of naturally occurring bioactive compounds suitable for use within the delivery platform.

As illustrated, the quality characterization system (700) generally comprises an olive oil sample acquisition module (710), a sample preparation module (720), an analytical characterization module (730), a biochemical profile generation module (740), a quality evaluation module (750), a classification and selection module (760), and a manufacturing integration module (770). Collectively, these modules provide a standardized methodology for identifying olive oils possessing elevated naturally occurring phenolic concentrations while maintaining production consistency between manufacturing batches.

The process begins at the olive oil sample acquisition module (710). Representative olive oil samples may be obtained from production facilities, olive mills, commercial suppliers, growers, agricultural cooperatives, research organizations, certified producers, or directly from extraction facilities. The invention is not limited to any particular source, provided that the sampled olive oil can subsequently undergo biochemical characterization.

In various embodiments, each sampled olive oil is assigned a sample identification record (711) containing one or more identifiers including production batch number, harvest date, extraction date, olive cultivar, geographical origin, cultivation methodology, producer identification, storage history, transportation history, and other traceability information. Such records facilitate long-term manufacturing consistency and permit verification of biochemical characteristics throughout the production lifecycle.

The acquired sample is subsequently processed by the sample preparation module (720). Depending upon the analytical techniques employed, the sample preparation module may perform filtration, homogenization, temperature conditioning, dilution, centrifugation, solvent extraction, concentration adjustment, degassing, moisture equilibration, inert-gas conditioning, or additional preparatory procedures suitable for analytical evaluation. The invention is not limited to any particular sample preparation methodology.

Following preparation, the olive oil sample is introduced into the analytical characterization module (730). The analytical characterization module determines one or more measurable biochemical, chemical, physical, nutritional, or quality-related characteristics associated with the olive oil. Representative analytical procedures may include high-performance liquid chromatography (HPLC), ultra-high-performance liquid chromatography (UHPLC), gas chromatography (GC), gas chromatography-mass spectrometry (GC-MS), liquid chromatography-mass spectrometry (LC-MS), ultraviolet-visible spectroscopy, infrared spectroscopy, Raman spectroscopy, fluorescence spectroscopy, nuclear magnetic resonance spectroscopy, colorimetric assays, enzymatic assays, electrochemical analysis, antioxidant capacity measurements, or combinations thereof.

The analytical characterization module (730) may determine a plurality of measurable biochemical parameters including, without limitation:
(731) Total polyphenol concentration;
(732) Hydroxytyrosol concentration;
(733) Tyrosol concentration;
(734) Oleuropein concentration;
(735) Oleocanthal concentration;
(736) Oleacein concentration;
(737) Ligstroside derivatives;
(738) Tocopherol concentration;
(739) Phytosterol concentration;
(740) Flavonoid concentration;
(741) Phenolic acid concentration;
(742) Triterpene concentration;
(743) Monounsaturated fatty acid profile;
(744) Antioxidant capacity;
(745) Oxidative stability;
(746) Peroxide value;
(747) Free fatty acid content;
(748) Moisture content;
(749) Ultraviolet absorption characteristics; and
(750) Additional quality parameters appropriate for the intended formulation.

The invention is not limited to these analytical measurements, and additional biochemical, physical, or chemical analyses may likewise be incorporated.

The analytical results generated by the characterization module are transmitted to the biochemical profile generation module (740). This module compiles the measured analytical parameters into a comprehensive biochemical profile associated with the evaluated olive oil batch. In certain embodiments, the biochemical profile may comprise numerical values, concentration ranges, antioxidant indices, quality scores, multidimensional datasets, digital records, or combinations thereof.

The biochemical profile generation module may additionally calculate one or more derived quality metrics (741) based upon relationships among the measured analytical parameters. Representative derived metrics include antioxidant indices, phenolic density ratios, oxidation resistance indices, phenolic preservation scores, lipid quality scores, oxidative stability coefficients, formulation suitability indices, nutraceutical compatibility scores, or other composite measurements useful for manufacturing decision-making.

The generated biochemical profile is subsequently evaluated by the quality evaluation module (750). The quality evaluation module compares the measured biochemical characteristics with predetermined quality thresholds established for the intended nutraceutical formulation. In preferred embodiments, the evaluation process determines whether the olive oil possesses biochemical characteristics consistent with classification as a high-polyphenol olive oil suitable for incorporation into the delivery platform.

Representative evaluation criteria may include minimum total polyphenol concentration, minimum hydroxytyrosol concentration, acceptable oleocanthal concentration, acceptable antioxidant capacity, maximum peroxide value, acceptable free fatty acid content, acceptable moisture level, minimum oxidative stability, predetermined fatty acid composition, or combinations thereof. The evaluation criteria may vary according to the intended dosage form, storage conditions, manufacturing methodology, or desired commercial product characteristics.

In one representative embodiment, the quality evaluation module determines whether the olive oil possesses total phenolic concentrations greater than approximately 300 milligrams per kilogram. In alternative embodiments, higher quality thresholds including approximately 500 milligrams per kilogram, 750 milligrams per kilogram, 1,000 milligrams per kilogram, 2,000 milligrams per kilogram, or greater than approximately 3,000 milligrams per kilogram may be utilized depending upon the intended formulation. These concentration values are illustrative and should not be interpreted as limiting the invention.

Following evaluation, the olive oil is transferred to the classification and selection module (760). The classification module assigns the evaluated olive oil to one or more quality categories according to the measured biochemical profile. Representative classifications may include conventional olive oil, premium olive oil, antioxidant-rich olive oil, high-polyphenol olive oil, ultra-high-polyphenol olive oil, standardized nutraceutical olive oil, pharmaceutical-grade olive oil, research-grade olive oil, or additional quality categories established by the manufacturer.

In preferred embodiments, only olive oils satisfying predetermined biochemical criteria are approved for formulation within the nutraceutical delivery platform. Olive oils failing to satisfy one or more required quality parameters may be rejected, blended with additional production batches, redirected to alternative commercial applications, subjected to further processing, or retained for subsequent analytical evaluation.

The approved olive oil batches are supplied to the manufacturing integration module (770). The manufacturing integration module authorizes incorporation of the selected olive oil into downstream manufacturing processes including encapsulation, formulation, blending, dosage-form production, packaging, and commercial distribution. In certain embodiments, the associated biochemical profile accompanies the selected batch throughout manufacturing to ensure traceability and quality assurance.

One significant advantage illustrated by FIG. 6 is the incorporation of objective biochemical characterization into the manufacturing workflow. Rather than relying upon subjective sensory evaluation or conventional commercial grading systems, the present invention establishes reproducible analytical criteria that enable consistent selection of olive oils exhibiting elevated concentrations of naturally occurring phenolic compounds.

Another important feature illustrated by FIG. 6 is the ability to standardize naturally variable agricultural materials. Because olive oil composition varies according to cultivar, growing conditions, harvest timing, extraction methodology, and storage history, the illustrated characterization system enables manufacturers to produce nutraceutical formulations exhibiting substantially consistent biochemical characteristics despite natural variability among agricultural sources.

In certain embodiments, one or more modules of the quality characterization system may be automated. Automated liquid handling systems, robotic sample preparation equipment, computerized analytical instruments, machine-learning classification algorithms, laboratory information management systems (LIMS), digital traceability systems, blockchain verification systems, cloud-based quality databases, predictive quality models, or artificial intelligence-assisted analytical systems may be incorporated into the illustrated architecture without departing from the principles of the invention.

Furthermore, although FIG. 6 illustrates a representative analytical workflow, additional biochemical analyses, quality metrics, classification methodologies, statistical models, multivariate analyses, predictive algorithms, or formulation-specific evaluation criteria may be incorporated depending upon the desired commercial application, regulatory requirements, or manufacturing objectives.

Accordingly, FIG. 6 illustrates a comprehensive quality characterization architecture in which the olive oil sample acquisition module (710), sample preparation module (720), analytical characterization module (730), biochemical profile generation module (740), quality evaluation module (750), classification and selection module (760), and manufacturing integration module (770) collectively establish a standardized analytical framework for identifying, evaluating, selecting, and preserving high-polyphenol olive oils suitable for incorporation into the nutraceutical delivery platform of the present invention. The resulting characterization process enables consistent manufacturing of nutraceutical formulations while preserving the naturally occurring biochemical profile and functional advantages associated with premium high-polyphenol olive oils.

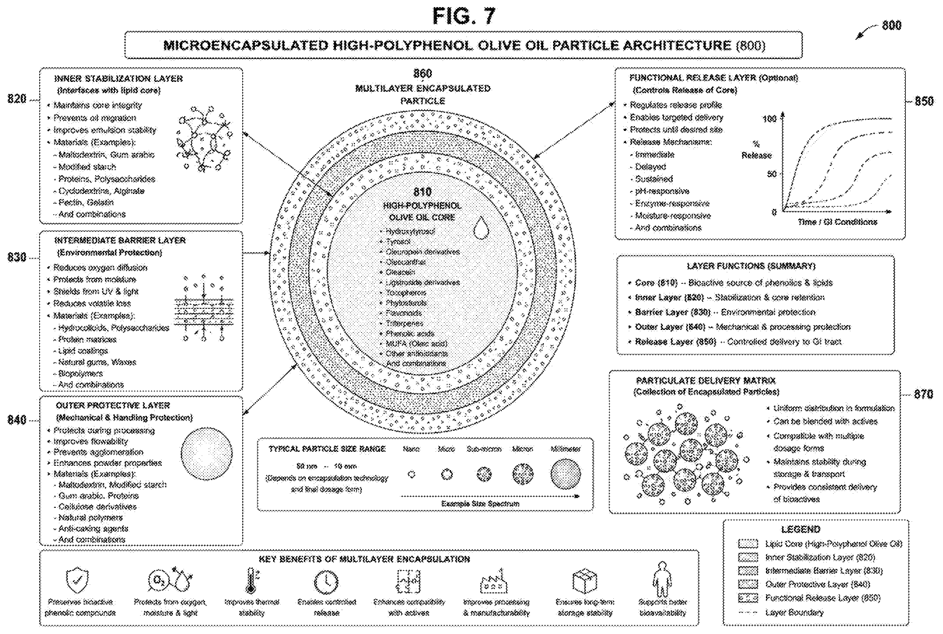

Referring now to FIG. 7, there is illustrated a representative microencapsulated high-polyphenol olive oil particle architecture (800) according to one embodiment of the present invention. The illustrated embodiment depicts an exemplary encapsulated particle configured to preserve the biochemical integrity of high-polyphenol olive oil while facilitating incorporation into solid, semi-solid, or other orally administrable nutraceutical dosage forms. Although FIG. 7 illustrates a generally spherical particle for purposes of explanation, the invention is not limited to any particular particle geometry, and the encapsulated structures may alternatively exhibit substantially spherical, ellipsoidal, ovoid, polygonal, irregular, multilobed, fibrous, plate-like, or other morphologies depending upon the encapsulation technology employed.

As illustrated, the encapsulated particle architecture (800) generally comprises a high-polyphenol olive oil core (810) surrounded by one or more protective encapsulating layers including an inner stabilization layer (820), an intermediate barrier layer (830), an outer protective layer (840), and an optional functional release layer (850). Collectively, these structural elements establish a multifunctional encapsulation system configured to preserve naturally occurring phenolic compounds while improving manufacturability, storage stability, and controlled delivery of the olive oil.

The high-polyphenol olive oil core (810) forms the central bioactive region of the encapsulated particle. The core contains olive oil selected according to the quality characterization procedures described previously and preferably possesses elevated concentrations of naturally occurring phenolic compounds. Representative bioactive constituents contained within the core include hydroxytyrosol, tyrosol, oleuropein derivatives, oleocanthal, oleacein, ligstroside derivatives, flavonoids, phenolic acids, tocopherols, phytosterols, triterpenes, monounsaturated fatty acids, and additional naturally occurring antioxidant compounds.

In preferred embodiments, the biochemical composition of the olive oil core (810) is substantially preserved throughout encapsulation, formulation, storage, transportation, and administration. Preservation of the naturally occurring phenolic profile enables the encapsulated particle to function not only as a lipid carrier but also as a biologically active delivery component capable of contributing antioxidant activity and supporting stabilization of co-formulated nutraceutical ingredients.

Surrounding the olive oil core is the inner stabilization layer (820). The inner stabilization layer directly interfaces with the lipid core and functions to maintain structural integrity of the encapsulated particle while minimizing migration of the liquid oil into surrounding formulation materials. In various embodiments, the inner stabilization layer may comprise maltodextrins, modified starches, gum arabic, proteins, polysaccharides, cyclodextrins, hydrocolloids, gelatin, alginates, pectins, cellulose derivatives, protein-lipid complexes, biopolymers, or combinations thereof.

In certain embodiments, the inner stabilization layer (820) further assists in maintaining uniform dispersion of the olive oil during encapsulation while reducing phase separation, oil leakage, particle aggregation, or instability during storage. The layer may additionally improve particle formation during spray drying, freeze drying, coacervation, fluidized-bed processing, extrusion, or other encapsulation techniques.

Positioned outwardly from the inner stabilization layer is the intermediate barrier layer (830). The intermediate barrier layer functions primarily as an environmental protection barrier configured to reduce diffusion of oxygen, moisture, ultraviolet radiation, volatile compounds, reactive oxygen species, and other degradation-promoting agents toward the olive oil core. By reducing environmental exposure, the barrier layer assists in preserving elevated concentrations of naturally occurring phenolic compounds during long-term storage and commercial distribution.

The intermediate barrier layer (830) may comprise one or more moisture-resistant, oxygen-resistant, or chemically protective materials including hydrocolloids, polysaccharides, protein matrices, biopolymer coatings, waxes, lipid coatings, shell materials, encapsulating polymers, natural gums, cellulose derivatives, modified starches, or multilayer combinations thereof. The composition and thickness of the barrier layer may vary according to the desired storage characteristics and intended release profile.

In certain embodiments, the intermediate barrier layer additionally reduces undesirable migration of volatile flavor compounds from the olive oil while minimizing absorption of moisture from the surrounding environment. Consequently, the encapsulated particle exhibits improved storage stability, enhanced handling characteristics, and reduced degradation of oxidation-sensitive bioactive constituents.

Surrounding the intermediate barrier layer is the outer protective layer (840). The outer protective layer provides mechanical protection during blending, granulation, compression, tablet formation, gummy production, capsule filling, packaging, transportation, and consumer handling. The outer layer may additionally improve particle flowability, reduce particle agglomeration, improve powder handling characteristics, and facilitate uniform distribution throughout the finished nutraceutical formulation.

Representative materials suitable for the outer protective layer include maltodextrins, gum arabic, modified starches, proteins, cellulose derivatives, polysaccharides, hydrocolloids, natural polymers, synthetic food-grade polymers, lipid coatings, mineral coatings, or combinations thereof. In various embodiments, the outer protective layer may further incorporate coloring agents, flavoring agents, sweeteners, anti-caking agents, lubricants, stabilizers, or additional excipients compatible with the intended dosage form.

In certain embodiments, the encapsulated particle further comprises an optional functional release layer (850). The functional release layer is configured to regulate release of the olive oil core following administration. Depending upon the selected encapsulation materials, the release layer may provide immediate release, delayed release, sustained release, sequential release, staged release, site-specific release, pH-responsive release, enzyme-responsive release, moisture-responsive release, temperature-responsive release, or combinations thereof.

The functional release layer (850) may comprise materials selected according to desired dissolution characteristics. Representative release-controlling materials include enteric coatings, pH-sensitive polymers, enzyme-degradable materials, moisture-sensitive matrices, biodegradable polymers, lipid coatings, hydrogel-forming materials, polysaccharide matrices, protein matrices, cellulose derivatives, or multilayer release structures. The invention is not limited to any particular release mechanism.

Collectively, the olive oil core (810), inner stabilization layer (820), intermediate barrier layer (830), outer protective layer (840), and functional release layer (850) establish a multilayer encapsulated particle (860) configured to protect and deliver high-polyphenol olive oil while preserving its naturally occurring biochemical profile.

In various embodiments, the encapsulated particles exhibit average particle diameters ranging from nanometer-scale particles to several millimeters depending upon the intended dosage form and encapsulation technology. Representative particle sizes may include approximately 50 nanometers to approximately 10 millimeters, although smaller or larger particle dimensions may likewise be utilized without departing from the scope of the invention.

Multiple encapsulated particles (860) may subsequently be blended together to form a particulate delivery matrix (870) distributed substantially uniformly throughout a nutraceutical composition. The particulate delivery matrix may be incorporated into lozenges, chewable tablets, fast-dissolving tablets, oral films, capsules, gummies, powders, granules, sachets, beverages, nutritional shots, functional confectionery products, and numerous additional dosage forms while preserving the structural integrity of the individual encapsulated particles.

During consumer administration, hydration of the dosage form gradually exposes the encapsulated particles to saliva, gastric fluids, intestinal fluids, digestive enzymes, physiological pH conditions, or combinations thereof. Depending upon the selected encapsulation architecture, the outer protective layer and functional release layer gradually degrade or dissolve, permitting controlled exposure of the intermediate barrier layer and eventual release of the olive oil core. This staged release process assists in preserving naturally occurring phenolic compounds until the desired stage of administration.

One important feature illustrated by FIG. 7 is that the encapsulated particle architecture performs multiple independent functions simultaneously. The particle protects the olive oil against environmental degradation, preserves naturally occurring phenolic compounds, improves manufacturing compatibility, enhances storage stability, reduces oxidation, facilitates controlled release, supports uniform formulation, and contributes to improved delivery of co-formulated nutraceutical ingredients.

Another significant aspect illustrated by FIG. 7 is that the multilayer architecture may be modified according to the desired commercial application. Individual layers may be omitted, duplicated, subdivided, combined, or supplemented with additional barrier layers, stabilizing layers, release-controlling layers, or functional coatings while preserving the overall principles of the invention. Likewise, different encapsulation materials may be selected for each layer depending upon the desired manufacturing process, dosage form, storage requirements, or release characteristics.

In certain embodiments, the encapsulated particles may additionally incorporate flavor-modifying agents, sweeteners, cooling agents, aromatic compounds, antioxidants, preservatives, oxygen scavengers, desiccant materials, colorants, identification markers, fluorescent tracers, edible pigments, nutritional markers, or additional functional additives without departing from the inventive concepts disclosed herein.

Accordingly, FIG. 7 illustrates a representative microencapsulated high-polyphenol olive oil particle architecture in which the high-polyphenol olive oil core (810), inner stabilization layer (820), intermediate barrier layer (830), outer protective layer (840), functional release layer (850), multilayer encapsulated particle (860), and particulate delivery matrix (870) collectively establish a robust encapsulation system configured to preserve naturally occurring olive oil bioactive compounds while providing enhanced stability, manufacturability, controlled release, and compatibility with a wide variety of nutraceutical formulations and oral dosage forms.

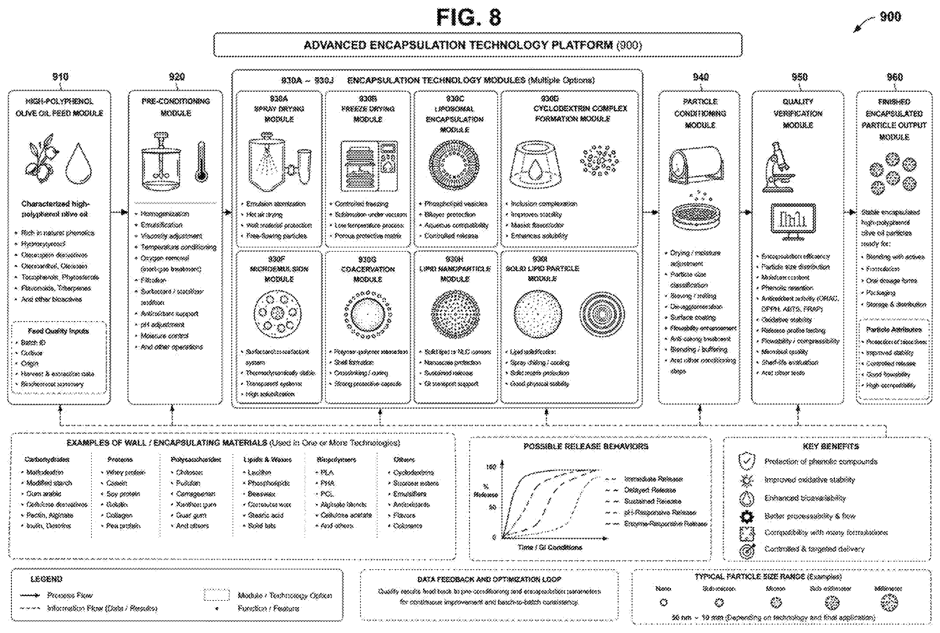

Referring now to FIG. 8, there is illustrated an exemplary advanced encapsulation technology platform (900) configured to produce stable encapsulated high-polyphenol olive oil particles suitable for incorporation into nutraceutical compositions according to various embodiments of the present invention. The illustrated embodiment demonstrates multiple encapsulation technologies that may be employed individually or in combination to preserve naturally occurring phenolic compounds, improve formulation stability, enhance manufacturability, regulate release characteristics, and facilitate incorporation of high-polyphenol olive oil into numerous oral dosage forms.

As illustrated, the advanced encapsulation technology platform (900) generally comprises a high-polyphenol olive oil feed module (910), a pre-conditioning module (920), a plurality of encapsulation technology modules (930A-930J), a particle conditioning module (940), a quality verification module (950), and a finished encapsulated particle output module (960). The various encapsulation technologies may operate independently, sequentially, simultaneously, or cooperatively depending upon the desired characteristics of the finished nutraceutical formulation.

The process begins with the high-polyphenol olive oil feed module (910). The feed module supplies previously characterized high-polyphenol olive oil selected according to the quality evaluation procedures described with reference to FIGS. 4 through 6. In preferred embodiments, the supplied olive oil possesses elevated concentrations of naturally occurring hydroxytyrosol, tyrosol, oleuropein derivatives, oleocanthal, oleacein, ligstroside derivatives, tocopherols, phytosterols, flavonoids, phenolic acids, triterpenes, and additional naturally occurring antioxidant compounds.

Prior to encapsulation, the supplied olive oil may optionally undergo processing within the pre-conditioning module (920). Representative pre-conditioning operations include homogenization, emulsification, viscosity adjustment, temperature conditioning, oxygen removal, inert-gas treatment, filtration, antioxidant supplementation, particle-size reduction, surfactant incorporation, stabilizer addition, pH adjustment, moisture control, or combinations thereof. The objective of the pre-conditioning stage is to prepare the olive oil for efficient encapsulation while minimizing degradation of naturally occurring phenolic compounds.

Following preparation, the olive oil is directed to one or more of the encapsulation technology modules (930A-930J). The invention is not limited to any single encapsulation methodology and instead encompasses a platform architecture capable of utilizing numerous encapsulation technologies depending upon the intended dosage form, manufacturing process, storage requirements, or desired release characteristics.

A first encapsulation technology module comprises a spray-drying module (930A). In this embodiment, the olive oil is dispersed within an aqueous encapsulating solution comprising one or more wall-forming materials including maltodextrins, modified starches, gum arabic, proteins, polysaccharides, hydrocolloids, cellulose derivatives, or combinations thereof. The resulting emulsion is atomized into a heated drying environment to produce free-flowing encapsulated particles containing the high-polyphenol olive oil within a protective particulate matrix.

A second encapsulation technology comprises a freeze-drying module (930B). The olive oil-containing formulation is subjected to controlled freezing followed by sublimation under reduced pressure, thereby producing porous encapsulated particles while reducing thermal exposure that could otherwise degrade oxidation-sensitive phenolic constituents.

A third encapsulation technology comprises a liposomal encapsulation module (930C). In this embodiment, phospholipid vesicles or liposomal structures are generated around the high-polyphenol olive oil, thereby producing lipid bilayer carriers capable of encapsulating and protecting the olive oil while improving compatibility with aqueous environments and facilitating controlled release following administration.

A fourth encapsulation technology comprises a cyclodextrin complex formation module (930D). Cyclodextrins or modified cyclodextrins may partially or completely surround selected olive oil constituents through inclusion complex formation, thereby improving oxidative stability, reducing volatility of aroma compounds, masking undesirable taste characteristics, and enhancing compatibility with solid dosage formulations.

A fifth encapsulation technology comprises a nanoemulsion formation module (930E). The nanoemulsion module produces emulsified olive oil droplets having characteristic dimensions generally within the nanometer range through high-pressure homogenization, ultrasonic processing, microfluidization, rotor-stator mixing, membrane emulsification, or related particle-size reduction technologies. Nanoemulsions may improve dispersion, stability, and delivery characteristics of the encapsulated olive oil.

A sixth encapsulation technology comprises a microemulsion module (930F). In this embodiment, the olive oil is incorporated into thermodynamically stable microemulsion systems comprising one or more surfactants, co-surfactants, aqueous phases, and lipid phases. The resulting structures provide stable encapsulation while facilitating subsequent incorporation into various nutraceutical formulations.

A seventh encapsulation technology comprises a coacervation module (930G). Oppositely charged polymers, proteins, polysaccharides, gelatin, gum arabic, alginates, pectins, chitosan, or other coacervating materials may be utilized to form protective encapsulating shells surrounding droplets of the high-polyphenol olive oil. Crosslinking, curing, or shell stabilization may subsequently be performed to improve mechanical strength and storage stability.

An eighth encapsulation technology comprises a lipid nanoparticle module (930H). In this embodiment, the olive oil is incorporated into lipid nanoparticles or nanostructured lipid carriers configured to improve protection of oxidation-sensitive phenolic compounds while enhancing gastrointestinal transport and controlled release characteristics.

A ninth encapsulation technology comprises a solid lipid particle module (930I). Solid lipid particles may be produced by cooling, crystallization, solvent evaporation, spray chilling, or related solidification processes to generate stable particulate carriers surrounding the olive oil. Such particles may provide enhanced physical stability and facilitate incorporation into compressed nutraceutical dosage forms.

A tenth encapsulation technology comprises a hybrid encapsulation module (930J). The hybrid module combines two or more encapsulation methodologies within a single production process. Representative combinations include spray-dried liposomes, nanoemulsion-containing microcapsules, cyclodextrin-coated lipid nanoparticles, multilayer coacervate systems, liposomal spray-dried particles, multilayer polysaccharide systems, or additional hybrid encapsulation architectures configured to optimize preservation of naturally occurring phenolic compounds.

Following encapsulation, the resulting particles are transferred to the particle conditioning module (940). Representative conditioning operations include drying, moisture adjustment, particle classification, sieving, de-agglomeration, milling, blending, coating, surface treatment, electrostatic stabilization, flow enhancement, anti-caking treatment, and additional post-processing operations suitable for preparing the encapsulated particles for subsequent formulation.

The conditioned particles are subsequently analyzed by the quality verification module (950). Representative quality evaluations include encapsulation efficiency, particle-size distribution, moisture content, oxygen permeability, dissolution profile, release kinetics, oxidative stability, phenolic retention, antioxidant activity, compressibility, flowability, particle morphology, surface integrity, microbial quality, and shelf-life evaluation. In preferred embodiments, the quality verification module confirms that the encapsulation process has substantially preserved the naturally occurring phenolic profile of the selected olive oil.

Encapsulated particles satisfying predetermined quality criteria are transferred to the finished encapsulated particle output module (960). The resulting particles may subsequently be blended with vitamins, minerals, botanical extracts, probiotics, prebiotics, amino acids, peptides, phospholipids, omega fatty acids, functional fibers, enzymes, medicinal plant extracts, medicinal mushroom extracts, or additional nutraceutical ingredients before incorporation into lozenges, chewable tablets, fast-dissolving tablets, gummies, capsules, powders, granules, oral films, sachets, beverages, nutritional shots, or other dosage forms.

One significant feature illustrated by FIG. 8 is the platform-based approach to encapsulation. Rather than restricting the invention to a single encapsulation process, the present invention encompasses numerous encapsulation technologies capable of preserving high-polyphenol olive oil while maintaining compatibility with a broad range of manufacturing processes and dosage forms. This flexibility enables manufacturers to select one or more encapsulation techniques according to product-specific performance requirements.

Another important aspect illustrated by FIG. 8 is that multiple encapsulation technologies may be combined to achieve enhanced protection of naturally occurring phenolic compounds. In certain embodiments, a first encapsulation technology may provide structural stability while a second encapsulation technology provides controlled release, oxidation resistance, moisture protection, flavor masking, or improved gastrointestinal delivery. Such hybrid architectures provide additional flexibility while preserving the biochemical integrity of the encapsulated olive oil.

Furthermore, the encapsulation technology platform may be integrated with automated manufacturing systems, continuous production lines, robotic process control, artificial intelligence-assisted process optimization, machine-learning process control, real-time analytical instrumentation, digital quality monitoring systems, or predictive manufacturing models without departing from the principles of the present invention.

Accordingly, FIG. 8 illustrates a comprehensive advanced encapsulation technology platform in which the high-polyphenol olive oil feed module (910), pre-conditioning module (920), the plurality of encapsulation technology modules (930A-930J), particle conditioning module (940), quality verification module (950), and finished encapsulated particle output module (960) collectively establish a versatile encapsulation architecture capable of preserving naturally occurring olive oil bioactive compounds while producing stable, high-quality encapsulated particles suitable for incorporation into a wide variety of nutraceutical delivery systems and oral dosage forms.

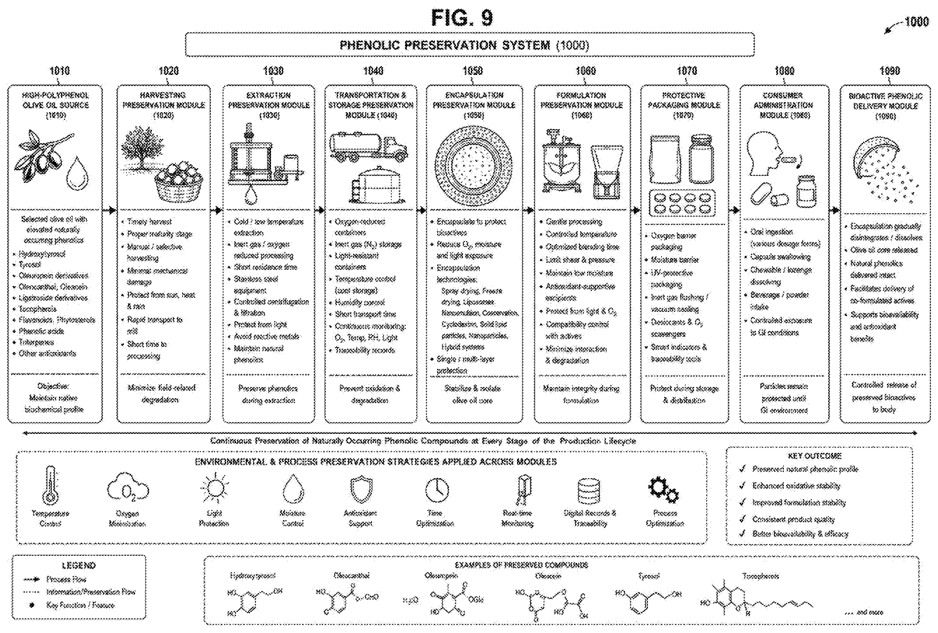

Referring now to FIG. 9, there is illustrated an exemplary phenolic preservation system (1000) according to one embodiment of the present invention. The illustrated embodiment depicts a comprehensive preservation architecture configured to substantially maintain the naturally occurring phenolic composition and antioxidant characteristics of high-polyphenol olive oil throughout harvesting, extraction, transportation, storage, encapsulation, formulation, manufacturing, packaging, distribution, and consumer administration. Unlike conventional nutraceutical manufacturing systems that primarily focus on formulation or encapsulation, the present invention establishes a coordinated preservation strategy extending across substantially the entire production lifecycle of the high-polyphenol olive oil.

As illustrated, the phenolic preservation system (1000) generally comprises a high-polyphenol olive oil source (1010), a harvesting preservation module (1020), an extraction preservation module (1030), a transportation and storage preservation module (1040), an encapsulation preservation module (1050), a formulation preservation module (1060), a protective packaging module (1070), a consumer administration module (1080), and a bioactive phenolic delivery module (1090). Collectively, these modules cooperate to preserve the naturally occurring biochemical profile of the selected olive oil from agricultural production through biological delivery.

The preservation process begins with the high-polyphenol olive oil source (1010). The selected olive oil possesses elevated concentrations of naturally occurring hydroxytyrosol, tyrosol, oleuropein derivatives, oleocanthal, oleacein, ligstroside derivatives, tocopherols, flavonoids, phytosterols, triterpenes, phenolic acids, and additional antioxidant constituents. The objective of the preservation system is to maintain these naturally occurring compounds throughout subsequent manufacturing operations while minimizing biochemical degradation.

Following olive cultivation, the olives enter the harvesting preservation module (1020). In various embodiments, harvesting operations are configured to minimize unnecessary mechanical damage, prolonged field exposure, excessive heat accumulation, oxidation, microbial deterioration, enzymatic degradation, or delays between harvesting and processing. Representative harvesting methodologies include manual harvesting, selective harvesting, mechanical harvesting, staged harvesting according to fruit maturity, rapid transport to extraction facilities, and environmentally controlled handling procedures.

In certain embodiments, the harvesting preservation module (1020) incorporates environmental monitoring systems configured to monitor ambient temperature, humidity, sunlight exposure, harvest duration, storage intervals, and transportation conditions before extraction. Such monitoring may assist in preserving naturally occurring phenolic compounds that could otherwise degrade following prolonged environmental exposure.

The harvested olives are subsequently processed by the extraction preservation module (1030). The extraction module is configured to recover olive oil while substantially preserving the naturally occurring biochemical composition of the oil. Representative extraction methodologies include cold pressing, mechanical extraction, centrifugation, inert-gas-assisted processing, oxygen-reduced extraction, filtration, sedimentation, temperature-controlled extraction, vacuum-assisted processing, and combinations thereof. The invention is not limited to any particular extraction technology provided that the extraction process substantially maintains the naturally occurring phenolic profile of the selected olive oil.

During extraction, the preservation module may minimize exposure of the olive oil to elevated temperatures, excessive oxygen, ultraviolet radiation, prolonged processing times, reactive metals, or additional degradation-promoting conditions. By limiting exposure to such environmental factors, the extracted olive oil retains higher concentrations of naturally occurring phenolic compounds suitable for subsequent encapsulation.

Following extraction, the olive oil enters the transportation and storage preservation module (1040). The transportation and storage module maintains the biochemical quality of the olive oil prior to encapsulation and formulation. Representative preservation measures include storage within oxygen-reduced containers, inert-gas-filled vessels, light-resistant containers, ultraviolet-protective packaging, temperature-controlled storage environments, humidity-controlled storage facilities, stainless-steel storage vessels, vacuum storage systems, or combinations thereof.

In certain embodiments, the transportation and storage preservation module continuously monitors one or more environmental parameters including storage temperature, oxygen concentration, moisture content, relative humidity, transportation duration, vibration exposure, ultraviolet exposure, container integrity, and storage atmosphere composition. These parameters may be recorded within quality-control databases to provide traceability and verification of storage conditions.

The preserved olive oil is subsequently introduced into the encapsulation preservation module (1050). The encapsulation module surrounds the olive oil with one or more protective layers configured to substantially reduce diffusion of oxygen, moisture, volatile compounds, ultraviolet radiation, reactive oxygen species, and other degradation-promoting agents. Representative encapsulation technologies include spray drying, freeze drying, liposomal encapsulation, nanoemulsions, microemulsions, cyclodextrin inclusion complexes, coacervation, lipid nanoparticles, solid lipid particles, protein matrices, hydrocolloid systems, polysaccharide encapsulation, biopolymer coatings, or combinations thereof.

The encapsulation preservation module (1050) may produce single-layer or multilayer encapsulated particles as previously described with reference to FIG. 7. In preferred embodiments, the encapsulated particles substantially isolate the olive oil from the surrounding formulation environment until administration while preserving the naturally occurring antioxidant profile.

Following encapsulation, the particles are processed within the formulation preservation module (1060). The formulation module incorporates the encapsulated high-polyphenol olive oil into one or more nutraceutical dosage forms while minimizing mechanical stress, excessive compression forces, prolonged heat exposure, moisture uptake, oxidative degradation, or undesirable chemical interactions with additional formulation ingredients. Representative formulation processes include direct compression, granulation, molding, extrusion, casting, capsule filling, gummy production, oral film formation, beverage preparation, sachet filling, powder blending, and related nutraceutical manufacturing operations.

In preferred embodiments, formulation parameters including processing temperature, residence time, compression force, moisture content, blending intensity, mixing duration, and environmental atmosphere may be optimized to reduce degradation of naturally occurring phenolic compounds during manufacturing.

Following formulation, the completed dosage forms are transferred to the protective packaging module (1070). The packaging module protects the formulated nutraceutical composition during commercial storage and distribution. Representative packaging systems include blister packaging, moisture-resistant containers, oxygen-barrier pouches, multilayer laminate packaging, vacuum-sealed containers, inert-gas-filled containers, desiccant-containing packages, ultraviolet-resistant containers, amber containers, foil packaging, multilayer polymer packaging, or combinations thereof.

In certain embodiments, the protective packaging module further incorporates oxygen scavengers, moisture scavengers, humidity indicators, freshness indicators, temperature indicators, tamper-evident features, smart packaging sensors, near-field communication devices, radio-frequency identification tags, digital tracking systems, or additional monitoring technologies configured to maintain and verify product quality throughout distribution.

Upon consumer use, the formulation enters the consumer administration module (1080). Depending upon the dosage form, administration may involve oral dissolution, chewing, swallowing, dispersion within a liquid, ingestion of capsules, consumption of beverages, or additional administration methods. During administration, the encapsulated olive oil remains substantially protected until exposure to predetermined physiological conditions including saliva, gastric fluids, intestinal fluids, digestive enzymes, physiological pH, or combinations thereof.

Following administration, the encapsulated particles proceed to the bioactive phenolic delivery module (1090). The delivery module gradually releases the preserved olive oil while maintaining substantially intact concentrations of naturally occurring hydroxytyrosol, tyrosol, oleuropein derivatives, oleocanthal, oleacein, ligstroside derivatives, tocopherols, flavonoids, phytosterols, triterpenes, phenolic acids, and additional antioxidant constituents. Controlled release of the preserved lipid phase further facilitates delivery of co-formulated nutraceutical ingredients contained within the dosage form.

One significant advantage illustrated by FIG. 9 is that preservation of naturally occurring phenolic compounds is addressed as a continuous manufacturing objective rather than an isolated encapsulation process. Each stage of the production lifecycle contributes to maintaining the biochemical integrity of the selected olive oil, thereby improving product consistency and preserving the functional characteristics associated with premium high-polyphenol olive oils.

Another important feature illustrated by FIG. 9 is the integration of multiple preservation mechanisms operating simultaneously. Agricultural selection, harvesting practices, extraction procedures, storage conditions, encapsulation technologies, formulation controls, packaging systems, and controlled physiological release collectively establish a comprehensive preservation architecture that substantially minimizes cumulative degradation of naturally occurring phenolic compounds.

In various embodiments, one or more modules of the phenolic preservation system may further incorporate computerized monitoring systems, artificial intelligence-assisted process optimization, predictive degradation models, digital quality records, blockchain traceability systems, environmental sensors, continuous analytical instrumentation, automated manufacturing controls, cloud-based manufacturing databases, or machine-learning quality optimization algorithms without departing from the principles of the invention.

Although FIG. 9 illustrates a representative preservation workflow, individual preservation modules may be omitted, duplicated, reordered, subdivided, or supplemented with additional preservation technologies depending upon the desired manufacturing process, commercial application, storage requirements, regulatory environment, or product characteristics while remaining within the scope of the present invention.

Accordingly, FIG. 9 illustrates a comprehensive phenolic preservation architecture in which the high-polyphenol olive oil source (1010), harvesting preservation module (1020), extraction preservation module (1030), transportation and storage preservation module (1040), encapsulation preservation module (1050), formulation preservation module (1060), protective packaging module (1070), consumer administration module (1080), and bioactive phenolic delivery module (1090) collectively establish an integrated preservation system configured to maintain the naturally occurring biochemical profile of high-polyphenol olive oil from agricultural production through biological delivery, thereby enhancing stability, consistency, and functional performance of the nutraceutical delivery platform.

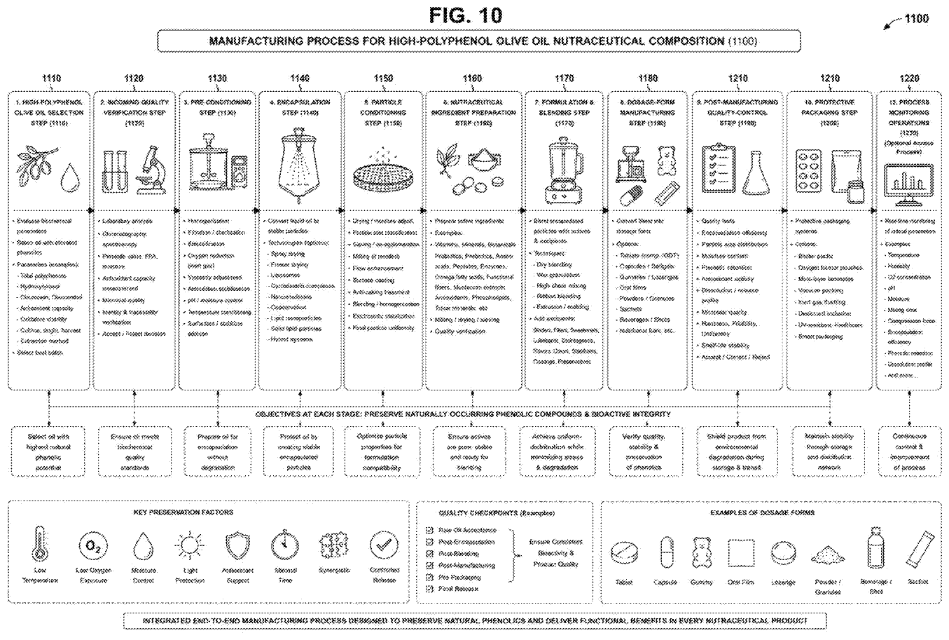

Referring now to FIG. 10, there is illustrated an exemplary manufacturing process (1100) for producing a nutraceutical composition comprising a high-polyphenol olive oil delivery platform according to one embodiment of the present invention. The illustrated process demonstrates a representative sequence of manufacturing operations beginning with selection of a high-polyphenol olive oil and concluding with production of a finished nutraceutical dosage form. Although illustrated as a sequential workflow for purposes of explanation, one skilled in the art will appreciate that individual operations may be performed simultaneously, iteratively, continuously, or in alternative process sequences without departing from the principles of the present invention.

The manufacturing process (1100) begins with a high-polyphenol olive oil selection step (1110). During this operation, olive oil is selected according to one or more predetermined biochemical quality criteria. Representative selection parameters include total polyphenol concentration, hydroxytyrosol concentration, tyrosol concentration, oleuropein content, oleocanthal concentration, oleacein concentration, ligstroside derivatives, tocopherol concentration, phytosterol concentration, antioxidant capacity, oxidative stability, peroxide value, free fatty acid content, moisture content, geographical origin, cultivar, agricultural practices, harvest maturity, extraction methodology, or combinations thereof. In preferred embodiments, the selected olive oil possesses elevated concentrations of naturally occurring phenolic compounds suitable for incorporation into the delivery platform.

Following selection, the olive oil undergoes an incoming quality verification step (1120). Representative verification procedures include laboratory testing, chromatographic analysis, spectroscopic analysis, antioxidant capacity measurement, peroxide value determination, free fatty acid analysis, moisture analysis, ultraviolet absorption testing, microbial evaluation, identity verification, batch traceability confirmation, or additional quality-control procedures appropriate for the intended commercial product. Olive oil batches satisfying predetermined quality specifications proceed to subsequent manufacturing operations, whereas nonconforming batches may be rejected, blended, reclassified, or redirected for alternative applications.

The verified olive oil is subsequently introduced into a pre-conditioning step (1130). Representative pre-conditioning operations include homogenization, filtration, emulsification, oxygen reduction, inert-gas treatment, viscosity adjustment, particle-size reduction, antioxidant stabilization, temperature conditioning, surfactant incorporation, pH adjustment, moisture control, or combinations thereof. The objective of the pre-conditioning step is to prepare the olive oil for encapsulation while minimizing degradation of naturally occurring phenolic compounds.

Following pre-conditioning, the olive oil undergoes an encapsulation step (1140). The encapsulation process converts the liquid olive oil into stable particulate structures suitable for incorporation into solid or semi-solid nutraceutical formulations. Representative encapsulation technologies include spray drying, freeze drying, coacervation, liposomal encapsulation, cyclodextrin inclusion complexes, nanoemulsions, microemulsions, lipid nanoparticles, solid lipid particles, nanostructured lipid carriers, hydrocolloid systems, protein matrices, polysaccharide matrices, biopolymer encapsulation systems, fluidized-bed coating, or combinations thereof. In preferred embodiments, the encapsulation process substantially preserves the naturally occurring phenolic composition of the selected olive oil while improving storage stability and manufacturability.

The encapsulated particles are then subjected to a particle conditioning step (1150). Representative conditioning operations include drying, moisture adjustment, particle classification, sieving, de-agglomeration, milling, coating, blending, flow enhancement, electrostatic stabilization, anti-caking treatment, particle surface modification, or combinations thereof. Particle conditioning produces encapsulated materials exhibiting suitable physical characteristics for subsequent formulation processes.

Following conditioning, the encapsulated olive oil particles are transferred to a nutraceutical ingredient preparation step (1160). In this operation, one or more nutraceutical active ingredients are prepared for formulation with the encapsulated olive oil. Representative ingredients include vitamins, minerals, botanical extracts, probiotics, prebiotics, amino acids, peptides, enzymes, phospholipids, omega fatty acids, functional fibers, medicinal mushroom extracts, fermentation products, antioxidants, carotenoids, flavonoids, trace minerals, proteins, adaptogens, marine-derived compounds, or combinations thereof. Individual ingredients may optionally undergo milling, particle-size adjustment, drying, encapsulation, sieving, blending, or quality verification before incorporation into the formulation.

The conditioned encapsulated olive oil particles and prepared nutraceutical ingredients are subsequently combined during a formulation and blending step (1170). Representative formulation operations include dry blending, wet blending, granulation, extrusion, direct compression blending, continuous mixing, batch mixing, high-shear mixing, low-shear blending, ribbon blending, fluidized-bed blending, casting, molding, or combinations thereof. Formulation excipients including binders, sweeteners, lubricants, disintegrants, fillers, stabilizers, flavoring agents, coloring agents, preservatives, anti-caking agents, coating materials, and additional formulation components may likewise be incorporated according to the intended dosage form.

During the formulation step, the encapsulated high-polyphenol olive oil particles become substantially uniformly distributed throughout the formulation mixture, thereby establishing a multifunctional lipid delivery matrix capable of supporting structural stability, controlled release, antioxidant preservation, and biological delivery of co-formulated nutraceutical ingredients.

Following blending, the formulated composition proceeds to a dosage-form manufacturing step (1180). Depending upon the intended commercial product, the formulation may be processed into lozenges, fast-dissolving tablets, orally disintegrating tablets, chewable tablets, gummies, oral films, capsules, powders, granules, sachets, nutritional beverages, nutritional shots, suspensions, emulsions, functional confectionery products, nutritional bars, chewable gels, or additional orally administrable dosage forms. Representative manufacturing operations include tablet compression, capsule filling, molding, extrusion, casting, film formation, gummy deposition, spray drying, freeze drying, or combinations thereof.

The manufactured dosage forms subsequently undergo a post-manufacturing quality-control step (1190). Representative quality evaluations include measurement of encapsulation efficiency, particle-size distribution, dosage uniformity, tablet hardness, friability, dissolution profile, moisture content, antioxidant activity, phenolic retention, oxidative stability, microbial quality, compression characteristics, sensory evaluation, shelf-life testing, packaging integrity, or combinations thereof. Products satisfying predetermined quality specifications advance to packaging, whereas nonconforming products may undergo corrective processing, rejection, or additional evaluation.

Following quality verification, the dosage forms are processed by a protective packaging step (1200). Representative packaging systems include blister packs, moisture-resistant containers, oxygen-barrier pouches, multilayer laminate packaging, foil pouches, vacuum-sealed packages, inert-gas-filled containers, ultraviolet-resistant containers, desiccant-containing packages, smart packaging systems, temperature-controlled containers, or combinations thereof. The protective packaging is configured to reduce degradation of naturally occurring phenolic compounds during commercial storage and transportation.

Following packaging, the completed products enter a finished product storage and distribution step (1210). Storage conditions may be controlled to minimize exposure to elevated temperatures, excessive humidity, ultraviolet radiation, oxygen, mechanical stress, or prolonged environmental exposure. Distribution may occur through conventional retail channels, healthcare providers, nutraceutical suppliers, pharmacies, direct-to-consumer channels, online distribution networks, or additional commercial distribution systems.

In certain embodiments, one or more manufacturing operations illustrated in FIG. 10 may be performed using automated production equipment. Automated weighing systems, robotic material handling systems, continuous manufacturing lines, programmable logic controllers, industrial process control systems, artificial intelligence-assisted manufacturing algorithms, machine-learning process optimization systems, cloud-connected manufacturing databases, real-time quality monitoring systems, or digital manufacturing execution systems may be incorporated to improve process consistency and production efficiency.

The manufacturing process may further incorporate one or more process monitoring operations (1220) distributed throughout the workflow. Representative monitored parameters include processing temperature, humidity, oxygen concentration, particle-size distribution, mixing time, compression force, encapsulation efficiency, moisture content, antioxidant capacity, phenolic retention, dissolution characteristics, microbial quality, viscosity, formulation homogeneity, and additional process variables relevant to the preservation of the high-polyphenol olive oil delivery platform.

One significant feature illustrated by FIG. 10 is that preservation of the naturally occurring phenolic profile is integrated throughout the manufacturing process rather than being confined solely to the encapsulation stage. Selection of the olive oil, incoming quality verification, pre-conditioning, encapsulation, formulation, dosage-form production, quality assurance, packaging, storage, and distribution collectively contribute to maintaining elevated concentrations of naturally occurring phenolic compounds within the finished nutraceutical product.

Another important aspect illustrated by FIG. 10 is the manufacturing flexibility provided by the present invention. The same underlying high-polyphenol olive oil delivery matrix may be incorporated into numerous dosage forms using different manufacturing technologies while preserving the structural and functional characteristics of the encapsulated olive oil. Consequently, manufacturers may produce diverse commercial products utilizing substantially the same underlying delivery platform.

Although FIG. 10 illustrates one representative manufacturing workflow, individual manufacturing operations may be omitted, repeated, reordered, subdivided, supplemented with additional processing steps, or combined according to the desired formulation, dosage form, commercial application, production capacity, regulatory requirements, or manufacturing technology without departing from the principles of the present invention.

Accordingly, FIG. 10 illustrates a comprehensive manufacturing process in which the high-polyphenol olive oil selection step (1110), incoming quality verification step (1120), pre-conditioning step (1130), encapsulation step (1140), particle conditioning step (1150), nutraceutical ingredient preparation step (1160), formulation and blending step (1170), dosage-form manufacturing step (1180), post-manufacturing quality-control step (1190), protective packaging step (1200), finished product storage and distribution step (1210), and optional process monitoring operations (1220) collectively establish an integrated manufacturing methodology configured to preserve naturally occurring phenolic compounds while producing stable, consumer-ready nutraceutical compositions incorporating the high-polyphenol olive oil delivery platform.

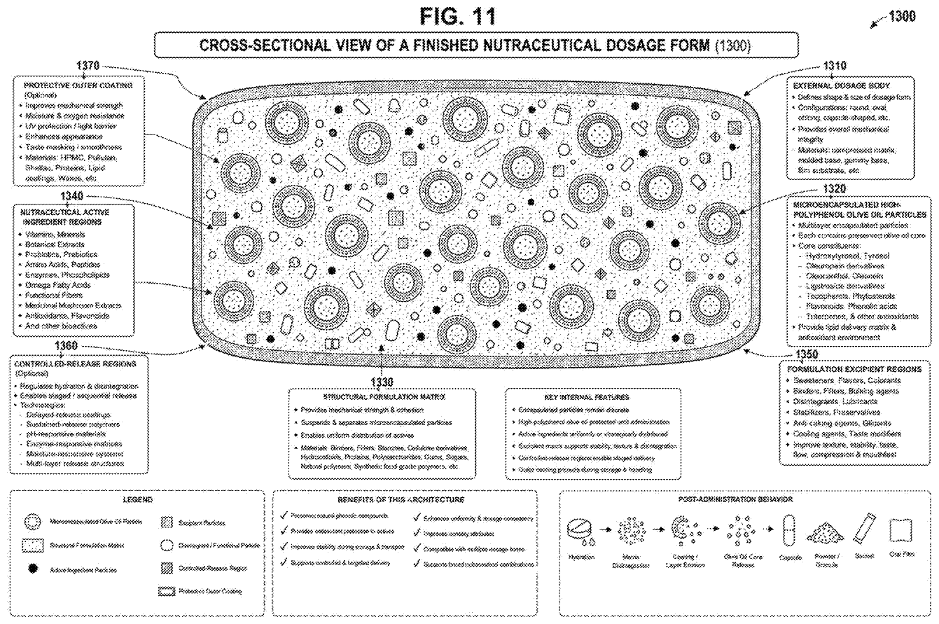

Referring now to FIG. 11, there is illustrated a representative cross-sectional view of a finished nutraceutical dosage form (1300) according to one embodiment of the present invention. The illustrated embodiment depicts the internal structural organization of an exemplary oral dosage form incorporating the high-polyphenol olive oil delivery platform. Although the figure is illustrated as a generally compressed tablet for purposes of explanation, the internal architecture described herein may likewise be incorporated into lozenges, orally disintegrating tablets, chewable tablets, gummies, oral films, capsules, powders, granules, nutritional confectionery products, sachets, or other orally administrable nutraceutical formulations.

As illustrated, the finished dosage form (1300) generally comprises an external dosage body (1310) enclosing a plurality of microencapsulated high-polyphenol olive oil particles (1320) distributed throughout an internal structural formulation matrix (1330). The dosage form further includes one or more nutraceutical active ingredient regions (1340), formulation excipient regions (1350), optional controlled-release regions (1360), and an optional protective outer coating (1370). Collectively, these structural components establish a consumer-ready nutraceutical product configured to preserve naturally occurring phenolic compounds while delivering one or more nutraceutical active ingredients.

The external dosage body (1310) defines the overall geometry of the finished nutraceutical composition. Depending upon the intended product, the dosage body may exhibit substantially circular, oval, oblong, cylindrical, polygonal, spherical, hemispherical, cuboidal, irregular, or customized configurations. The invention is not limited to any particular external geometry, and the dimensions may vary according to dosage strength, intended consumer population, manufacturing technology, and desired administration characteristics.

Distributed throughout the dosage body are a plurality of microencapsulated high-polyphenol olive oil particles (1320). Each encapsulated particle may possess the multilayer architecture previously described with reference to FIG. 7, including a high-polyphenol olive oil core surrounded by one or more protective encapsulating layers. In preferred embodiments, the encapsulated particles remain substantially intact during manufacturing while preserving the naturally occurring concentrations of hydroxytyrosol, tyrosol, oleuropein derivatives, oleocanthal, oleacein, ligstroside derivatives, tocopherols, phytosterols, flavonoids, phenolic acids, triterpenes, and additional naturally occurring antioxidant compounds.

In various embodiments, the encapsulated particles (1320) are substantially uniformly dispersed throughout the dosage form to establish a homogeneous high-polyphenol olive oil delivery matrix. Alternatively, the particles may be distributed in one or more discrete regions, multiple layers, concentric zones, longitudinal gradients, radial gradients, compartmentalized sections, or other spatial arrangements depending upon the desired release characteristics and manufacturing process.

Surrounding the encapsulated particles is the structural formulation matrix (1330). The structural matrix provides mechanical integrity to the dosage form while maintaining separation among adjacent encapsulated particles. Representative structural matrix materials include binders, fillers, bulking agents, compression aids, hydrocolloids, cellulose derivatives, starches, modified starches, proteins, polysaccharides, gums, sugars, sugar alcohols, natural polymers, synthetic food-grade polymers, gelatin, pectin, alginate, carrageenan, or combinations thereof.

In preferred embodiments, the structural formulation matrix (1330) is selected to provide adequate mechanical strength while minimizing undesirable interactions with the encapsulated olive oil particles. The matrix may additionally contribute to disintegration characteristics, moisture regulation, compression performance, mouthfeel, and overall consumer acceptability.

The dosage form further comprises one or more nutraceutical active ingredient regions (1340). Representative active ingredients include vitamins, minerals, botanical extracts, probiotics, prebiotics, amino acids, peptides, enzymes, phospholipids, omega fatty acids, functional fibers, medicinal mushroom extracts, flavonoids, carotenoids, antioxidants, proteins, fermentation products, marine-derived ingredients, trace minerals, adaptogens, or combinations thereof. These active ingredients may be incorporated as discrete particles, coated particles, granules, encapsulated materials, dissolved phases, or substantially homogeneous dispersions throughout the structural formulation matrix.

In certain embodiments, selected nutraceutical ingredients may associate directly with the encapsulated olive oil particles. Lipophilic compounds may partition into or interact with the lipid environment established by the high-polyphenol olive oil, whereas hydrophilic compounds remain dispersed within the surrounding structural matrix. Such interactions may facilitate improved compatibility among chemically diverse nutraceutical ingredients.

The dosage form further includes one or more formulation excipient regions (1350). Representative excipients include sweeteners, flavoring agents, coloring agents, lubricants, glidants, anti-caking agents, stabilizers, preservatives, cooling agents, taste modifiers, pH-adjusting agents, saliva-stimulating agents, disintegrants, compression lubricants, processing aids, coating materials, and additional excipients commonly utilized in nutraceutical formulations.

In embodiments configured as lozenges, chewable tablets, gummies, or orally disintegrating tablets, the formulation excipients (1350) may additionally improve sensory properties by reducing the perceived bitterness, pungency, peppery sensation, or throat irritation naturally associated with high-polyphenol olive oils. Such improvement may be achieved through flavor masking, sweetness enhancement, aroma modulation, controlled release, or combinations thereof without substantially reducing the naturally occurring phenolic composition of the encapsulated olive oil.

In certain embodiments, the dosage form further comprises one or more controlled-release regions (1360). The controlled-release regions regulate hydration, disintegration, diffusion, dissolution, or erosion of selected portions of the dosage form following administration. Representative controlled-release technologies include multilayer tablets, gradient-density matrices, delayed-release coatings, sustained-release polymers, moisture-responsive matrices, enzyme-responsive materials, pH-sensitive materials, lipid-controlled diffusion systems, hydrogel-forming materials, or combinations thereof.

The controlled-release regions (1360) may cooperate with the encapsulated olive oil particles to provide sequential release of nutraceutical ingredients and the high-polyphenol olive oil. In certain embodiments, water-soluble ingredients are released initially, followed by gradual release of the encapsulated olive oil as the protective encapsulating layers progressively dissolve or degrade under physiological conditions.

The dosage form may additionally comprise an optional protective outer coating (1370) surrounding the external dosage body. The protective coating may improve mechanical durability, moisture resistance, oxygen resistance, ultraviolet protection, appearance, swallowability, taste masking, gloss, or consumer acceptability. Representative coating materials include hydroxypropyl methylcellulose, pullulan, shellac, cellulose derivatives, proteins, polysaccharides, lipid coatings, waxes, enteric coatings, moisture-barrier coatings, edible polymers, or combinations thereof.

During manufacture, the encapsulated particles (1320) become substantially embedded within the structural formulation matrix (1330) while retaining their individual structural integrity. Consequently, each encapsulated particle continues to function as an independent microreservoir containing preserved high-polyphenol olive oil, thereby reducing premature interaction between the olive oil and surrounding formulation components during storage.

Upon consumer administration, saliva, gastric fluids, intestinal fluids, digestive enzymes, or physiological pH conditions gradually penetrate the dosage body (1310). Hydration initially softens or disintegrates the structural formulation matrix (1330), thereby exposing individual encapsulated particles (1320). Depending upon the selected encapsulation architecture, the encapsulated particles subsequently undergo immediate, delayed, sustained, staged, pH-responsive, moisture-responsive, enzyme-responsive, or sequential release to liberate the preserved high-polyphenol olive oil together with associated nutraceutical ingredients.

One significant feature illustrated by FIG. 11 is that the encapsulated particles remain structurally discrete within the finished dosage form rather than merging into a continuous oil phase. This discrete particle architecture minimizes oil migration, phase separation, oxidation, ingredient incompatibility, and undesirable changes in texture while improving storage stability and dosage uniformity.

Another important aspect illustrated by FIG. 11 is the multifunctional role of the encapsulated olive oil particles within the dosage form. In addition to functioning as biologically active nutraceutical ingredients, the particles establish localized antioxidant-rich microenvironments capable of supporting preservation of oxidation-sensitive nutraceutical ingredients distributed throughout the formulation.

In various embodiments, the number, size, concentration, distribution pattern, encapsulation architecture, release characteristics, and biochemical composition of the encapsulated particles may be modified according to the intended commercial application without departing from the principles of the present invention. Likewise, the structural formulation matrix, active ingredient composition, excipient selection, coating materials, and dosage-form geometry may be varied to accommodate different manufacturing technologies and nutritional applications.

Accordingly, FIG. 11 illustrates a representative finished nutraceutical dosage form in which the external dosage body (1310), microencapsulated high-polyphenol olive oil particles (1320), structural formulation matrix (1330), nutraceutical active ingredient regions (1340), formulation excipient regions (1350), optional controlled-release regions (1360), and optional protective outer coating (1370) collectively establish a stable oral dosage form capable of preserving naturally occurring olive oil phenolic compounds while providing controlled delivery of both the high-polyphenol olive oil and one or more co-formulated nutraceutical ingredients.

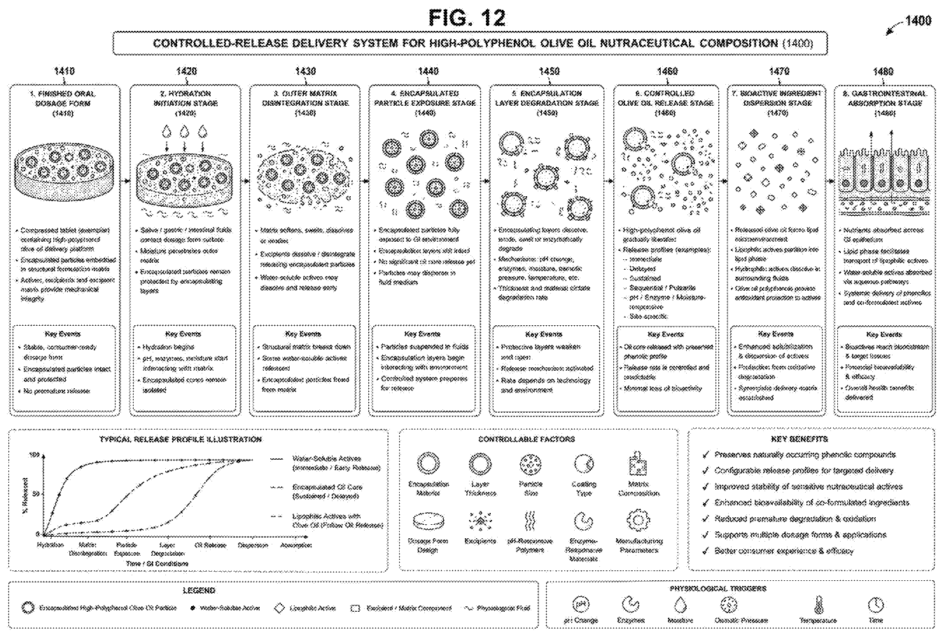

Referring now to FIG. 12, there is illustrated an exemplary controlled-release delivery system (1400) for a high-polyphenol olive oil nutraceutical composition according to one embodiment of the present invention. The illustrated embodiment demonstrates the progressive release of encapsulated high-polyphenol olive oil and one or more co-formulated nutraceutical ingredients following oral administration. Unlike conventional nutraceutical formulations that release active ingredients immediately upon exposure to physiological fluids, the present invention provides a configurable delivery platform capable of producing immediate, delayed, sustained, sequential, staged, site-specific, or physiologically responsive release profiles while substantially preserving the naturally occurring phenolic composition of the olive oil until the desired stage of administration.

As illustrated, the controlled-release delivery system (1400) generally comprises a finished oral dosage form (1410), a hydration initiation stage (1420), an outer matrix disintegration stage (1430), an encapsulated particle exposure stage (1440), an encapsulation layer degradation stage (1450), a controlled olive oil release stage (1460), a bioactive ingredient dispersion stage (1470), and a gastrointestinal absorption stage (1480). Although the illustrated embodiment depicts one representative release sequence, additional release mechanisms and physiological pathways may be employed without departing from the principles of the present invention.

The process begins with the finished oral dosage form (1410). The dosage form may comprise a lozenge, fast-dissolving tablet, orally disintegrating tablet, chewable tablet, gummy, capsule, powder, granule, oral film, sachet, nutritional beverage, nutritional shot, functional confectionery product, or another orally administrable nutraceutical formulation incorporating the high-polyphenol olive oil delivery platform. The dosage form contains a plurality of encapsulated particles distributed throughout a structural formulation matrix as previously described with reference to FIG. 11.

Following administration by a consumer, the dosage form enters the hydration initiation stage (1420). During this stage, saliva, gastric fluids, intestinal fluids, water, or other physiological fluids contact the outer surfaces of the dosage form. Hydration gradually penetrates the structural formulation matrix while the encapsulated high-polyphenol olive oil particles remain substantially protected by their surrounding encapsulating layers.

The hydration process may occur within the oral cavity, stomach, small intestine, or another physiological location depending upon the selected dosage form and release characteristics. Representative hydration conditions include saliva-mediated dissolution, gastric fluid exposure, intestinal fluid exposure, pH variation, digestive enzyme activity, moisture diffusion, osmotic transport, or combinations thereof.

Continued hydration results in the outer matrix disintegration stage (1430). During this stage, the structural formulation matrix surrounding the encapsulated particles gradually softens, dissolves, erodes, disintegrates, swells, disperses, or otherwise loses structural integrity. Representative mechanisms include dissolution of water-soluble excipients, hydrogel swelling, polymer erosion, enzymatic degradation, mechanical fragmentation, or combinations thereof.

In preferred embodiments, the rate of matrix disintegration is controlled independently of the encapsulated olive oil particles. Consequently, the structural dosage form may release selected water-soluble nutraceutical ingredients before significant release of the encapsulated olive oil occurs, thereby establishing sequential delivery of different formulation components.

Following matrix disintegration, the encapsulated particles enter the encapsulated particle exposure stage (1440). Individual encapsulated particles become exposed to the surrounding physiological environment while maintaining substantially intact encapsulating layers. Exposure of the particles does not necessarily result in immediate release of the olive oil core, thereby permitting additional control over the release profile.

The encapsulated particles subsequently undergo the encapsulation layer degradation stage (1450). Depending upon the selected encapsulation architecture, one or more encapsulating layers gradually dissolve, erode, swell, rupture, diffuse, enzymatically degrade, hydrolyze, or otherwise permit controlled access to the enclosed high-polyphenol olive oil. In various embodiments, degradation may occur in response to physiological pH, digestive enzymes, moisture content, ionic strength, temperature, microbial activity, osmotic pressure, mechanical compression, or combinations thereof.

Representative encapsulation materials may be selected to provide different release characteristics. Water-soluble encapsulating materials may facilitate rapid release, whereas moisture-resistant or enzyme-degradable encapsulating materials may provide delayed or sustained release. Likewise, enteric materials may postpone release until exposure to intestinal pH conditions, thereby protecting the encapsulated olive oil from premature gastric release.

Following degradation of the encapsulating layers, the system proceeds to the controlled olive oil release stage (1460). During this stage, the high-polyphenol olive oil is gradually liberated from the encapsulated particles while substantially maintaining the naturally occurring concentrations of hydroxytyrosol, tyrosol, oleuropein derivatives, oleocanthal, oleacein, ligstroside derivatives, tocopherols, phytosterols, flavonoids, phenolic acids, triterpenes, and additional naturally occurring antioxidant constituents.

The release characteristics of the olive oil may be configured according to the desired nutritional application. Representative release profiles include:
(1461) Immediate release;
(1462) Delayed release;
(1463) Sustained release;
(1464) Sequential release;
(1465) Pulsatile release;
(1466) Site-specific release;
(1467) pH-responsive release;
(1468) Enzyme-responsive release;
(1469) Moisture-responsive release; and
(1470) Multi-phase release.

These release mechanisms may be achieved individually or in combination by appropriate selection of encapsulation materials, particle architecture, formulation composition, and dosage-form design.

The released olive oil subsequently establishes the bioactive ingredient dispersion stage (1470). During this stage, the liberated lipid phase promotes dispersion and transport of co-formulated lipophilic nutraceutical ingredients while simultaneously exposing hydrophilic ingredients to the surrounding physiological fluids. Naturally occurring phospholipids, monounsaturated fatty acids, and other lipid constituents may cooperate with the olive oil to establish localized lipid microenvironments capable of facilitating transport of lipid-compatible nutraceutical compounds.

In addition to functioning as a transport medium, the released high-polyphenol olive oil provides a naturally occurring antioxidant environment surrounding co-formulated nutraceutical ingredients. The preserved phenolic compounds may assist in reducing oxidative degradation of oxidation-sensitive vitamins, botanical extracts, omega fatty acids, carotenoids, flavonoids, and additional nutraceutical ingredients during the release process.

Following release and dispersion, the formulation proceeds to the gastrointestinal absorption stage (1480). During this stage, the released olive oil and associated nutraceutical ingredients become available for physiological absorption through the gastrointestinal tract. Lipophilic ingredients may partition into the lipid phase established by the released olive oil, whereas hydrophilic ingredients dissolve within surrounding physiological fluids before absorption. The invention does not require any particular absorption mechanism and encompasses normal physiological uptake pathways associated with orally administered nutraceutical compositions.

One significant feature illustrated by FIG. 12 is the ability to independently control release of the dosage-form matrix, encapsulated particles, and olive oil core. By separating these release events into distinct stages, the present invention provides greater flexibility in designing nutraceutical products exhibiting customized delivery characteristics while preserving naturally occurring phenolic compounds until the desired stage of administration.

Another important aspect illustrated by FIG. 12 is the preservation of the naturally occurring biochemical profile of the high-polyphenol olive oil throughout the release process. Unlike conventional formulations in which oxidation-sensitive constituents may degrade prior to administration, the present invention substantially maintains the phenolic composition until release of the olive oil from the encapsulated particles, thereby preserving the functional characteristics associated with premium high-polyphenol olive oils.

In certain embodiments, one or more stages of the controlled-release system may be modified through selection of alternative encapsulation materials, multilayer encapsulation architectures, particle dimensions, coating thicknesses, excipient compositions, dosage-form geometries, physiological response materials, or manufacturing parameters. Consequently, the release profile may be tailored for different consumer populations, dosage forms, nutritional applications, storage conditions, or commercial objectives without departing from the principles of the invention.

Furthermore, the controlled-release delivery system may be integrated with smart delivery technologies including sensor-responsive materials, programmable biodegradable coatings, microstructured encapsulation architectures, multilayer diffusion barriers, or advanced biomaterial systems configured to further regulate release kinetics while preserving the naturally occurring phenolic composition of the encapsulated olive oil.

Accordingly, FIG. 12 illustrates a comprehensive controlled-release delivery architecture in which the finished oral dosage form (1410), hydration initiation stage (1420), outer matrix disintegration stage (1430), encapsulated particle exposure stage (1440), encapsulation layer degradation stage (1450), controlled olive oil release stage (1460), bioactive ingredient dispersion stage (1470), and gastrointestinal absorption stage (1480) collectively establish a configurable delivery platform capable of preserving naturally occurring phenolic compounds while providing controlled and physiologically responsive delivery of high-polyphenol olive oil and one or more co-formulated nutraceutical ingredients.

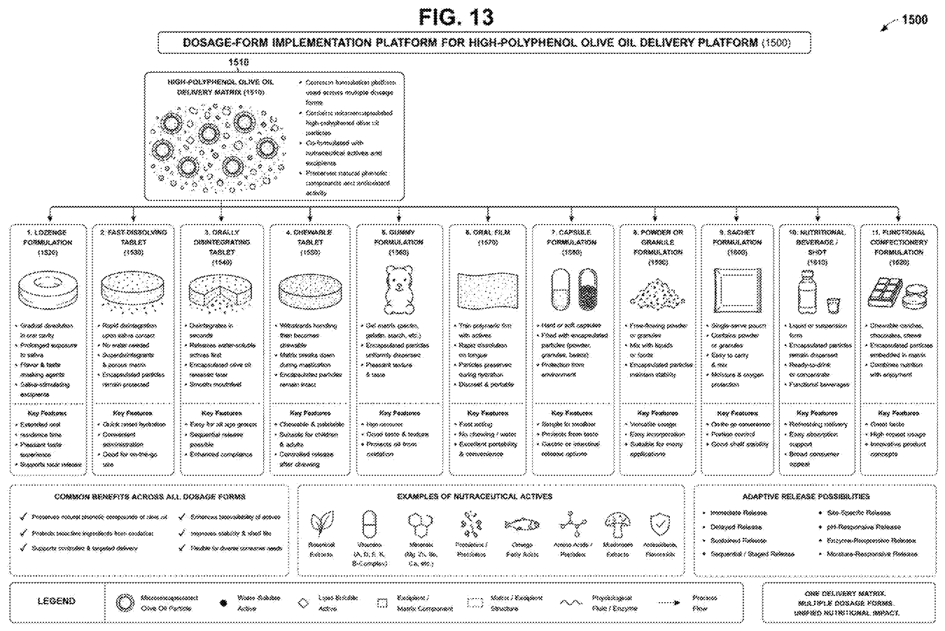

Referring now to FIG. 13, there is illustrated an exemplary dosage-form implementation platform (1500) according to one embodiment of the present invention. The illustrated embodiment demonstrates that the high-polyphenol olive oil delivery platform may be incorporated into numerous oral dosage forms while substantially preserving the underlying encapsulated particle architecture, naturally occurring phenolic composition, and multifunctional lipid delivery characteristics. Unlike conventional olive oil nutraceutical products that are generally restricted to liquid oils or softgel capsules, the present invention provides a dosage-form-independent platform adaptable to a wide variety of consumer-friendly formulations.

As illustrated, the dosage-form implementation platform (1500) generally comprises a high-polyphenol olive oil delivery matrix (1510) serving as a common formulation platform from which multiple finished dosage forms are produced. The delivery matrix (1510) contains a plurality of microencapsulated high-polyphenol olive oil particles together with one or more nutraceutical active ingredients and formulation excipients configured according to the desired commercial product.

In preferred embodiments, the underlying delivery matrix (1510) remains substantially unchanged while only the surrounding dosage-form architecture is modified. Consequently, substantially the same encapsulated high-polyphenol olive oil particles may be incorporated into numerous commercial products without altering the fundamental preservation and delivery principles of the invention.

A first representative dosage form comprises a lozenge formulation (1520). The lozenge may be configured for gradual dissolution within the oral cavity, thereby providing prolonged exposure of the formulation to saliva before swallowing. In various embodiments, flavor-modifying agents, sweeteners, cooling agents, saliva-stimulating agents, and taste-masking components may be incorporated to improve consumer acceptance while maintaining preservation of the naturally occurring phenolic compounds contained within the encapsulated olive oil particles.

A second representative dosage form comprises a fast-dissolving tablet (1530). The fast-dissolving tablet may be formulated to rapidly disintegrate upon contact with saliva without requiring water for administration. Representative formulation materials include rapidly dissolving excipients, superdisintegrants, porous matrices, rapidly hydrating polymers, or combinations thereof. The encapsulated olive oil particles remain substantially protected during the rapid disintegration process until exposure to predetermined physiological conditions.

A third representative dosage form comprises an orally disintegrating tablet (1540). The orally disintegrating tablet may gradually disperse within the oral cavity while releasing selected water-soluble ingredients before controlled release of the encapsulated olive oil. Such formulations may be particularly suitable for pediatric, geriatric, or convenience-oriented applications.

A fourth representative dosage form comprises a chewable tablet (1550). The chewable tablet may possess sufficient mechanical strength for handling while remaining readily chewable by the consumer. Following mastication, the structural formulation matrix is disrupted, exposing the encapsulated olive oil particles while substantially preserving their protective encapsulating layers until further physiological release conditions are encountered.

A fifth representative dosage form comprises a gummy formulation (1560). In this embodiment, the encapsulated high-polyphenol olive oil particles are dispersed throughout a gelatin-based, pectin-based, starch-based, carrageenan-based, agar-based, alginate-based, or other edible gel matrix. The encapsulated particles remain substantially isolated from the surrounding gummy matrix, thereby reducing oil migration, oxidation, and undesirable texture changes while maintaining desirable consumer sensory characteristics.

A sixth representative dosage form comprises an oral film (1570). The oral film may comprise one or more thin edible polymer layers containing dispersed encapsulated olive oil particles. Representative film-forming materials include pullulan, hydroxypropyl methylcellulose, polyvinyl alcohol, starch derivatives, alginates, pectins, cellulose derivatives, proteins, or combinations thereof. The oral film may dissolve rapidly upon placement within the oral cavity while preserving the structural integrity of the encapsulated particles during initial hydration.

A seventh representative dosage form comprises a capsule formulation (1580). The capsule may contain encapsulated high-polyphenol olive oil particles in powder, granule, bead, pellet, multiparticulate, or related particulate forms. Hard-shell capsules, soft capsules, plant-based capsules, gelatin capsules, hydroxypropyl methylcellulose capsules, pullulan capsules, or additional capsule systems may be utilized according to the intended application.

An eighth representative dosage form comprises a powder or granule formulation (1590). The encapsulated particles may be blended with one or more nutraceutical ingredients to produce free-flowing powders or granules suitable for direct oral administration, suspension within liquids, incorporation into beverages, blending with foods, filling into sachets, or further processing into additional dosage forms.

A ninth representative dosage form comprises a sachet formulation (1600). The sachet may contain powders, granules, multiparticulates, beads, or additional particulate materials incorporating the encapsulated olive oil delivery matrix. The sachet contents may subsequently be dispersed within water, juice, milk, yogurt, smoothies, nutritional beverages, or other ingestible liquids prior to administration.

A tenth representative dosage form comprises a nutritional beverage or nutritional shot formulation (1610). In such embodiments, the encapsulated particles remain suspended or dispersed within a liquid carrier while maintaining protection of the naturally occurring phenolic compounds. Representative beverage formulations include ready-to-drink beverages, concentrates, reconstitutable powders, emulsions, suspensions, liquid nutritional supplements, meal-replacement beverages, sports nutrition beverages, wellness shots, and related nutritional products.

An eleventh representative dosage form comprises a functional confectionery formulation (1620). Representative confectionery products include chewable confections, compressed candies, soft candies, hard candies, nutritional chews, fruit chews, nutritional gummies, chocolate-based products, coated confectionery products, or additional edible consumer products incorporating the encapsulated olive oil delivery matrix.

Although FIG. 13 illustrates representative dosage forms, the invention is not limited to the specifically illustrated embodiments. The delivery platform may likewise be incorporated into nutritional bars, protein bars, meal supplements, chewable gels, edible strips, compressed multiparticulates, orally dispersible granules, multiparticulate capsules, bead-filled sachets, layered tablets, multilayer gummies, layered confectionery products, frozen nutritional products, refrigerated nutritional formulations, fermented nutritional products, or additional edible dosage forms without departing from the principles of the invention.

One significant feature illustrated by FIG. 13 is that each dosage form incorporates substantially the same high-polyphenol olive oil delivery matrix (1510). Rather than requiring independent formulation strategies for each commercial product, the encapsulated olive oil particles establish a common technological platform adaptable across numerous dosage forms while preserving the naturally occurring phenolic composition and antioxidant characteristics of the selected olive oil.

Another important aspect illustrated by FIG. 13 is the manufacturing flexibility provided by the present invention. Manufacturers may produce multiple commercial products utilizing substantially identical encapsulated olive oil particles while varying only the surrounding dosage-form architecture, excipient composition, flavor system, sweetener system, release characteristics, or physical presentation according to consumer preferences and intended market applications.

In certain embodiments, different dosage forms may incorporate distinct release profiles. For example, a lozenge may provide gradual oral release, a fast-dissolving tablet may provide immediate hydration followed by delayed olive oil release, a capsule may provide gastric or intestinal release, and a gummy formulation may provide controlled release following mastication. Accordingly, the same underlying delivery platform may be configured for different physiological delivery profiles without altering the encapsulated high-polyphenol olive oil itself.

The dosage-form implementation platform may further be adapted for specialized nutritional applications including cardiovascular health formulations, cognitive support formulations, immune-support products, sports nutrition products, healthy aging formulations, metabolic wellness supplements, gastrointestinal health products, pediatric nutritional formulations, geriatric nutritional formulations, prenatal supplements, veterinary formulations, medical nutrition products, or personalized nutrition systems while preserving the structural and biochemical characteristics of the underlying delivery matrix.

In various embodiments, the dosage forms may additionally incorporate protective coatings, multilayer architectures, compartmentalized structures, flavor-modulating layers, moisture barriers, oxygen barriers, enteric coatings, identification markings, edible printing, dosage indicators, tamper-evident structures, or additional formulation features according to the intended commercial application.

Accordingly, FIG. 13 illustrates a comprehensive dosage-form implementation platform in which the high-polyphenol olive oil delivery matrix (1510) serves as a common formulation architecture for the lozenge formulation (1520), fast-dissolving tablet (1530), orally disintegrating tablet (1540), chewable tablet (1550), gummy formulation (1560), oral film (1570), capsule formulation (1580), powder or granule formulation (1590), sachet formulation (1600), nutritional beverage or nutritional shot formulation (1610), and functional confectionery formulation (1620). Collectively, these embodiments demonstrate the dosage-form-independent nature of the present invention, enabling preservation and delivery of high-polyphenol olive oil across a diverse range of nutraceutical products while maintaining the structural, biochemical, and functional characteristics of the encapsulated delivery platform.

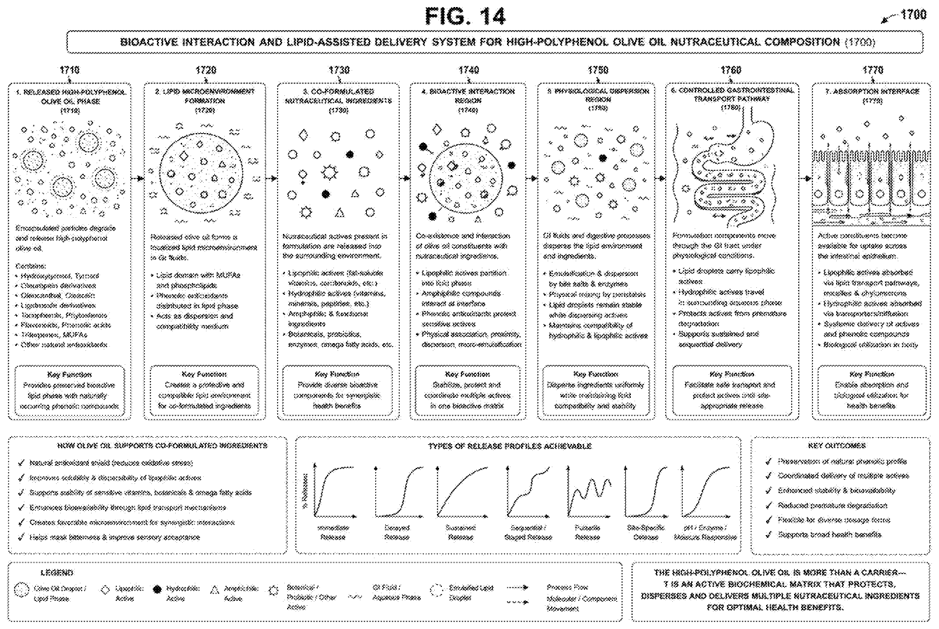

Referring now to FIG. 14, there is illustrated an exemplary bioactive interaction and lipid-assisted delivery system (1700) according to one embodiment of the present invention. The illustrated embodiment demonstrates the functional interactions occurring after controlled release of the encapsulated high-polyphenol olive oil from the dosage form and illustrates how the released olive oil cooperates with one or more co-formulated nutraceutical ingredients to establish a multifunctional lipid-assisted delivery environment. Unlike conventional nutraceutical formulations in which active ingredients are independently released into physiological fluids, the present invention utilizes the released high-polyphenol olive oil as an active biochemical matrix that interacts with, stabilizes, disperses, and facilitates delivery of multiple nutraceutical components.

As illustrated, the bioactive interaction and lipid-assisted delivery system (1700) generally comprises a released high-polyphenol olive oil phase (1710), a lipid microenvironment (1720), one or more co-formulated nutraceutical ingredients (1730), a bioactive interaction region (1740), a physiological dispersion region (1750), a controlled gastrointestinal transport pathway (1760), and an absorption interface (1770). Collectively, these elements illustrate the functional behavior of the delivery platform following release from the encapsulated particles.

Following degradation of the encapsulating layers as previously described with reference to FIG. 12, the encapsulated particles release the high-polyphenol olive oil phase (1710). The released olive oil contains substantially preserved naturally occurring hydroxytyrosol, tyrosol, oleuropein derivatives, oleocanthal, oleacein, ligstroside derivatives, tocopherols, phytosterols, flavonoids, phenolic acids, triterpenes, monounsaturated fatty acids, and additional naturally occurring constituents selected during the quality characterization process.

Rather than functioning solely as a nutritional ingredient, the released olive oil establishes a lipid microenvironment (1720) surrounding the released formulation components. The lipid microenvironment represents a localized dispersion phase containing naturally occurring olive oil lipids together with preserved phenolic constituents capable of interacting with surrounding nutraceutical ingredients.

In preferred embodiments, the lipid microenvironment (1720) remains substantially homogeneous for a sufficient period to permit interaction between the released olive oil and one or more co-formulated nutraceutical ingredients. The localized lipid phase may reduce immediate phase separation while promoting uniform dispersion of ingredients exhibiting differing chemical properties.

The lipid microenvironment subsequently surrounds one or more co-formulated nutraceutical ingredients (1730). Representative nutraceutical ingredients include vitamins, minerals, botanical extracts, medicinal plant derivatives, probiotics, prebiotics, amino acids, peptides, enzymes, phospholipids, omega fatty acids, carotenoids, flavonoids, proteins, medicinal mushroom extracts, fermentation products, marine-derived compounds, trace elements, functional fibers, adaptogens, and combinations thereof.

In various embodiments, lipophilic nutraceutical ingredients may partially partition into or associate with the lipid microenvironment established by the released olive oil, whereas hydrophilic ingredients remain dispersed within the surrounding aqueous physiological fluids. Amphiphilic compounds may interact with both phases simultaneously. Such interactions permit chemically diverse nutraceutical ingredients to coexist within the overall delivery platform.

The interaction between the released olive oil and the nutraceutical ingredients forms a bioactive interaction region (1740). Within this region, naturally occurring olive oil constituents coexist with the released nutraceutical ingredients while maintaining the biochemical integrity of the overall formulation. The invention does not require any specific molecular interaction or chemical reaction, and the bioactive interaction region may comprise physical association, lipid partitioning, dispersion, emulsification, suspension, proximity, or combinations thereof.

In certain embodiments, naturally occurring antioxidant compounds present within the released olive oil may contribute to maintenance of the local biochemical environment surrounding oxidation-sensitive nutraceutical ingredients during the release process. The preserved phenolic compounds, tocopherols, flavonoids, and additional naturally occurring antioxidant constituents may collectively reduce undesirable oxidative degradation that could otherwise occur following release from the dosage form.

The released formulation subsequently enters the physiological dispersion region (1750). During this stage, gastrointestinal fluids, digestive secretions, physiological mixing, peristaltic motion, and normal digestive processes gradually disperse the released olive oil and associated nutraceutical ingredients throughout the surrounding physiological environment.

In preferred embodiments, the released olive oil establishes a lipid dispersion phase capable of maintaining compatibility among multiple formulation ingredients exhibiting different solubility characteristics. Such compatibility enables simultaneous administration of hydrophilic, amphiphilic, and lipophilic nutraceutical compounds within a single dosage form while reducing undesirable phase separation immediately following release.

Following physiological dispersion, the released formulation proceeds along the controlled gastrointestinal transport pathway (1760). The transport pathway represents the movement of the released olive oil and associated nutraceutical ingredients through the gastrointestinal tract under normal physiological conditions. The invention does not require any particular transport mechanism and encompasses normal digestive transport associated with orally administered nutraceutical formulations.

During gastrointestinal transport, naturally occurring monounsaturated fatty acids, phospholipids, and additional lipid constituents present within the released olive oil may cooperate to establish localized lipid domains capable of supporting dispersion of lipophilic nutraceutical compounds. Likewise, water-soluble ingredients remain dispersed within surrounding aqueous physiological fluids, thereby permitting simultaneous transport of chemically distinct ingredients.

The transported formulation ultimately reaches the absorption interface (1770). The absorption interface schematically represents the physiological region at which released formulation components become available for uptake through normal biological absorption mechanisms. The invention is not limited to any specific absorption pathway, transport protein, metabolic mechanism, or biological target, and the illustrated absorption interface merely represents availability of the released nutraceutical ingredients for normal physiological processing following oral administration.

One significant feature illustrated by FIG. 14 is that the high-polyphenol olive oil functions as substantially more than a passive carrier oil. Upon release, the olive oil establishes an active lipid microenvironment that supports dispersion, compatibility, and coordinated delivery of numerous nutraceutical ingredients while simultaneously preserving the naturally occurring biochemical profile selected during manufacturing.

Another important aspect illustrated by FIG. 14 is the multifunctional role of the preserved naturally occurring phenolic compounds. Rather than being isolated active ingredients, the phenolic constituents remain distributed throughout the released lipid phase where they continue to form part of the overall biochemical environment associated with the delivery platform.

In certain embodiments, the interaction between the released olive oil and co-formulated nutraceutical ingredients may be modified according to the intended formulation. The relative concentrations of olive oil, encapsulated particles, nutraceutical ingredients, lipid-compatible compounds, hydrophilic compounds, amphiphilic compounds, excipients, and release-controlling materials may be adjusted without departing from the principles of the invention.

Furthermore, the lipid-assisted delivery system may be utilized with formulations configured for cardiovascular wellness, healthy aging, metabolic support, cognitive support, immune support, gastrointestinal wellness, sports nutrition, general nutritional supplementation, personalized nutrition, medical nutrition, veterinary nutrition, or additional nutraceutical applications while preserving the underlying delivery platform architecture.

Although FIG. 14 illustrates one representative interaction sequence, additional interaction mechanisms including multilayer lipid dispersion systems, multiparticulate release systems, sequential ingredient release, compartmentalized formulations, self-emulsifying systems, self-microemulsifying systems, hybrid encapsulation technologies, or additional lipid-assisted delivery mechanisms may likewise be incorporated within the scope of the present invention.

Accordingly, FIG. 14 illustrates a representative bioactive interaction and lipid-assisted delivery architecture in which the released high-polyphenol olive oil phase (1710) establishes a localized lipid microenvironment (1720) surrounding one or more co-formulated nutraceutical ingredients (1730) to form a bioactive interaction region (1740). The released formulation subsequently undergoes physiological dispersion (1750), progresses through the controlled gastrointestinal transport pathway (1760), and reaches the absorption interface (1770). Collectively, these components demonstrate how the present invention utilizes preserved high-polyphenol olive oil not merely as a nutraceutical ingredient but as a multifunctional lipid delivery matrix that supports coordinated dispersion and delivery of diverse nutraceutical components following oral administration.

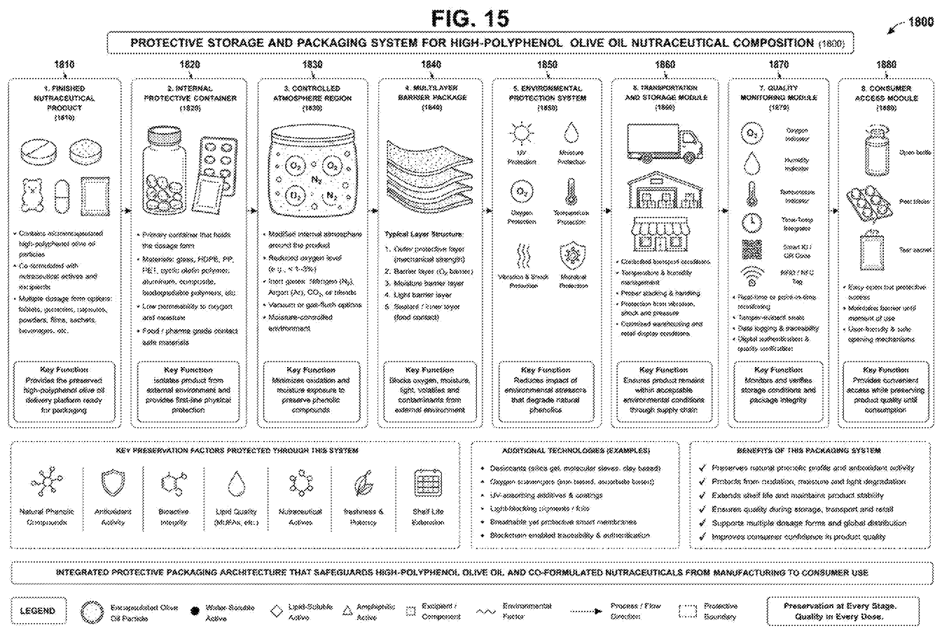

Referring now to FIG. 15, there is illustrated an exemplary protective storage and packaging system (1800) configured to preserve the biochemical integrity of high-polyphenol olive oil-containing nutraceutical compositions according to one embodiment of the present invention. The illustrated embodiment demonstrates a comprehensive storage architecture that minimizes degradation of naturally occurring phenolic compounds throughout commercial packaging, warehousing, transportation, retail display, consumer storage, and product administration. Unlike conventional nutraceutical packaging that primarily functions as a physical container, the present invention provides a preservation-oriented packaging platform specifically configured to maintain the naturally occurring biochemical profile of high-polyphenol olive oil during the post-manufacturing lifecycle.

As illustrated, the protective storage and packaging system (1800) generally comprises a finished nutraceutical product (1810), an internal protective container (1820), a controlled atmosphere region (1830), a multilayer barrier package (1840), an environmental protection system (1850), a transportation and storage module (1860), a quality monitoring module (1870), and a consumer access module (1880). Collectively, these components establish an integrated preservation architecture configured to substantially maintain the naturally occurring phenolic composition of the encapsulated olive oil until product administration.

The system begins with the finished nutraceutical product (1810) produced according to the manufacturing methodology previously described with reference to FIG. 10. The finished product may comprise lozenges, chewable tablets, fast-dissolving tablets, orally disintegrating tablets, gummies, capsules, oral films, powders, granules, sachets, beverages, nutritional shots, functional confectionery products, multiparticulate formulations, or other dosage forms incorporating the encapsulated high-polyphenol olive oil delivery platform.

The finished nutraceutical product is positioned within an internal protective container (1820). Depending upon the commercial product configuration, the internal container may comprise a bottle, blister cavity, capsule shell, sachet compartment, pouch, cartridge, vial, tube, tray, compartmentalized package, or other food-grade or pharmaceutical-grade containment structure. Representative container materials include glass, stainless steel, aluminum, multilayer polymer laminates, polyethylene, polypropylene, polyethylene terephthalate, cyclic olefin polymers, composite materials, biodegradable packaging materials, plant-derived polymers, or combinations thereof.

In preferred embodiments, the internal protective container (1820) is selected to minimize migration of oxygen, moisture, volatile compounds, ultraviolet radiation, and environmental contaminants toward the encapsulated high-polyphenol olive oil. The container may additionally exhibit reduced permeability to atmospheric gases and water vapor while maintaining compatibility with long-term commercial storage.

Surrounding the packaged dosage form is a controlled atmosphere region (1830). The controlled atmosphere region may comprise an enclosed internal environment containing reduced oxygen concentrations, inert gases, controlled humidity, reduced moisture content, modified atmospheric composition, vacuum conditions, or combinations thereof. Representative inert gases include nitrogen, argon, carbon dioxide, helium, or mixtures thereof. The controlled atmosphere assists in reducing oxidation of naturally occurring phenolic compounds during storage.

In certain embodiments, oxygen concentrations within the controlled atmosphere region (1830) are maintained below ambient atmospheric concentrations through inert-gas flushing, vacuum packaging, oxygen displacement, oxygen scavenging materials, or combinations thereof. Likewise, moisture concentrations may be reduced through desiccants, moisture-absorbing materials, humidity-control systems, or barrier packaging technologies.

Surrounding the controlled atmosphere region is a multilayer barrier package (1840). The multilayer barrier package comprises one or more protective layers configured to reduce transmission of oxygen, water vapor, ultraviolet radiation, visible light, volatile compounds, environmental contaminants, or mechanical stress. Representative barrier structures include multilayer laminates, foil laminates, metallized polymer films, ultraviolet-resistant coatings, moisture-barrier coatings, oxygen-barrier polymers, light-blocking pigments, ceramic barrier coatings, nanocomposite barrier films, cellulose-based barrier materials, biodegradable multilayer packaging systems, or combinations thereof.

In preferred embodiments, the multilayer barrier package (1840) comprises a plurality of functional layers, each configured to provide a different protective function. For example, an inner layer may provide food-contact compatibility, an intermediate layer may reduce oxygen permeability, an additional layer may reduce moisture transmission, while an outer layer may provide mechanical durability and ultraviolet protection.

The packaging system further comprises an environmental protection system (1850). The environmental protection system is configured to reduce exposure of the packaged nutraceutical product to environmental conditions capable of degrading naturally occurring phenolic compounds. Representative environmental factors include elevated temperatures, temperature cycling, humidity fluctuations, oxygen exposure, ultraviolet radiation, visible light, ozone, mechanical vibration, physical shock, compression, atmospheric pollutants, microbial contamination, and prolonged storage.

In certain embodiments, the environmental protection system (1850) incorporates temperature-control materials, thermal insulation, ultraviolet-absorbing coatings, oxygen scavengers, desiccants, humidity regulators, antimicrobial materials, shock-absorbing structures, vibration-dampening components, light-reflective coatings, or combinations thereof. Such preservation technologies collectively assist in maintaining the biochemical profile of the encapsulated high-polyphenol olive oil throughout commercial distribution.

Following packaging, the product enters the transportation and storage module (1860). The transportation and storage module represents movement of the packaged product through manufacturing facilities, distribution centers, warehouses, retail environments, shipping networks, healthcare providers, pharmacies, or consumer storage environments. Throughout these operations, the protective packaging system continues to isolate the encapsulated olive oil from degradation-promoting environmental conditions.

In preferred embodiments, transportation and storage conditions may be monitored according to predetermined environmental specifications including temperature ranges, humidity limits, storage duration, transportation duration, vibration exposure, atmospheric composition, and package integrity. Such monitoring assists in maintaining product quality throughout the commercial supply chain.

The protective packaging system may additionally comprise a quality monitoring module (1870). Representative monitoring technologies include oxygen indicators, humidity indicators, freshness indicators, temperature indicators, time-temperature integrators, tamper-evident seals, pressure indicators, ultraviolet exposure indicators, radio-frequency identification devices, near-field communication devices, digital authentication systems, blockchain traceability systems, machine-readable identifiers, barcodes, QR codes, smart packaging electronics, environmental sensors, or combinations thereof.

In certain embodiments, the quality monitoring module continuously or periodically records environmental conditions encountered during storage and transportation. The resulting data may be utilized to verify maintenance of acceptable storage conditions or to identify environmental events capable of affecting preservation of the naturally occurring phenolic profile.

Upon consumer use, the packaged product enters the consumer access module (1880). Representative access mechanisms include removal of a bottle cap, opening of a blister package, tearing of a sachet, peeling of a foil seal, opening of a pouch, removal of an oral film, dispensing from a cartridge, or other consumer-accessible packaging mechanisms. The protective packaging remains substantially intact until the time of intended administration, thereby minimizing unnecessary environmental exposure before consumer use.

One significant feature illustrated by FIG. 15 is that preservation of naturally occurring phenolic compounds continues beyond manufacturing and extends throughout the commercial lifecycle of the product. The coordinated interaction among the internal container, controlled atmosphere, multilayer barrier package, environmental protection technologies, transportation controls, and quality monitoring systems substantially reduces cumulative degradation that might otherwise occur during storage and distribution.

Another important aspect illustrated by FIG. 15 is the modular nature of the packaging architecture. Individual protective technologies may be incorporated, omitted, substituted, or combined according to the intended dosage form, expected shelf life, climatic conditions, transportation requirements, regulatory standards, commercial distribution channels, or packaging costs while maintaining the underlying preservation objectives of the invention.

In certain embodiments, the packaging architecture may additionally be integrated with automated warehouse management systems, cold-chain logistics systems, predictive shelf-life models, artificial intelligence-assisted inventory management systems, digital quality-control databases, cloud-connected monitoring platforms, or smart distribution systems configured to further preserve product quality and improve supply-chain traceability.

Although FIG. 15 illustrates one representative protective storage architecture, alternative packaging configurations including unit-dose blister systems, multidose containers, compartmentalized packages, strip packaging, child-resistant packages, senior-friendly packages, recyclable packaging systems, refillable containers, biodegradable packaging systems, smart containers, vacuum cartridges, pressurized containers, and additional preservation-oriented packaging technologies may likewise be employed without departing from the principles of the present invention.

Accordingly, FIG. 15 illustrates a comprehensive protective storage and packaging architecture in which the finished nutraceutical product (1810) is enclosed within the internal protective container (1820), maintained within the controlled atmosphere region (1830), surrounded by the multilayer barrier package (1840), protected by the environmental protection system (1850), transported through the transportation and storage module (1860), monitored by the quality monitoring module (1870), and ultimately accessed through the consumer access module (1880). Collectively, these components establish an integrated preservation system configured to maintain the naturally occurring biochemical profile and elevated phenolic composition of the high-polyphenol olive oil throughout the commercial lifecycle of the nutraceutical product.

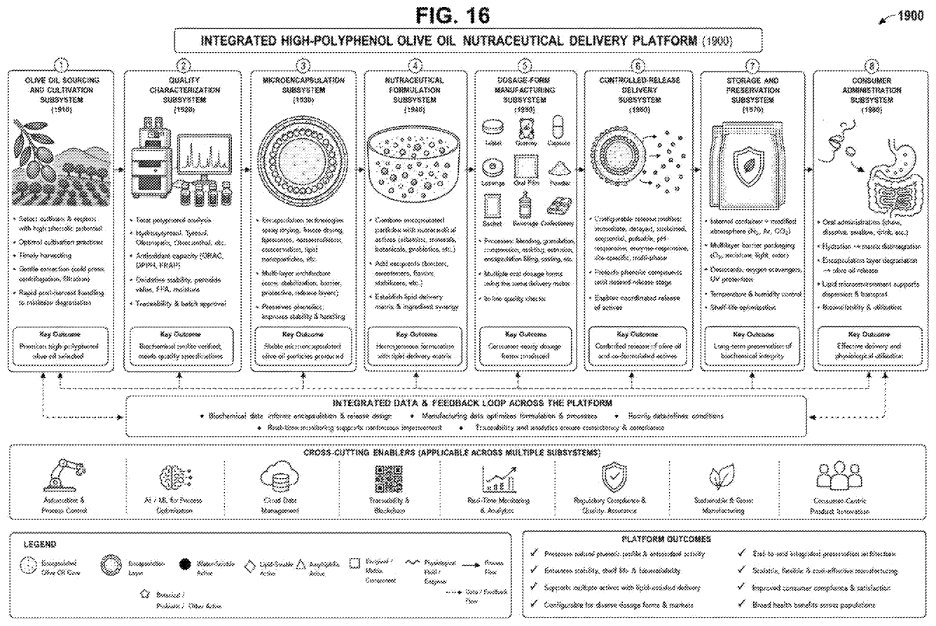

Referring now to FIG. 16, there is illustrated an exemplary integrated high-polyphenol olive oil nutraceutical delivery platform (1900) according to one embodiment of the present invention. The illustrated embodiment represents a comprehensive system architecture integrating the various technologies, manufacturing processes, encapsulation systems, preservation mechanisms, formulation methodologies, dosage-form implementations, and delivery strategies described throughout the present specification. Unlike conventional nutraceutical systems that treat olive oil as a conventional nutritional ingredient or lipid excipient, the present invention establishes an integrated platform in which high-polyphenol olive oil functions as the central biologically active delivery matrix supporting preservation, formulation, controlled release, and coordinated delivery of one or more nutraceutical ingredients.

As illustrated, the integrated nutraceutical delivery platform (1900) generally comprises an olive oil sourcing and cultivation subsystem (1910), a quality characterization subsystem (1920), a microencapsulation subsystem (1930), a nutraceutical formulation subsystem (1940), a dosage-form manufacturing subsystem (1950), a controlled-release delivery subsystem (1960), a storage and preservation subsystem (1970), and a consumer administration subsystem (1980). These subsystems collectively establish an end-to-end technological platform configured to preserve naturally occurring olive oil bioactive compounds while facilitating manufacture and administration of numerous nutraceutical products.

The platform begins with the olive oil sourcing and cultivation subsystem (1910). This subsystem encompasses identification of olive cultivars, agricultural production systems, geographical growing regions, cultivation methodologies, harvesting operations, extraction procedures, and selection of olive oils possessing elevated concentrations of naturally occurring phenolic compounds. In preferred embodiments, the selected olive oil exhibits elevated concentrations of hydroxytyrosol, tyrosol, oleuropein derivatives, oleocanthal, oleacein, ligstroside derivatives, tocopherols, phytosterols, flavonoids, phenolic acids, triterpenes, and additional naturally occurring antioxidant constituents.

The selected olive oil is subsequently processed by the quality characterization subsystem (1920). The quality characterization subsystem performs biochemical evaluation of the selected olive oil through one or more analytical procedures including chromatographic analysis, spectroscopic analysis, antioxidant measurements, compositional analysis, oxidative stability testing, and additional analytical techniques suitable for characterizing the biochemical profile of the olive oil. The resulting biochemical profile may be utilized to determine whether the olive oil satisfies predetermined quality specifications established for the nutraceutical delivery platform.

Olive oil satisfying the desired biochemical criteria proceeds to the microencapsulation subsystem (1930). The microencapsulation subsystem converts the liquid high-polyphenol olive oil into stable encapsulated particles capable of preserving naturally occurring phenolic compounds during manufacturing, storage, transportation, and administration. Representative encapsulation technologies include spray drying, freeze drying, nanoemulsions, microemulsions, liposomes, cyclodextrin complexes, coacervation, protein matrices, hydrocolloid systems, lipid nanoparticles, solid lipid particles, multilayer encapsulation systems, biopolymer encapsulation technologies, and combinations thereof.

In preferred embodiments, the microencapsulation subsystem produces multilayer encapsulated particles comprising an olive oil core surrounded by one or more stabilization layers, barrier layers, protective coatings, and optional controlled-release layers. The resulting encapsulated particles function as structurally stable microreservoirs preserving the naturally occurring biochemical profile of the selected olive oil.

The encapsulated particles are subsequently transferred to the nutraceutical formulation subsystem (1940). The formulation subsystem combines the encapsulated olive oil particles with one or more nutraceutical active ingredients selected according to the intended commercial product. Representative nutraceutical ingredients include vitamins, minerals, botanical extracts, probiotics, prebiotics, amino acids, peptides, phospholipids, omega fatty acids, enzymes, functional fibers, proteins, flavonoids, carotenoids, medicinal mushroom extracts, fermentation products, marine-derived ingredients, trace minerals, adaptogens, and additional functional nutritional compounds.

During formulation, the encapsulated high-polyphenol olive oil particles establish a multifunctional lipid delivery matrix distributed throughout the nutraceutical composition. Unlike conventional excipients that primarily provide structural support, the encapsulated olive oil particles simultaneously function as biologically active ingredients, antioxidant reservoirs, lipid delivery components, controlled-release elements, stabilization systems, and formulation matrices.

The formulated composition proceeds to the dosage-form manufacturing subsystem (1950). The manufacturing subsystem converts the blended formulation into one or more consumer-ready dosage forms including lozenges, fast-dissolving tablets, orally disintegrating tablets, chewable tablets, gummies, oral films, capsules, powders, granules, sachets, beverages, nutritional shots, functional confectionery products, nutritional bars, multiparticulate systems, or additional orally administrable formulations. Manufacturing operations may include blending, granulation, compression, molding, casting, extrusion, capsule filling, drying, coating, or related nutraceutical production processes.

Following manufacture, the dosage forms enter the controlled-release delivery subsystem (1960). The controlled-release subsystem regulates release of the encapsulated high-polyphenol olive oil according to predetermined release characteristics. Representative release mechanisms include immediate release, delayed release, sustained release, sequential release, pulsatile release, site-specific release, pH-responsive release, moisture-responsive release, enzyme-responsive release, multilayer release, and combinations thereof. The controlled-release subsystem substantially preserves the naturally occurring phenolic composition of the olive oil until the desired stage of physiological administration.

Following formulation and controlled-release preparation, the finished dosage forms enter the storage and preservation subsystem (1970). The storage subsystem protects the encapsulated olive oil against degradation during packaging, warehousing, transportation, retail distribution, and consumer storage. Representative preservation technologies include oxygen-barrier packaging, moisture-barrier packaging, inert-gas packaging, multilayer barrier systems, ultraviolet-resistant packaging, desiccants, oxygen scavengers, environmental monitoring systems, smart packaging technologies, quality indicators, digital traceability systems, and combinations thereof.

Throughout storage and distribution, the preservation subsystem substantially minimizes degradation of naturally occurring phenolic compounds while maintaining the structural integrity of the encapsulated olive oil particles. Consequently, the finished product retains its biochemical profile throughout commercial handling prior to administration.

Upon consumer use, the finished product proceeds to the consumer administration subsystem (1980). Administration may occur by oral dissolution, chewing, swallowing, dispersion within liquids, ingestion of capsules, consumption of beverages, or additional oral administration methods depending upon the selected dosage form. Physiological hydration gradually exposes the encapsulated particles, resulting in controlled release of the preserved high-polyphenol olive oil together with associated nutraceutical ingredients.

Following release, the preserved high-polyphenol olive oil establishes a localized lipid microenvironment surrounding co-formulated nutraceutical ingredients. The lipid microenvironment facilitates coordinated dispersion of lipophilic, hydrophilic, and amphiphilic nutraceutical compounds while preserving the naturally occurring biochemical profile of the olive oil throughout the delivery process.

In preferred embodiments, information generated by one subsystem may be communicated to one or more additional subsystems. For example, biochemical characterization data generated by the quality characterization subsystem may influence encapsulation parameters, manufacturing conditions, quality-control procedures, storage specifications, or release characteristics. Likewise, manufacturing quality-control information may be utilized to optimize subsequent production batches.

In certain embodiments, one or more subsystems may incorporate computerized process control, programmable manufacturing equipment, industrial automation systems, laboratory information management systems, manufacturing execution systems, cloud-connected databases, artificial intelligence-assisted quality optimization systems, machine-learning process control algorithms, predictive manufacturing models, digital traceability systems, blockchain verification systems, environmental monitoring systems, or smart packaging technologies. Such technologies may improve manufacturing consistency while maintaining preservation of naturally occurring phenolic compounds.

One significant feature illustrated by FIG. 16 is the integration of agricultural selection, biochemical characterization, encapsulation, formulation, manufacturing, preservation, controlled release, and administration into a unified technological platform. Rather than treating these operations as isolated manufacturing steps, the present invention coordinates each subsystem toward the common objective of preserving and delivering naturally occurring high-polyphenol olive oil bioactive compounds together with one or more nutraceutical ingredients.

Another important aspect illustrated by FIG. 16 is the modular architecture of the overall platform. Individual subsystems may be modified, supplemented, duplicated, omitted, reordered, or combined according to the intended commercial application without departing from the underlying principles of the invention. Likewise, additional encapsulation technologies, manufacturing methodologies, dosage forms, packaging systems, monitoring technologies, or quality-control procedures may be incorporated while preserving the integrated platform architecture.

The integrated platform further enables development of diverse commercial products directed toward cardiovascular wellness, healthy aging, metabolic wellness, immune support, cognitive support, gastrointestinal wellness, sports nutrition, pediatric nutrition, geriatric nutrition, personalized nutrition, veterinary nutrition, medical nutrition, and general dietary supplementation while utilizing substantially the same underlying high-polyphenol olive oil delivery technology.

Accordingly, FIG. 16 illustrates a comprehensive integrated high-polyphenol olive oil nutraceutical delivery platform in which the olive oil sourcing and cultivation subsystem (1910), quality characterization subsystem (1920), microencapsulation subsystem (1930), nutraceutical formulation subsystem (1940), dosage-form manufacturing subsystem (1950), controlled-release delivery subsystem (1960), storage and preservation subsystem (1970), and consumer administration subsystem (1980) collectively establish an end-to-end technological ecosystem configured to preserve, formulate, manufacture, protect, and deliver high-polyphenol olive oil together with one or more nutraceutical ingredients in a wide variety of consumer-ready oral dosage forms. The integrated platform provides improved preservation of naturally occurring phenolic compounds, enhanced manufacturing flexibility, broad dosage-form compatibility, configurable release characteristics, and long-term commercial stability while maintaining the multifunctional biological and structural characteristics of the high-polyphenol olive oil delivery matrix.

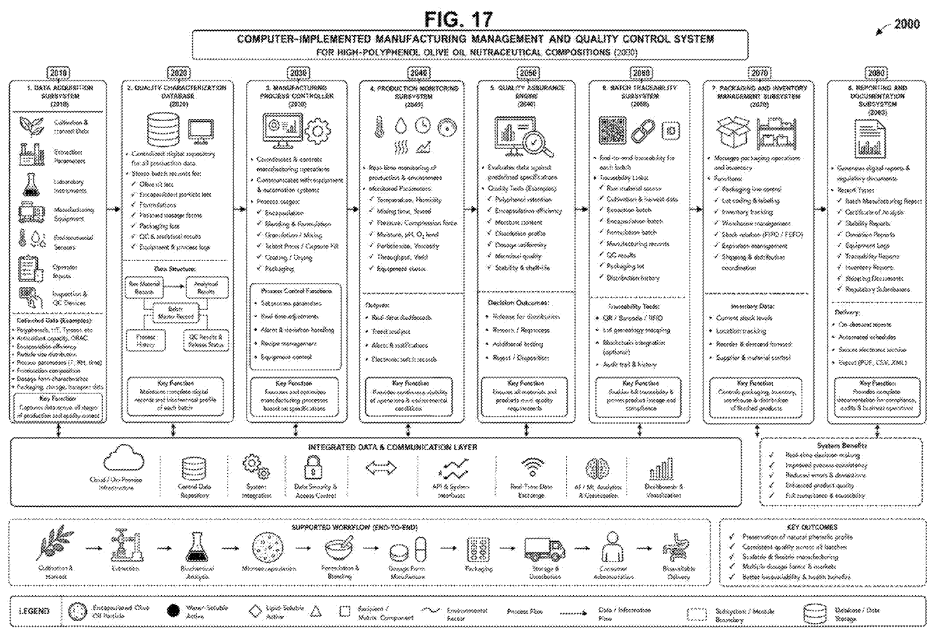

Referring now to FIG. 17, there is illustrated an exemplary computer-implemented manufacturing management and quality control system (2000) configured to support production of nutraceutical compositions incorporating the high-polyphenol olive oil delivery platform according to one embodiment of the present invention. The illustrated embodiment provides a digital process management architecture configured to coordinate sourcing, biochemical characterization, encapsulation, formulation, manufacturing, packaging, quality assurance, traceability, and inventory management while preserving the naturally occurring biochemical characteristics of the selected high-polyphenol olive oil throughout commercial production.

As illustrated, the manufacturing management and quality control system (2000) generally comprises a data acquisition subsystem (2010), a quality characterization database (2020), a manufacturing process controller (2030), a production monitoring subsystem (2040), a quality assurance engine (2050), a batch traceability subsystem (2060), a packaging and inventory management subsystem (2070), and a reporting and documentation subsystem (2080). Collectively, these components establish an integrated digital infrastructure configured to support consistent manufacture of nutraceutical products utilizing the high-polyphenol olive oil delivery platform.

The process begins with the data acquisition subsystem (2010). The data acquisition subsystem receives information associated with one or more manufacturing stages including olive cultivation, harvesting, extraction, biochemical characterization, encapsulation, formulation, dosage-form manufacturing, packaging, storage, transportation, and quality testing. Information may be obtained from laboratory instruments, manufacturing equipment, environmental sensors, operator interfaces, enterprise software systems, automated production lines, handheld inspection devices, or combinations thereof.

Representative information acquired by the data acquisition subsystem includes olive cultivar identification, geographical origin, harvest date, extraction conditions, total polyphenol concentration, hydroxytyrosol concentration, tyrosol concentration, oleuropein concentration, oleocanthal concentration, oleacein concentration, tocopherol concentration, antioxidant capacity, peroxide value, moisture content, encapsulation efficiency, particle-size distribution, formulation composition, dosage-form characteristics, packaging information, storage conditions, and additional manufacturing parameters associated with production of the nutraceutical composition.

The acquired manufacturing information is stored within the quality characterization database (2020). The database maintains digital records associated with individual olive oil batches, encapsulated particle batches, intermediate formulations, finished dosage forms, packaging lots, quality-control results, production histories, and related manufacturing information. The database may be implemented using relational databases, distributed databases, cloud-based storage systems, enterprise resource planning systems, laboratory information management systems, manufacturing execution systems, or combinations thereof.

In preferred embodiments, each production batch is assigned a unique digital record containing biochemical characterization data associated with the selected high-polyphenol olive oil. The stored biochemical profile enables verification that the finished nutraceutical composition originates from olive oil satisfying predetermined quality specifications established for the delivery platform.

Information stored within the quality characterization database is supplied to the manufacturing process controller (2030). The manufacturing process controller coordinates one or more production operations including encapsulation, formulation, blending, particle conditioning, dosage-form production, coating, packaging, storage, or additional manufacturing activities. In certain embodiments, the controller communicates with programmable manufacturing equipment, industrial controllers, automated production lines, robotic handling systems, weighing equipment, mixing systems, encapsulation equipment, tablet presses, capsule fillers, packaging systems, and environmental control equipment.

During production, the manufacturing process controller may monitor representative process variables including processing temperature, humidity, mixing duration, compression force, encapsulation parameters, drying conditions, coating thickness, oxygen concentration, moisture levels, production throughput, particle-size distribution, and formulation homogeneity. The controller may compare measured values with predetermined manufacturing specifications and generate notifications if one or more parameters deviate from acceptable operating ranges.

The manufacturing process further includes the production monitoring subsystem (2040). The production monitoring subsystem continuously or periodically receives operational information from manufacturing equipment and associated environmental sensors. Representative monitored parameters include equipment status, production rates, environmental conditions, process timing, raw material consumption, formulation consistency, packaging integrity, and equipment maintenance status.

In certain embodiments, the production monitoring subsystem generates operational records documenting completion of individual manufacturing operations. Such records may include timestamps, equipment identifiers, operator identifiers, production lot numbers, process conditions, environmental measurements, inspection results, and additional production information suitable for manufacturing documentation and regulatory compliance.

The manufacturing management system further comprises the quality assurance engine (2050). The quality assurance engine evaluates analytical data obtained throughout manufacturing to determine whether intermediate materials and finished products satisfy predetermined quality specifications. Representative quality evaluations include verification of phenolic concentrations, encapsulation efficiency, dosage uniformity, moisture content, antioxidant activity, dissolution characteristics, microbial quality, packaging integrity, oxidative stability, and additional analytical measurements relevant to the finished nutraceutical composition.

Products satisfying the predetermined quality specifications may be released for packaging and commercial distribution, whereas products failing to satisfy one or more quality criteria may be quarantined, reprocessed, rejected, subjected to additional testing, or otherwise managed according to predetermined manufacturing protocols.

The illustrated system additionally comprises the batch traceability subsystem (2060). The batch traceability subsystem associates each finished nutraceutical product with the corresponding olive oil source, biochemical characterization data, encapsulation process, formulation batch, manufacturing history, quality-control records, packaging information, and distribution records. Such traceability facilitates verification of production history throughout the commercial lifecycle of the product.

In preferred embodiments, the batch traceability subsystem generates digital production records enabling identification of raw material sources, processing conditions, analytical results, packaging lots, storage histories, and distribution channels associated with individual commercial products. Traceability information may be stored electronically, encoded within machine-readable identifiers, or associated with enterprise quality-control systems.

The finished products subsequently enter the packaging and inventory management subsystem (2070). This subsystem manages packaging operations, inventory control, warehouse storage, shipping preparation, product identification, lot management, expiration-date assignment, stock rotation, and commercial distribution. Inventory records may be updated automatically as products move through manufacturing, storage, transportation, and customer fulfillment operations.

The manufacturing management system further comprises the reporting and documentation subsystem (2080). The reporting subsystem generates production reports, quality-control reports, certificates of analysis, manufacturing summaries, batch records, inventory reports, traceability reports, packaging records, distribution documentation, regulatory documentation, and additional reports associated with production of the nutraceutical compositions. Such reports may be generated automatically or upon request by manufacturing personnel.

In various embodiments, the manufacturing management system may further incorporate network communication interfaces permitting secure exchange of manufacturing information among production facilities, analytical laboratories, distribution centers, inventory systems, enterprise software platforms, cloud computing environments, or remote quality-control facilities.

In certain embodiments, one or more functions performed by the manufacturing management and quality control system may be assisted by statistical analysis, predictive process models, rule-based process control, automated quality evaluation, machine-learning algorithms, artificial intelligence-assisted optimization, anomaly detection systems, digital twin models, or predictive maintenance systems. Such computational techniques may improve manufacturing consistency while preserving the naturally occurring biochemical characteristics of the selected high-polyphenol olive oil. The invention, however, is not limited to implementation using any particular computational methodology.

One significant feature illustrated by FIG. 17 is the integration of biochemical characterization data with downstream manufacturing operations. Rather than treating laboratory analysis and production as independent activities, the illustrated architecture enables manufacturing decisions to be informed by measured biochemical characteristics of the selected olive oil, thereby supporting consistent implementation of the high-polyphenol olive oil delivery platform.

Another important aspect illustrated by FIG. 17 is end-to-end batch traceability. Each finished nutraceutical product may be digitally associated with its corresponding raw material source, encapsulation process, formulation history, manufacturing parameters, quality-control records, packaging information, and distribution history. Such traceability enhances manufacturing oversight, quality assurance, inventory management, and commercial product documentation.

Although FIG. 17 illustrates one representative computer-implemented manufacturing management architecture, individual functional modules may be combined, subdivided, omitted, duplicated, or supplemented with additional process-control systems, analytical instrumentation, manufacturing equipment, communication networks, cloud computing resources, enterprise software applications, regulatory documentation systems, or digital quality-management technologies without departing from the principles of the present invention.

Accordingly, FIG. 17 illustrates a comprehensive computer-implemented manufacturing management and quality control system in which the data acquisition subsystem (2010), quality characterization database (2020), manufacturing process controller (2030), production monitoring subsystem (2040), quality assurance engine (2050), batch traceability subsystem (2060), packaging and inventory management subsystem (2070), and reporting and documentation subsystem (2080) collectively establish an integrated digital manufacturing architecture configured to support production, quality assurance, traceability, documentation, and commercial distribution of nutraceutical products incorporating the high-polyphenol olive oil delivery platform while maintaining preservation of the naturally occurring phenolic composition and biochemical integrity of the selected olive oil.

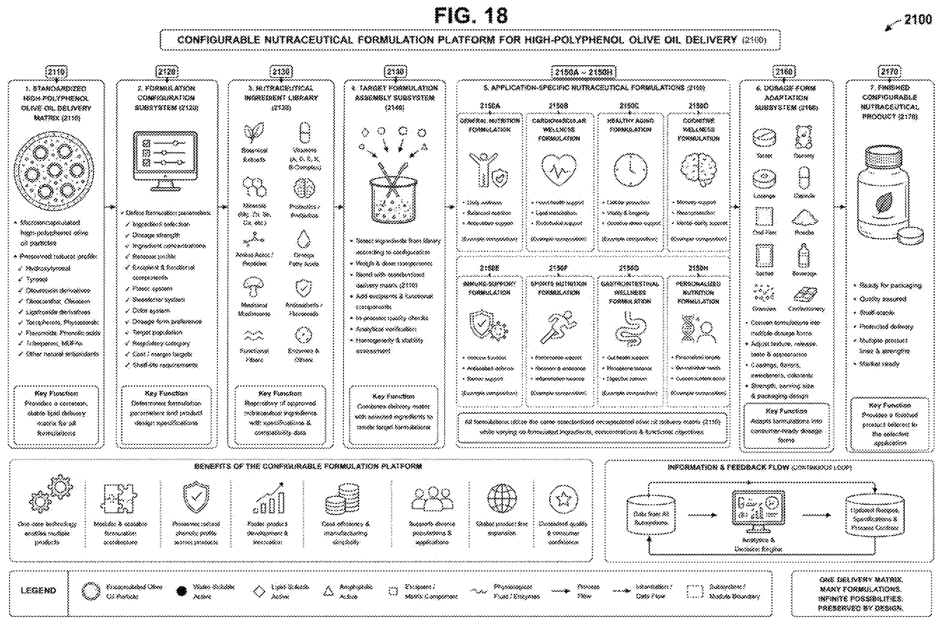

Referring now to FIG. 18, there is illustrated an exemplary configurable nutraceutical formulation platform (2100) according to one embodiment of the present invention. The illustrated embodiment demonstrates how the high-polyphenol olive oil delivery platform may serve as a common formulation architecture capable of generating multiple nutraceutical compositions configured for different nutritional objectives, consumer populations, dosage strengths, ingredient combinations, and commercial product categories. Unlike conventional nutraceutical products that are generally developed as independent formulations for individual applications, the present invention establishes a modular formulation platform in which a common encapsulated high-polyphenol olive oil delivery matrix may be selectively combined with different nutraceutical ingredient profiles while preserving the naturally occurring biochemical characteristics of the selected olive oil.

As illustrated, the configurable nutraceutical formulation platform (2100) generally comprises a standardized high-polyphenol olive oil delivery matrix (2110), a formulation configuration subsystem (2120), a nutraceutical ingredient library (2130), a target formulation assembly subsystem (2140), a plurality of application-specific nutraceutical formulations (2150A-2150H), a dosage-form adaptation subsystem (2160), and a finished configurable nutraceutical product (2170). Collectively, these functional components enable development of numerous nutraceutical formulations utilizing substantially the same underlying delivery platform.

The platform begins with the standardized high-polyphenol olive oil delivery matrix (2110). The delivery matrix comprises a plurality of microencapsulated high-polyphenol olive oil particles produced according to the encapsulation technologies previously described throughout the specification. The olive oil contained within the delivery matrix possesses a preserved biochemical profile including naturally occurring hydroxytyrosol, tyrosol, oleuropein derivatives, oleocanthal, oleacein, ligstroside derivatives, tocopherols, phytosterols, flavonoids, phenolic acids, triterpenes, and additional naturally occurring antioxidant constituents.

In preferred embodiments, the standardized delivery matrix remains substantially constant among multiple product formulations. Consequently, different commercial nutraceutical products may share substantially the same encapsulated olive oil technology while differing primarily in the selection and concentration of accompanying nutraceutical ingredients.

The standardized delivery matrix is supplied to the formulation configuration subsystem (2120). The formulation configuration subsystem determines one or more formulation parameters including ingredient selection, ingredient concentration, dosage strength, excipient composition, release profile, dosage-form selection, flavor profile, sweetener system, coloring agents, coating materials, and additional formulation characteristics appropriate for the intended commercial product.

In certain embodiments, the formulation configuration subsystem utilizes predetermined formulation specifications corresponding to different nutritional product categories. Alternatively, formulation parameters may be selected according to manufacturing requirements, market demands, product differentiation strategies, regulatory requirements, consumer preferences, or combinations thereof.

The formulation configuration subsystem communicates with the nutraceutical ingredient library (2130). The ingredient library comprises a collection of nutraceutical components available for incorporation into the standardized delivery matrix. Representative ingredient categories include vitamins, minerals, botanical extracts, medicinal plant derivatives, probiotics, prebiotics, amino acids, peptides, enzymes, phospholipids, omega fatty acids, proteins, functional fibers, medicinal mushroom extracts, flavonoids, carotenoids, fermentation products, marine-derived ingredients, trace minerals, adaptogens, antioxidants, and additional nutraceutical materials.

Each ingredient contained within the nutraceutical ingredient library may be stored as an individual formulation component together with associated manufacturing information including ingredient identity, concentration range, formulation compatibility, stability information, processing requirements, particle characteristics, encapsulation status, storage conditions, and quality-control specifications. Such information facilitates systematic assembly of multiple nutraceutical formulations while maintaining manufacturing consistency.

Selected ingredients are subsequently transferred to the target formulation assembly subsystem (2140). During formulation assembly, one or more nutraceutical ingredients are combined with the standardized high-polyphenol olive oil delivery matrix to generate a desired finished formulation. Representative formulation operations include weighing, blending, granulation, encapsulation, compression, molding, extrusion, coating, or additional manufacturing processes appropriate for the selected dosage form.

The formulation assembly subsystem may generate numerous application-specific nutraceutical formulations (2150A-2150H). Although illustrated as eight representative formulation categories, the invention is not limited to any particular number of formulations. Representative formulation categories may include:
(2150A) General nutritional supplementation formulations;
(2150B) Cardiovascular wellness formulations;
(2150C) Healthy aging formulations;
(2150D) Cognitive wellness formulations;
(2150E) Immune-support formulations;
(2150F) Sports nutrition formulations;
(2150G) Gastrointestinal wellness formulations; and
(2150H) Personalized nutritional formulations.

These categories are presented solely as representative commercial examples and are not intended to limit the scope of the invention or imply therapeutic efficacy for any particular medical condition.

In preferred embodiments, each application-specific formulation incorporates substantially the same standardized encapsulated high-polyphenol olive oil delivery matrix while differing primarily in the identity, concentration, relative proportions, and combinations of the co-formulated nutraceutical ingredients. Consequently, manufacturing complexity may be reduced while permitting development of multiple commercial products utilizing a common technological platform.

The assembled formulation subsequently proceeds to the dosage-form adaptation subsystem (2160). The dosage-form adaptation subsystem converts the assembled formulation into one or more finished dosage forms including lozenges, fast-dissolving tablets, orally disintegrating tablets, chewable tablets, gummies, capsules, powders, granules, sachets, oral films, beverages, nutritional shots, functional confectionery products, nutritional bars, multiparticulate formulations, and additional orally administrable dosage forms described throughout the present specification.

In certain embodiments, the dosage-form adaptation subsystem modifies one or more formulation characteristics including tablet hardness, chewability, dissolution rate, flavor profile, sweetness level, mouthfeel, release profile, particle distribution, coating composition, package size, dosage strength, serving size, or additional product attributes according to the intended consumer application while preserving the underlying encapsulated olive oil delivery matrix.

Following dosage-form adaptation, the formulation becomes a finished configurable nutraceutical product (2170). The finished product may subsequently undergo quality assurance, protective packaging, storage, transportation, commercial distribution, and consumer administration according to the preservation methodologies previously described throughout the specification.

One significant feature illustrated by FIG. 18 is that the high-polyphenol olive oil delivery matrix functions as a universal formulation platform rather than a formulation-specific ingredient. By maintaining a common encapsulated olive oil architecture while selectively varying accompanying nutraceutical ingredients, manufacturers may efficiently develop numerous commercial products utilizing substantially the same core delivery technology.

Another important aspect illustrated by FIG. 18 is the modular nature of the formulation platform. Individual nutraceutical ingredients may be added, removed, substituted, duplicated, or reformulated without requiring redesign of the encapsulated high-polyphenol olive oil delivery matrix itself. This modular architecture facilitates product line expansion, reformulation, dosage customization, and future product development while preserving the underlying technological advantages of the invention.

In various embodiments, the formulation configuration subsystem may further incorporate computerized formulation software, recipe management systems, formulation databases, manufacturing execution systems, statistical formulation optimization, predictive compatibility analysis, rule-based formulation engines, artificial intelligence-assisted formulation design, machine-learning optimization systems, or cloud-connected product development platforms. Such computational tools may facilitate efficient development of multiple nutraceutical formulations while maintaining manufacturing consistency and preservation of the naturally occurring phenolic composition of the selected olive oil.

Furthermore, although FIG. 18 illustrates representative nutraceutical formulation categories, the configurable platform may be adapted to additional consumer applications including pediatric nutrition, geriatric nutrition, prenatal nutrition, medical nutrition, veterinary nutrition, clinical nutrition, personalized dietary supplementation, functional foods, sports beverages, wellness products, and additional nutraceutical product categories without departing from the principles of the present invention.

Accordingly, FIG. 18 illustrates a configurable nutraceutical formulation platform in which the standardized high-polyphenol olive oil delivery matrix (2110) cooperates with the formulation configuration subsystem (2120), nutraceutical ingredient library (2130), target formulation assembly subsystem (2140), application-specific nutraceutical formulations (2150A-2150H), and dosage-form adaptation subsystem (2160) to produce a finished configurable nutraceutical product (2170). Collectively, these components establish a modular formulation architecture capable of generating multiple nutraceutical products while preserving the naturally occurring biochemical profile, encapsulation technology, and multifunctional delivery characteristics of the high-polyphenol olive oil delivery platform.

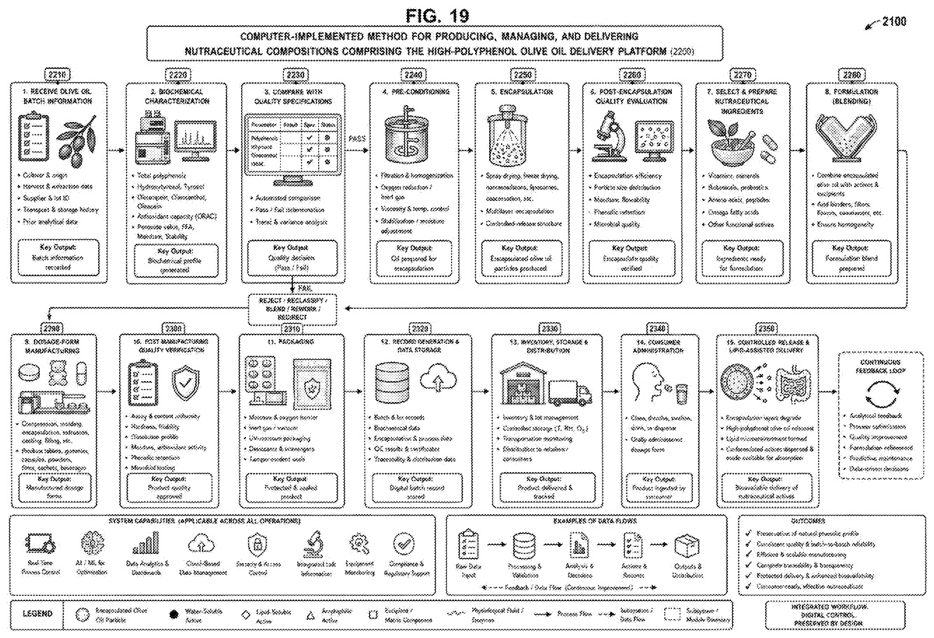

Referring now to FIG. 19, there is illustrated an exemplary computer-implemented method (2200) for producing, managing, and delivering nutraceutical compositions comprising the high-polyphenol olive oil delivery platform according to one embodiment of the present invention. The illustrated embodiment represents a logical operational workflow executed through coordinated interaction among manufacturing equipment, analytical systems, quality-control systems, computerized process management systems, and production personnel. The method provides a systematic framework for preserving the naturally occurring biochemical characteristics of high-polyphenol olive oil while producing consistent nutraceutical formulations suitable for commercial distribution.

The computer-implemented method (2200) begins at operation (2210) in which information associated with one or more candidate olive oil batches is received. Representative information includes olive cultivar identification, geographical origin, agricultural production method, harvest information, extraction method, storage history, supplier identification, transportation records, and previously obtained analytical information. The received information may be manually entered, electronically transmitted, automatically collected from manufacturing equipment, or retrieved from one or more production databases.

At operation (2220), the received olive oil undergoes biochemical characterization. One or more analytical instruments determine representative biochemical characteristics including total polyphenol concentration, hydroxytyrosol concentration, tyrosol concentration, oleuropein concentration, oleocanthal concentration, oleacein concentration, tocopherol concentration, phytosterol concentration, antioxidant capacity, oxidative stability, peroxide value, moisture content, fatty acid profile, and additional quality-related parameters described throughout the present specification.

At operation (2230), the measured biochemical characteristics are compared with predetermined quality specifications stored within one or more quality-control databases or manufacturing control systems. The comparison may determine whether the evaluated olive oil satisfies predefined manufacturing criteria established for production of the high-polyphenol olive oil delivery platform.

If the evaluated olive oil satisfies the predetermined quality specifications, the method proceeds to operation (2240). If one or more quality criteria are not satisfied, the corresponding production batch may be rejected, reclassified, subjected to additional testing, blended with additional olive oils, redirected to alternative manufacturing operations, or otherwise processed according to predetermined production procedures.

At operation (2240), the approved olive oil undergoes one or more pre-conditioning operations. Representative pre-conditioning operations include homogenization, filtration, emulsification, oxygen reduction, inert-gas conditioning, viscosity adjustment, particle-size modification, temperature conditioning, stabilization, surfactant incorporation, moisture adjustment, and additional preparatory processes suitable for encapsulation.

Following preparation, the method proceeds to operation (2250) in which the approved high-polyphenol olive oil is encapsulated. Representative encapsulation technologies include spray drying, freeze drying, liposomal encapsulation, nanoemulsions, microemulsions, cyclodextrin inclusion complexes, coacervation, protein matrices, polysaccharide matrices, lipid nanoparticles, solid lipid particles, hydrocolloid encapsulation systems, multilayer encapsulation systems, or combinations thereof. The encapsulation operation produces stable particulate structures configured to preserve the naturally occurring phenolic composition of the olive oil.

At operation (2260), the encapsulated particles undergo post-encapsulation quality evaluation. Representative quality evaluations include determination of encapsulation efficiency, particle-size distribution, moisture content, oxidative stability, phenolic retention, dissolution characteristics, flowability, compressibility, microbial quality, and additional analytical measurements associated with production of the encapsulated delivery matrix.

Encapsulated particles satisfying the desired quality specifications proceed to operation (2270), in which one or more nutraceutical ingredients are selected and prepared for formulation. Representative nutraceutical ingredients include vitamins, minerals, botanical extracts, probiotics, prebiotics, amino acids, peptides, phospholipids, omega fatty acids, functional fibers, medicinal mushroom extracts, antioxidants, enzymes, proteins, flavonoids, carotenoids, marine-derived ingredients, trace minerals, adaptogens, and combinations thereof.

At operation (2280), the encapsulated olive oil particles are combined with the selected nutraceutical ingredients to produce a formulation mixture. The formulation process may further incorporate excipients including binders, fillers, sweeteners, lubricants, disintegrants, stabilizers, flavoring agents, coloring agents, preservatives, processing aids, coating materials, or additional formulation components according to the desired dosage form.

The formulation subsequently proceeds to operation (2290), in which one or more consumer-ready dosage forms are manufactured. Representative dosage forms include lozenges, fast-dissolving tablets, orally disintegrating tablets, chewable tablets, gummies, capsules, powders, granules, oral films, sachets, nutritional beverages, nutritional shots, functional confectionery products, multiparticulate formulations, and additional oral nutraceutical products.

At operation (2300), the manufactured dosage forms undergo post-manufacturing quality verification. Representative quality evaluations include dosage uniformity, tablet hardness, friability, dissolution profile, moisture content, antioxidant activity, phenolic retention, microbial testing, packaging compatibility, oxidative stability, shelf-life evaluation, and additional quality-control analyses.

Following quality verification, the method proceeds to operation (2310) in which the finished dosage forms are packaged within protective packaging systems. Representative packaging technologies include moisture-barrier packaging, oxygen-barrier packaging, inert-gas packaging, multilayer laminate packaging, ultraviolet-resistant packaging, blister packaging, desiccant-assisted packaging, smart packaging systems, tamper-evident packaging, and additional preservation-oriented packaging architectures configured to maintain the naturally occurring biochemical profile of the encapsulated olive oil.

At operation (2320), production records associated with the finished product are generated and stored. Representative production records include batch identification, biochemical characterization data, encapsulation parameters, formulation composition, manufacturing conditions, analytical results, packaging information, storage history, traceability records, inventory information, quality-control documentation, and distribution records. The production records may be stored within local databases, enterprise manufacturing systems, cloud-based information systems, laboratory information management systems, manufacturing execution systems, or combinations thereof.

The packaged product subsequently proceeds to operation (2330) in which inventory management, warehouse storage, transportation, retail distribution, or direct consumer fulfillment is performed. During these operations, environmental conditions including temperature, humidity, oxygen exposure, storage duration, transportation duration, and package integrity may be monitored to preserve the naturally occurring phenolic composition of the encapsulated olive oil.

At operation (2340), the finished nutraceutical product is administered by a consumer through oral ingestion. Depending upon the selected dosage form, administration may comprise swallowing, chewing, oral dissolution, dispersion within a beverage, ingestion of capsules, consumption of nutritional beverages, or other oral administration methods.

Following administration, the method proceeds to operation (2350) in which the encapsulated high-polyphenol olive oil undergoes controlled release according to the selected encapsulation architecture. The encapsulated particles gradually release the preserved olive oil while substantially maintaining the naturally occurring concentrations of hydroxytyrosol, tyrosol, oleuropein derivatives, oleocanthal, oleacein, ligstroside derivatives, tocopherols, phytosterols, flavonoids, phenolic acids, triterpenes, and additional naturally occurring antioxidant constituents.

Released olive oil establishes a localized lipid delivery environment surrounding one or more co-formulated nutraceutical ingredients. The resulting lipid microenvironment facilitates coordinated dispersion of hydrophilic, amphiphilic, and lipophilic nutraceutical ingredients according to the delivery principles previously described throughout the present specification.

In certain embodiments, one or more operations illustrated in FIG. 19 may be executed under computerized supervision. Production scheduling systems, programmable manufacturing equipment, statistical process-control systems, digital quality-management systems, laboratory information management systems, enterprise resource planning systems, cloud-based manufacturing systems, artificial intelligence-assisted production optimization systems, machine-learning quality-control algorithms, digital twin manufacturing models, or combinations thereof may assist in execution of one or more operations while maintaining preservation of the naturally occurring biochemical profile of the selected olive oil.

One significant feature illustrated by FIG. 19 is the integration of biochemical characterization, manufacturing, quality assurance, packaging, traceability, and administration into a unified operational methodology. Rather than treating these activities as isolated production operations, the present invention coordinates each operation toward preservation of the naturally occurring high-polyphenol olive oil throughout the complete manufacturing and commercial lifecycle.

Another important aspect illustrated by FIG. 19 is that the computer-implemented method remains adaptable to numerous manufacturing environments. Individual operations may be automated, manually performed, omitted, reordered, repeated, subdivided, supplemented, or executed in parallel depending upon manufacturing capacity, production technology, commercial requirements, regulatory requirements, or product-specific considerations without departing from the principles of the invention.

Furthermore, although the illustrated embodiment describes representative manufacturing operations, additional analytical procedures, encapsulation technologies, formulation methodologies, dosage-form manufacturing techniques, packaging architectures, digital manufacturing systems, quality-control algorithms, or computerized optimization techniques may likewise be incorporated within the scope of the present invention.

Accordingly, FIG. 19 illustrates a comprehensive computer-implemented operational method in which operations (2210) through (2350) collectively establish an integrated workflow for selecting, characterizing, encapsulating, formulating, manufacturing, packaging, documenting, distributing, and administering nutraceutical compositions incorporating the high-polyphenol olive oil delivery platform. The illustrated method enables consistent preservation of the naturally occurring biochemical profile of the selected olive oil while supporting efficient commercial production, digital quality management, comprehensive traceability, and controlled consumer delivery of the resulting nutraceutical products.

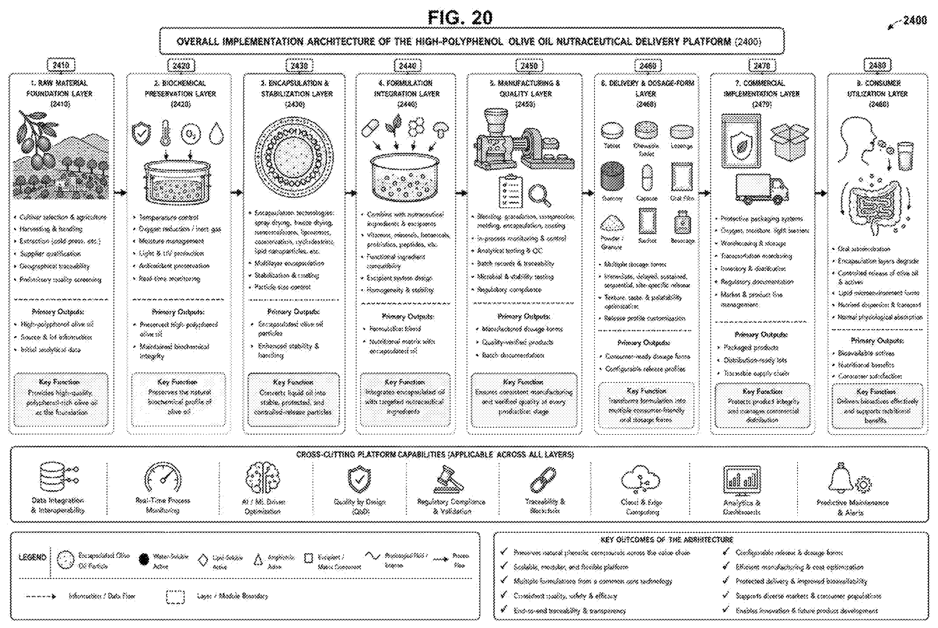

Referring now to FIG. 20, there is illustrated an exemplary overall implementation architecture (2400) of the high-polyphenol olive oil nutraceutical delivery platform according to one embodiment of the present invention. The illustrated embodiment provides a generalized representation of the invention as an integrated technological platform extending from raw material selection through commercial product implementation. Unlike conventional nutraceutical technologies that are directed toward isolated compositions or individual dosage forms, the present invention establishes a comprehensive implementation architecture capable of supporting numerous manufacturing processes, encapsulation technologies, formulation strategies, dosage forms, preservation systems, and commercial product configurations while maintaining the naturally occurring biochemical characteristics of high-polyphenol olive oil.

As illustrated, the implementation architecture (2400) generally comprises a raw material foundation layer (2410), a biochemical preservation layer (2420), an encapsulation and stabilization layer (2430), a formulation integration layer (2440), a manufacturing and quality layer (2450), a delivery and dosage-form layer (2460), a commercial implementation layer (2470), and a consumer utilization layer (2480). These implementation layers cooperate to establish a scalable and modular nutraceutical delivery ecosystem capable of supporting a broad range of commercial applications.

The implementation architecture begins with the raw material foundation layer (2410). This layer encompasses identification, cultivation, harvesting, extraction, sourcing, and selection of olive oils exhibiting desirable biochemical characteristics. Representative considerations include olive cultivar selection, agricultural production methods, geographical origin, environmental growing conditions, harvesting procedures, extraction technologies, storage conditions, and quality characterization. In preferred embodiments, the raw material foundation layer provides olive oils containing elevated concentrations of naturally occurring hydroxytyrosol, tyrosol, oleuropein derivatives, oleocanthal, oleacein, ligstroside derivatives, tocopherols, phytosterols, flavonoids, phenolic acids, triterpenes, and additional naturally occurring antioxidant constituents.

Information and materials generated within the raw material foundation layer are supplied to the biochemical preservation layer (2420). The biochemical preservation layer is configured to substantially maintain the naturally occurring biochemical profile of the selected olive oil throughout subsequent manufacturing operations. Representative preservation mechanisms include temperature management, oxygen reduction, inert atmosphere processing, moisture control, ultraviolet protection, controlled storage conditions, antioxidant preservation methodologies, and quality monitoring systems. Preservation of naturally occurring phenolic compounds represents a foundational characteristic of the present invention.

The preserved olive oil subsequently proceeds to the encapsulation and stabilization layer (2430). The encapsulation and stabilization layer converts the liquid olive oil into stable particulate structures configured for incorporation into orally administrable nutraceutical formulations. Representative encapsulation technologies include spray drying, freeze drying, coacervation, nanoemulsions, microemulsions, liposomes, cyclodextrin complexes, hydrocolloid encapsulation, protein matrices, polysaccharide matrices, lipid nanoparticles, solid lipid particles, multilayer encapsulation architectures, and hybrid encapsulation systems. The encapsulation layer substantially preserves the naturally occurring phenolic composition while improving storage stability, manufacturing compatibility, and controlled release.

Encapsulated particles generated by the encapsulation layer are transferred to the formulation integration layer (2440). Within this layer, the encapsulated high-polyphenol olive oil particles are combined with one or more nutraceutical ingredients selected according to the desired commercial formulation. Representative nutraceutical ingredients include vitamins, minerals, botanical extracts, probiotics, prebiotics, amino acids, peptides, phospholipids, omega fatty acids, proteins, functional fibers, enzymes, medicinal mushroom extracts, flavonoids, carotenoids, marine-derived ingredients, adaptogens, trace minerals, antioxidants, and additional nutraceutical compounds. The encapsulated olive oil particles establish a multifunctional delivery matrix supporting compatibility among chemically diverse formulation ingredients.

Following formulation, the resulting compositions proceed to the manufacturing and quality layer (2450). This layer encompasses blending, granulation, compression, molding, coating, drying, encapsulation, dosage-form manufacturing, packaging, quality assurance, analytical testing, traceability, documentation, inventory management, environmental monitoring, and additional manufacturing operations associated with commercial production. Representative analytical evaluations include verification of phenolic concentrations, encapsulation efficiency, dosage uniformity, oxidative stability, dissolution characteristics, moisture content, antioxidant activity, packaging integrity, microbial quality, and shelf-life assessment.

Manufactured products subsequently proceed to the delivery and dosage-form layer (2460). The delivery layer supports implementation of the encapsulated high-polyphenol olive oil platform within numerous oral dosage forms including lozenges, fast-dissolving tablets, orally disintegrating tablets, chewable tablets, gummies, oral films, capsules, powders, granules, sachets, beverages, nutritional shots, functional confectionery products, multiparticulate systems, nutritional bars, and additional edible formulations. The delivery layer further supports implementation of immediate-release, delayed-release, sustained-release, sequential-release, site-specific-release, enzyme-responsive, moisture-responsive, and pH-responsive delivery mechanisms.

Finished products generated by the delivery layer proceed to the commercial implementation layer (2470). The commercial implementation layer encompasses product packaging, warehousing, transportation, distribution, inventory management, retail commercialization, e-commerce fulfillment, regulatory documentation, batch traceability, quality documentation, marketing support, and post-manufacturing quality preservation. Representative packaging technologies include multilayer barrier systems, oxygen-resistant packaging, moisture-resistant packaging, inert-gas packaging, ultraviolet-resistant packaging, smart packaging systems, and environmental monitoring technologies configured to preserve the biochemical integrity of the encapsulated olive oil throughout the commercial lifecycle.

Commercially distributed products ultimately reach the consumer utilization layer (2480). Within this layer, consumers administer the nutraceutical compositions through conventional oral administration routes appropriate for the selected dosage form. Controlled release of the encapsulated olive oil establishes a localized lipid delivery environment that supports coordinated dispersion of co-formulated nutraceutical ingredients while substantially maintaining the naturally occurring biochemical profile preserved throughout manufacturing.

In preferred embodiments, information generated within each implementation layer may be communicated to one or more additional layers. For example, biochemical characterization data generated within the raw material foundation layer may influence encapsulation parameters, manufacturing specifications, dosage-form selection, packaging requirements, quality-control criteria, or commercial implementation strategies. Likewise, manufacturing data generated during commercial production may be utilized to improve subsequent production cycles through continuous quality improvement methodologies.

One significant feature illustrated by FIG. 20 is the layered implementation architecture of the present invention. Each implementation layer performs specialized functions while simultaneously contributing to the common objective of preserving and delivering naturally occurring high-polyphenol olive oil within diverse nutraceutical formulations. The layered architecture enables independent optimization of individual manufacturing operations without requiring redesign of the overall delivery platform.

Another important aspect illustrated by FIG. 20 is the scalability of the implementation architecture. The platform may be implemented within laboratory-scale manufacturing, pilot-scale production, commercial manufacturing facilities, contract manufacturing organizations, automated continuous manufacturing systems, or distributed production environments while preserving the underlying principles of the invention. Individual implementation layers may be expanded, subdivided, duplicated, automated, or integrated with additional manufacturing technologies according to production requirements.

In certain embodiments, one or more implementation layers may incorporate computerized process control, industrial automation systems, laboratory information management systems, manufacturing execution systems, cloud-based manufacturing platforms, enterprise resource planning systems, statistical process-control methodologies, digital quality management systems, predictive manufacturing models, machine-learning process optimization, artificial intelligence-assisted formulation development, blockchain traceability systems, smart packaging technologies, digital twin manufacturing environments, or combinations thereof. These computational technologies may improve manufacturing consistency while preserving the naturally occurring biochemical profile of the selected high-polyphenol olive oil.

The implementation architecture further supports development of multiple commercial product families utilizing substantially the same underlying delivery platform. Representative product categories include general dietary supplements, wellness formulations, sports nutrition products, healthy aging products, cognitive wellness formulations, immune-support products, gastrointestinal wellness formulations, functional foods, personalized nutrition systems, veterinary nutritional products, medical nutrition formulations, and additional nutraceutical products. These examples represent implementation environments for the disclosed platform and are not intended to limit the scope of the invention or imply any specific therapeutic outcome.

Although FIG. 20 illustrates one representative implementation architecture, the individual implementation layers may be combined, subdivided, reordered, supplemented, duplicated, automated, or modified according to the desired commercial application, manufacturing technology, regulatory environment, production capacity, or formulation requirements while remaining within the scope of the present invention.

Accordingly, FIG. 20 illustrates a comprehensive implementation architecture in which the raw material foundation layer (2410), biochemical preservation layer (2420), encapsulation and stabilization layer (2430), formulation integration layer (2440), manufacturing and quality layer (2450), delivery and dosage-form layer (2460), commercial implementation layer (2470), and consumer utilization layer (2480) collectively establish an integrated, scalable, and modular high-polyphenol olive oil nutraceutical delivery platform. The implementation architecture enables preservation of naturally occurring olive oil bioactive compounds while supporting diverse manufacturing methodologies, formulation strategies, dosage forms, quality-control systems, commercial product configurations, and consumer applications, thereby providing a versatile technological foundation for nutraceutical products incorporating high-polyphenol olive oil.

### INDUSTRIAL APPLICABILITY

The present invention is industrially applicable to the large-scale development, manufacture, quality control, preservation, packaging, storage, distribution, and commercial deployment of nutraceutical compositions incorporating high-polyphenol olive oil as a multifunctional biologically active delivery platform. The invention establishes a scalable manufacturing architecture that enables naturally occurring high-polyphenol olive oil to function not merely as a nutritional ingredient or lipid excipient, but as a structurally integrated delivery matrix capable of preserving, stabilizing, protecting, and delivering one or more co-formulated nutraceutical ingredients across numerous commercially viable dosage forms.

One significant industrial application resides in the nutraceutical manufacturing industry. Manufacturers of dietary supplements may utilize the disclosed quality characterization procedures to identify high-polyphenol olive oils possessing predetermined biochemical profiles suitable for standardized production. By incorporating analytical characterization prior to manufacturing, production facilities may reduce variability commonly associated with agricultural raw materials while producing nutraceutical formulations exhibiting substantially consistent biochemical characteristics between manufacturing batches.

The invention is further applicable to manufacturers producing oral solid dosage forms including tablets, chewable tablets, orally disintegrating tablets, fast-dissolving tablets, lozenges, capsules, multiparticulates, powders, granules, sachets, gummies, oral films, and related nutraceutical products. The encapsulation technologies described herein enable liquid high-polyphenol olive oil to be transformed into stable particulate structures compatible with conventional pharmaceutical and nutraceutical manufacturing equipment including tablet presses, capsule filling systems, powder blending systems, extrusion systems, granulation equipment, molding equipment, film-forming systems, and continuous manufacturing lines.

The disclosed encapsulation architecture is likewise applicable to industrial spray-drying facilities, freeze-drying facilities, coacervation systems, nanoemulsion production systems, liposomal manufacturing systems, cyclodextrin complex production facilities, lipid nanoparticle manufacturing systems, fluidized-bed coating equipment, and additional industrial encapsulation technologies. Because the invention is compatible with numerous encapsulation methodologies, manufacturers may select production technologies according to commercial capacity, production costs, desired release characteristics, and available manufacturing infrastructure.

Another important industrial application resides in production of antioxidant-sensitive nutraceutical formulations. Numerous nutraceutical ingredients—including botanical extracts, omega fatty acids, carotenoids, flavonoids, lipid-soluble vitamins, phospholipids, medicinal plant extracts, and additional oxidation-sensitive compounds—are susceptible to degradation during manufacturing and storage. The disclosed high-polyphenol olive oil delivery platform provides a naturally occurring antioxidant-rich lipid environment that may improve formulation stability while preserving the naturally occurring biochemical profile of the olive oil itself.

The invention is further applicable to manufacturers seeking to develop product families utilizing a common formulation platform. Because substantially the same encapsulated high-polyphenol olive oil delivery matrix may be incorporated into numerous dosage forms, manufacturers may efficiently develop multiple commercial products while minimizing reformulation of the underlying delivery technology. This modular platform architecture facilitates expansion of product portfolios while reducing formulation complexity, development time, and manufacturing costs.

The invention is additionally applicable to functional food manufacturing. The encapsulated high-polyphenol olive oil particles may be incorporated into nutritional beverages, wellness shots, protein supplements, nutritional bars, functional confectionery, powdered drink mixes, meal replacement products, fermented nutritional products, dairy-based nutritional systems, plant-based nutritional products, edible gels, frozen nutritional products, and numerous additional food products while preserving the structural integrity of the encapsulated olive oil particles during production and storage.

The invention further possesses industrial applicability within the pharmaceutical excipient and delivery technology industries. Although particularly suited for nutraceutical applications, the disclosed encapsulated lipid delivery matrix may function as a multifunctional formulation component capable of providing lipid-based delivery, controlled release, antioxidant preservation, stabilization, and manufacturing compatibility for additional orally administrable compositions where preservation of oxidation-sensitive ingredients is desirable.

The quality characterization architecture disclosed herein possesses independent industrial applicability for agricultural quality standardization. Olive oil producers, agricultural cooperatives, extraction facilities, quality-control laboratories, analytical service providers, and nutraceutical manufacturers may utilize the biochemical characterization procedures to classify olive oils according to objective analytical measurements rather than relying exclusively upon conventional commercial grading systems, geographic origin, or sensory evaluation.

The preservation methodologies described throughout the present specification likewise possess industrial applicability throughout commercial manufacturing and supply-chain management. Preservation-oriented harvesting procedures, extraction methodologies, storage systems, controlled-atmosphere packaging, oxygen-reduction systems, moisture-control technologies, environmental monitoring systems, and multilayer barrier packaging may collectively improve long-term preservation of naturally occurring phenolic compounds throughout manufacturing, warehousing, transportation, retail distribution, and consumer storage.

The invention further supports implementation within automated manufacturing facilities. The disclosed manufacturing workflows are compatible with programmable logic controllers, distributed control systems, industrial automation platforms, robotic material handling systems, continuous production lines, automated weighing systems, automated blending equipment, intelligent encapsulation systems, digital manufacturing execution systems, enterprise resource planning systems, laboratory information management systems, statistical process-control platforms, and industrial process monitoring technologies.

In preferred industrial implementations, manufacturing data generated throughout production may be digitally associated with individual olive oil batches, encapsulation processes, formulation records, packaging lots, analytical measurements, storage histories, transportation records, and distribution information. Such traceability supports regulatory compliance, production consistency, product recall management, inventory control, quality assurance, and commercial documentation throughout the manufacturing lifecycle.

The computer-implemented manufacturing management architecture described herein further enables integration with cloud computing systems, artificial intelligence-assisted quality optimization systems, predictive manufacturing algorithms, machine-learning quality-control systems, digital twin manufacturing environments, blockchain traceability platforms, environmental monitoring networks, and enterprise-scale manufacturing databases. Such digital integration facilitates continuous process optimization while preserving the biochemical integrity of the selected high-polyphenol olive oil.

The invention additionally supports contract manufacturing organizations (CMOs) and contract development and manufacturing organizations (CDMOs) engaged in nutraceutical production. Because the disclosed platform separates olive oil quality characterization, encapsulation, formulation, dosage-form manufacturing, packaging, and quality assurance into standardized modules, manufacturing operations may be distributed across multiple production facilities while maintaining consistent quality specifications.

Commercial implementation may encompass numerous consumer product categories including general dietary supplements, cardiovascular wellness products, healthy aging supplements, cognitive support formulations, immune-support products, sports nutrition formulations, gastrointestinal wellness products, metabolic wellness products, personalized nutrition systems, pediatric nutritional products, geriatric nutritional products, prenatal nutritional products, veterinary nutritional products, clinical nutrition products, medical foods, and additional nutritional compositions. These examples represent commercial implementation categories and are not intended to imply treatment, prevention, or cure of any disease.

The invention further exhibits industrial applicability across diverse manufacturing scales. Small-scale specialty nutraceutical manufacturers may implement batch-based production using conventional equipment, whereas large commercial manufacturers may implement continuous automated production utilizing industrial encapsulation technologies, robotic manufacturing systems, automated quality characterization, computerized process control, and high-throughput packaging systems. Consequently, the disclosed platform is readily adaptable from pilot-scale production through full-scale commercial manufacturing.

Because the invention preserves naturally occurring biochemical constituents while simultaneously improving manufacturability, dosage-form flexibility, formulation compatibility, storage stability, traceability, and commercial scalability, the disclosed high-polyphenol olive oil delivery platform is well suited for widespread industrial implementation throughout the nutraceutical, functional food, dietary supplement, formulation technology, encapsulation technology, food processing, analytical testing, packaging, quality-control, and commercial manufacturing industries. The invention therefore satisfies the requirement of industrial applicability by providing reproducible manufacturing methodologies capable of consistent commercial implementation using established industrial equipment and production technologies while preserving the naturally occurring biochemical characteristics of high-polyphenol olive oil.

### CONCLUSION

While the present invention has been described with reference to various exemplary embodiments, implementations, configurations, process flows, system architectures, encapsulation technologies, manufacturing methodologies, dosage forms, and delivery mechanisms, it will be understood by those skilled in the art that numerous modifications, substitutions, additions, omissions, variations, and alternative implementations may be made without departing from the spirit and scope of the invention as defined by the appended claims. The particular arrangements, materials, manufacturing processes, analytical techniques, encapsulation systems, release mechanisms, packaging configurations, computational implementations, and formulation strategies described herein are provided for purposes of illustration and explanation and should not be interpreted as limiting the invention.

Unless expressly stated otherwise, the use of terms such as “comprising,” “including,” “having,” “containing,” “configured to,” “adapted to,” and similar expressions is intended to be inclusive rather than exclusive, thereby permitting the presence of additional features, components, operations, materials, or process steps. Likewise, references to singular elements are intended to encompass plural implementations where appropriate, and references to plural elements likewise encompass singular implementations unless the context clearly indicates otherwise.

It should further be understood that individual features, elements, modules, subsystems, process steps, encapsulation layers, analytical procedures, dosage forms, preservation techniques, quality-control methodologies, packaging systems, computational functions, and manufacturing operations described in connection with one embodiment may be incorporated into, combined with, or substituted into other embodiments unless such combinations are technically incompatible. Accordingly, the various embodiments disclosed herein should be regarded as illustrative examples of broader inventive concepts rather than mutually exclusive implementations.

Furthermore, numerical values, concentration ranges, dimensions, processing conditions, environmental parameters, material selections, quality thresholds, release characteristics, and manufacturing specifications disclosed throughout the present specification are intended to represent exemplary embodiments and practical implementation examples. Such values may be modified, expanded, optimized, or otherwise varied according to particular commercial applications, manufacturing capabilities, regulatory requirements, raw material characteristics, or desired product performance while remaining within the scope of the present invention.

The present invention provides a comprehensive and integrated high-polyphenol olive oil nutraceutical delivery platform in which naturally occurring high-polyphenol olive oil functions as a multifunctional biologically active delivery matrix rather than merely as a conventional edible oil or inert lipid carrier. Through coordinated implementation of biochemical characterization, quality-based olive oil selection, advanced encapsulation technologies, preservation-oriented manufacturing processes, configurable formulation architectures, controlled-release delivery systems, protective storage methodologies, computer-assisted manufacturing management, and broad dosage-form adaptability, the invention enables preservation of the naturally occurring biochemical profile of high-polyphenol olive oil while supporting stable incorporation and coordinated delivery of one or more co-formulated nutraceutical ingredients. Collectively, these technological features establish a versatile, scalable, and commercially practical platform capable of supporting the development and manufacture of diverse nutraceutical products while maintaining the structural, biochemical, and functional characteristics associated with premium high-polyphenol olive oil.

1. A nutraceutical delivery platform, comprising:
a high-polyphenol olive oil selected according to one or more predetermined biochemical characteristics including naturally occurring phenolic compounds;
a biochemical characterization system configured to determine one or more biochemical parameters of the high-polyphenol olive oil;
an encapsulation system configured to encapsulate the high-polyphenol olive oil within one or more protective encapsulating structures;
at least one nutraceutical ingredient combined with the encapsulated high-polyphenol olive oil;
a formulation architecture configured to incorporate the encapsulated high-polyphenol olive oil into an orally administrable dosage form; and
a controlled-release architecture configured to preserve the naturally occurring biochemical profile of the high-polyphenol olive oil until a predetermined stage of physiological administration,
wherein the encapsulated high-polyphenol olive oil functions as a biologically active delivery matrix that supports preservation and coordinated delivery of the at least one nutraceutical ingredient.

2. The nutraceutical delivery platform of claim 1, wherein the biochemical characterization system determines one or more of:
total polyphenol concentration;
hydroxytyrosol concentration;
tyrosol concentration;
oleuropein derivative concentration;
oleocanthal concentration;
oleacein concentration;
ligstroside derivatives;
tocopherol concentration;
phytosterol concentration;
flavonoid concentration;
phenolic acid concentration;
triterpene concentration;
antioxidant capacity;
oxidative stability;
peroxide value;
free fatty acid concentration; and
moisture content.

3. The nutraceutical delivery platform of claim 1, wherein the high-polyphenol olive oil possesses a total naturally occurring phenolic concentration greater than approximately 300 mg/kg.

4. The nutraceutical delivery platform of claim 1, wherein the encapsulation system comprises one or more of:
spray drying;
freeze drying;
liposomal encapsulation;
nanoemulsion formation;
microemulsion formation;
cyclodextrin inclusion complex formation;
coacervation;
lipid nanoparticle formation;
solid lipid particle formation;
hydrocolloid encapsulation;
polysaccharide encapsulation;
protein matrix encapsulation; or
multilayer encapsulation.

5. The nutraceutical delivery platform of claim 1, wherein the encapsulation system comprises:
an olive oil core;
an inner stabilization layer;
an intermediate barrier layer;
an outer protective layer; and
an optional release-regulating layer.

6. The nutraceutical delivery platform of claim 1, wherein the encapsulated high-polyphenol olive oil is substantially uniformly dispersed throughout a structural formulation matrix.

7. The nutraceutical delivery platform of claim 1, wherein the nutraceutical ingredient comprises one or more of:
vitamins, minerals, botanical extracts, probiotics, prebiotics, amino acids, peptides, phospholipids, enzymes, omega fatty acids, carotenoids, flavonoids, medicinal mushroom extracts, proteins, marine-derived compounds, fermentation products, functional fibers, trace minerals, or adaptogens.

8. The nutraceutical delivery platform of claim 1, wherein the controlled-release architecture provides one or more of:
immediate release;
delayed release;
sustained release;
sequential release;
staged release;
pulsatile release;
pH-responsive release;
enzyme-responsive release;
moisture-responsive release; or
site-specific release.

9. The nutraceutical delivery platform of claim 1, wherein release of the encapsulated olive oil establishes a localized lipid microenvironment surrounding the nutraceutical ingredient.

10. The nutraceutical delivery platform of claim 9, wherein the localized lipid microenvironment promotes dispersion of lipophilic, amphiphilic, and hydrophilic nutraceutical ingredients.

11. A method of manufacturing a nutraceutical composition, comprising:
selecting a high-polyphenol olive oil according to predetermined biochemical characteristics;
performing biochemical characterization of the selected olive oil;
determining whether the olive oil satisfies predetermined quality specifications;
encapsulating the selected olive oil to form encapsulated olive oil particles;
combining the encapsulated olive oil particles with one or more nutraceutical ingredients;
forming an orally administrable dosage form;
packaging the dosage form within a preservation-oriented packaging system; and
preserving the naturally occurring biochemical profile of the high-polyphenol olive oil throughout manufacturing and storage.

12. The method of claim 11, wherein biochemical characterization comprises chromatographic analysis, spectroscopic analysis, antioxidant analysis, compositional analysis, or combinations thereof.

13. The method of claim 11, wherein encapsulation comprises formation of multilayer encapsulated particles including an olive oil core and one or more protective layers.

14. The method of claim 11, wherein the dosage form comprises one or more of:
a lozenge;
chewable tablet;
orally disintegrating tablet;
fast-dissolving tablet;
gummy;
capsule;
oral film;
powder;
granule;
sachet;
beverage; or
functional confectionery product.

15. The method of claim 11, further comprising monitoring one or more manufacturing parameters selected from:
temperature, humidity, oxygen concentration, moisture, encapsulation efficiency, particle size, formulation homogeneity, dissolution profile, antioxidant activity, or phenolic retention.

16. A computer-implemented manufacturing management system for producing nutraceutical compositions comprising high-polyphenol olive oil, the system comprising:
a data acquisition subsystem;
a biochemical characterization database;
a manufacturing process controller;
a production monitoring subsystem;
a quality assurance engine;
a batch traceability subsystem;
a packaging and inventory subsystem; and
a reporting subsystem,
wherein the system digitally associates biochemical characterization data of a selected high-polyphenol olive oil with one or more manufacturing operations associated with production of the nutraceutical composition.

17. The system of claim 16, wherein the manufacturing process controller automatically adjusts one or more encapsulation or manufacturing parameters based upon the biochemical characterization data.

18. The system of claim 16, wherein the batch traceability subsystem maintains digital records associating a finished nutraceutical product with:
an olive oil source;
biochemical profile;
encapsulation process;
formulation batch;
manufacturing history;
packaging information; and
distribution information.

19. A configurable nutraceutical formulation platform, comprising:
a standardized encapsulated high-polyphenol olive oil delivery matrix;
a formulation configuration subsystem configured to selectively combine the delivery matrix with different nutraceutical ingredient combinations;
a dosage-form adaptation subsystem configured to produce a plurality of different oral dosage forms from the standardized delivery matrix,
wherein substantially the same encapsulated high-polyphenol olive oil delivery matrix is incorporated into multiple commercially distinct nutraceutical products.

20. A nutraceutical composition, comprising:
a plurality of encapsulated particles each containing a high-polyphenol olive oil selected according to a predetermined biochemical profile;
one or more nutraceutical ingredients dispersed with the encapsulated particles;
and a structural formulation matrix supporting the encapsulated particles,
wherein the encapsulated particles preserve naturally occurring phenolic constituents of the high-polyphenol olive oil and, upon release, establish a lipid-assisted delivery environment configured to facilitate coordinated delivery of the one or more nutraceutical ingredients.
